(19) United States
(12) Patent Application Publication
(10) Pub. No.: US 2025/0268840 A1
(43) Pub. Date: Aug. 28, 2025

(54) USE OF NANO-CARRIERS FOR DELIVERY OF ACTIVE AGENTS

(71) Applicant: LANDA LABS (2012) LTD., Rehovot (IL)

(72) Inventors: Sagi ABRAMOVICH, Ra'anana (IL); Gal AVIDOR, Rehovot (IL); Benzion LANDA, Nes Ziona (IL)

(73) Assignee: LANDA LABS (2012) LTD., Rehovot (IL)

(21) Appl. No.: 19/207,406

(22) Filed: May 14, 2025

Related U.S. Application Data
(63) Continuation-in-part of application No. PCT/IB2023/061681, filed on Nov. 20, 2023.

(30) Foreign Application Priority Data
Nov. 22, 2022 (GB) 2217477.5
Jul. 13, 2023 (WO) PCT/IB2023/057187

(51) Int. Cl. A61K 9/51; A61K 47/10; A61K 47/44

(52) U.S. Cl. CPC A61K 9/5146; A61K 47/10; A61K 47/44

(57) ABSTRACT

There are disclosed methods of treating a subject or an object in need thereof, the method comprising administering compositions comprising nano-elements containing: a) at least one water-insoluble thermoplastic compound (WITC), capable of forming a core; and b) at least one active agent which can be disposed in said core or in shells surrounding the core. The nano-elements, having an average diameter in the sub-micron range, are constituted of materials having a low vapor pressure and are dispersible in a polar carrier. Methods for preparing these nano-elements, and administering them, so as to treat conditions corresponding to the active agents contained therein, are also provided.

### CROSS-REFERENCE TO RELATED APPLICATIONS

This application is a Continuation-in-Part of International Application No. PCT/IB2023/061681, filed on Nov. 20, 2023, which claims Paris Convention priority from Great-Britain Patent Application No. 2217477.5 filed on Nov. 22, 2022, and from International Patent Application No. PCT/IB2023/057187 filed on Jul. 13, 2023. The contents of the afore-mentioned applications are hereby incorporated by reference in their entirety for all purposes as if fully set forth herein.

### FIELD

The present disclosure relates to compositions comprising nano-particles suitable for delivery of active agents and their use. Methods of preparing these compositions and using them in treatment are also provided.

### BACKGROUND

Medications are taken to diagnose, prevent or treat illnesses. They exist in numerous pharmaceutical forms and can be administered to an animal or human subject by various routes. Typically, the active ingredients of such compositions, enabling their use for the diagnosis or treatment of a disease, are administered as an admixture with suitable pharmaceutically acceptable excipients which are selected according to the intended form and route of administration. Such medicaments can be introduced into the body by several routes (e.g., local or systemic including enteral and parenteral routes) each having its own advantages or disadvantages for a specific purpose and/or disease.

Nanotechnology has been proven advantageous in drug development, in particular for drug formulation and delivery. The nano-particles produced by such technology, having a particle size generally between 10 nanometer (nm) and 1,000 nm, allow, among other things, site-specific and target-oriented delivery of medicines due to the ability of the nano-sized particles to, e.g., cross the blood brain barrier (BBB), enter the pulmonary system, or be absorbed through the tight junctions of endothelial cells of blood vessels, if small enough (e.g., having a particle size of up to 200 nm or even up to 100 nm). It has been argued that if the medicine is itself a small enough molecule having a molecular weight not exceeding about 1,000 g/mol, and being preferably smaller than 500 g/mol as known from Lipinski's “Rule of 5”, it might even be capable of intracellular delivery. These nano-sized particles can be in the form of nano-spheres, nano-capsules, nano-crystals, nano-emulsions, nano-fibers, nano-tubes, polymeric micelles, polymersomes, dendrimers, liposomes, etc.

Various compounds, such as polymers (biodegradable or not), are used in nano-medicine as nano-carriers of therapeutic agents, allowing the transfer of such agents into the target sites, while protecting the drug from premature degradation and/or reducing premature interaction of the drug with the biological environment. Such nano-carriers can also allow controlled or delayed release of medications at a target site. The polymers, whether homo-polymers, mixed polymers or copolymers, can either serve to form the envelop of a hollow vesicle, the drug being within the core of such polymersomes and/or intercalated in the surrounding membrane, or serve to embed the drug within the polymer matrix constituting the entire nano-carrier.

Various conventional methods for preparing these nano-carriers (e.g., polymeric), are described in the literature but most of them rely on the use of solvents which typically remain residually trapped within such traditionally prepared particles. When the solvent being used is a volatile organic compound (VOC), numerous drawbacks are known, since despite their relative volatility, such solvents cannot be fully eliminated beyond a residual level which can be non-negligible. Firstly, nano-carriers serving in the pharmaceutical industry must comply with the guidelines issued by The International Council for Harmonisation of Technical Requirements for Pharmaceuticals for Human Use (ICH), so that the amount of such residual VOCs should be limited accordingly in view of their toxicity. Secondly, as a VOC may gradually evaporate over time until it reaches its residual level, the nano-particles' structure or properties may be altered during this period, consequently affecting the efficacy of the drug carried thereby, such as the drug release profile. While cumbersome methods have been developed in an attempt to address the residual entrapment of undesired solvents, the approaches reported so far fail to provide nano-particles, or even micro-particles having a narrow size distribution, and/or may lack commercial feasibility in view of their complexity, their failure to form stable dispersions, or their low encapsulation efficiency, when active ingredients are additionally desired.

Despite the intensive research in the field of drug delivery via nano-carriers aimed to increase the stability of the carried drug, its loading, or its selective targeting and controlled release at a site of relevance, there remains a need for such nano-carriers. Advantageously, such carriers may display a low concentration of volatile organic solvents and permit the delivery of water-insoluble drug molecules having a relatively high average molecular weight.

### SUMMARY

Aspects of the invention relate to uses of a composition for the preparation of a medication adapted for administration to living subjects or animate objects, the composition comprising a water-insoluble thermoplastic compound (WITC), such as a water-insoluble thermoplastic polymer (WITP), which can be dispersed as nano-particles or nano-droplets (collectively referred to as “nano-elements”) in a polar liquid carrier. These nano-particles or nano-droplets may serve for the delivery of at least one active agent, and hence, they may also be referred to as nano-carriers.

The nano-elements may be in the form of core nano-elements, wherein the cores comprising the WITC(s) and the active-agent(s) are not further enveloped by an external shell. Alternatively, the nano-elements may be in the form of core-shell, or core-multi-shells nano-elements, wherein the active agent(s) may be within the core comprising the WITC(s) and/or forming the shell(s) directly or indirectly surrounding the core, as will be explained below. Notably, the nano-particles or nano-droplets made therewith contain less than 2 wt. % of a volatile organic compound. Moreover, in view of the methods by which they can be prepared, the core of the nano-elements is believed to form a continuous phase, resulting in a non-porous core. The WITCs can in some cases be biodegradable and/or can be modified to enhance any of their desired properties (e.g., with respect to the release of active agents).

As used herein, the term “nano-elements”, referring to the structures containing inter alia the WITC (plasticized or not) and the active agent(s), regardless of where such agents are disposed with respect to the core, refers to particles typically globular which at room temperature can be relatively solid nano-particles or relatively liquid nano-droplets having an average diameter of 1,000 nm or less, 750 nm or less, 500 nm or less, or 250 nm or less, and in particular of 200 nm or less, 190 nm or less, 175 nm or less, 150 nm or less, 125 nm or less, 100 nm or less, 90 nm or less, 80 nm or less, 75 nm or less, or 50 nm or less, such structures being dispersible (e.g., as a result of nano-sizing) in a homogeneous medium, and capable of forming therein a nano-suspension. Such nano-elements have typically an average diameter of 2 nm or more, 5 nm or more, 10 nm or more, 15 nm or more, or 20 nm or more. In some embodiments, the average diameter of the nano-elements of the compositions that can be used for the preparation of medications according to the present teachings is between 2 nm and 1,000 nm, between 2 nm and 750 nm, between 2 nm and 500 nm, or between 2 nm and 250 nm. In other embodiments, the average diameter of the nano-elements is between 2 nm and 200 nm, between 2 nm and 190 nm, between 2 nm and 175 nm, between 5 nm and 150 nm, between 5 nm and 125 nm, between 10 nm and 100 nm, between 10 nm and 90 nm, between 10 nm and 80 nm, between 15 nm and 75 nm, between 15 nm and 70 nm, or between 20 nm and 50 nm. The average diameter of the nano-elements can be determined by any suitable method and may refer to the hydrodynamic diameter of the elements as measured by Dynamic Light Scattering (DLS) and established for 50% of the nano-elements by number (D_{N}50) or by volume (D_{V}50). In particular embodiments, the average diameters of the nano-elements are determined by number.

While the nano-elements are inter alia characterized by their dispersibility in a polar liquid carrier, the nano-elements being for instance hydrophobic and the liquid aqueous, a composition to be administered to a subject or an animate object in need thereof, is not necessarily in liquid form. The nano-elements can be isolated from a polar liquid carrier in which they could be dispersed, so as to serve for the preparation of the desired medications in dry forms. Therefore, while the nano-elements included in the present medications are often referred to as being “dispersed”, when the composition is liquid, this term encompasses nano-elements that would be “dispersible”, if in a suitable liquid environment.

Dispersible nano-elements are, when dispersed in a polar liquid carrier, generally characterized by being discrete individual nano-elements, separated from one another, with minimal aggregation or agglomeration, thus stably maintaining their particle size distribution (PSD) as herein-described over an extended period of time (e.g., the dispersion being stable with insignificant fluctuation in PSD for at least 1 day). The dispersibility can be confirmed by microscopic means, analyzing the particle size distribution of the nano-elements by standard methods (such as DLS).

These compositions may be used for the benefit of living animals (e.g., mammalian or non-mammalian, human or non-human) or animate objects (e.g., seasonal or perennial plants and parts or products thereof). Thus, the medications prepared therewith can be referred to by various denominations depending on the targeted population, the intended use of the active agents contained in the nano-elements (in the core and/or shell(s) thereof), and/or the routes of administration. Regarding the latter, the present invention is specifically concerned with administration by routes other than the visible skin of living animals or other than the external/exposed surface of objects. For simplicity, all routes of administration other than the afore-said exclusions can be referred to as “non-exposed-surface administration”, or “internal administration”, even when relating to objects, and in particular can be referred to as “non-dermal administration” when the subject is a living animal, the surface excluded from the present routes of administration being its exposed skin. The routes by which the present medications can be administered can also be termed for conciseness NESA (or NDA), or NES (or ND) administration, and like grammatical variants. The acronyms NES or ND can be similarly employed with additional terms when referring to the medications and/or the uses of the present compositions as can be administered by NES or ND routes.

These compositions or medications made therewith when intended for administration to living subjects are generally called pharmaceutical products, this terminology including veterinary products when the living subjects are non-human animals. Pharmaceutical products can be used for delivery of active agents by various routes, including local routes, for an effect substantially limited to the site of administration (e.g., oral, ocular, auricular, rectal, nasal, vaginal, and through like non-exposed cavities of the body of the subject), and systemic routes, for a system-wide effect (i.e., enteral or parenteral, such as injection, sublingual, inhalation, nebulization, intrathecal, epidural, etc.). As illustrated by the exemplary routes of administration to a living subject, some of the organs that may be treated by the present medications, or that may serve as point of entry to the active agents carried therewith, have exposed portions including skin, such as eyelids, yet the envisioned delivery is considered non-dermal (via ocular surfaces, via mucous membranes, via inner portions of the organ, etc.).

When used to prepare agrochemical products, the compositions may deliver dedicated active agents by injection into a plant or a relevant part thereof (e.g., the trunk or branches of a tree). Drenching of the soil surrounding the plant with the agrochemical product may also enable systemic delivery of the active agents by absorption through the roots.

As used herein, the term “active agent(s)” refers to any substance capable of diagnosing, preventing, ameliorating, attenuating, delaying or arresting a progression and/or cure the condition being treated by the NES (e.g., ND) administration of the medication, or any like effects commonly associated with active agents as known to skilled persons. For illustration, such agents include substances commonly known as drugs, as well as food supplements (e.g., electrolytes, vitamins, minerals, metals, and any like nutrients) and such alternative substances conventionally known to improve the health or wellness of individuals. Thus, by analogy, a “medication” refers to a composition including such active agents which may provide a similar diagnostic, prophylactic or therapeutic effect (regardless of degree) with respect to the targeted condition, the medication being typically formulated according to the NES route it is to be administered by. Any and all such effects that can be achieved by the active agents or the medications containing the same are considered as a “treatment” of the subject or object to whom/which the medication is to be administered, this term encompassing the detection of ailments.

The present medications can be NES (e.g., ND) administered to provide on their own for any such sought treatment or may serve to increase the efficacy of co-administered treatments directed to a same condition, regardless of the mode of administration of the co-treatment and/or of its composition. The extent of treatment or co-treatment, with meanings as afore-exemplified, by a medication prepared and administered according to the present teachings, hence their efficacy, may be assessed, by a diminution of the symptoms relevant to the condition being treated or by an increase in an activity relevant to a property intended to be desirably enhanced.

In a first aspect of the disclosure, there are provided uses of a composition for the preparation of a medication for the treatment of a living subject or of an object by administration of the medication through non-exposed surfaces (e.g., by ND routes) of said subject or object, the composition comprising nano-elements dispersible in a polar carrier, the nano-elements containing:
a) at least one water-insoluble thermoplastic compound (WITC) forming a core of each nano-element; and
b) at least one active agent being miscible in the WITC(s) and insoluble in the polar carrier, so as to be disposed within the core;
   wherein each constituent of the nano-elements has a vapor pressure of 40 Pascal (Pa) or less, as measured at a temperature of about 20° C.; and wherein the nano-elements have an average diameter (e.g., D_{N}50) of 1,000 nm or less.

Active agents being miscible in the WITC(s) (hence, referred to as a “WITC-miscible” active agent) and insoluble in the polar carrier (hence, referred to as a “polar-carrier-insoluble active agent” or a “carrier-insoluble active agent”) can be referred to as being “WITC-miscible/polar-carrier-insoluble”. As used herein, a component is deemed to be “insoluble” within the liquid polar carrier (e.g., water) when its solubility within the carrier is less than 5 wt. %, and more typically, less than 4 wt. %, less than 3 wt. %, less than 2 wt. %, less than 1 wt. %, or less than 0.5wt. %, by weight of the polar carrier, at a temperature of 20° C. Furthermore, such components are considered “miscible” within the WITC if capable of forming a homogeneous mixture with WITCs without causing phase separation or any other like alteration of the blend due to form the continuous core, such WITC-miscible materials generally have a solubility within the WITC of 5wt. % or more (and more typically, 6 wt. % or more, 7 wt. % or more, 8 wt. % or more, 9 wt. % or more, or 10 wt. % or more) by weight of the WITC they are mixed with.

In some embodiments, the nano-elements (isolated from a liquid medium) have a dynamic viscosity of 10⁷ millipascal-second (mPa·s) or less, 10⁶ mPa·s or less, 10⁵ mPas or less, 10⁴ mPa·s or less, or 10³ mPa·s or less, as measured at at least one temperature between 20° C. and 80° C., and at a shear rate of 10 sec⁻¹. In some embodiments, the nano-elements have a dynamic viscosity of at least 1 mPa·s, at least 10 mPa·s, or at least 100 mPa·s, under the same measuring conditions. Such viscosity of the nano-elements can result from the native viscosity of the, or each, WITC, or of their blend when more than one, or can result from the presence of the active agent(s) (or any other compound to be later discussed) in the WITC(s). In other cases, a non-volatile liquid can be present in the core, for the purpose of plasticizing the WITC(s) so as to reduce the viscosity of the nano-elements of the present medications to be within the afore-said ranges.

The viscosity of the bulk WITC(s) serving to prepare the nano-elements can be referred to as a first (or a native) viscosity of the WITC, or of a blend thereof, whereas the viscosity of the nano-elements containing at least the WITC(s) and the active agent(s) can be referred to as a second viscosity. More generally, a property characterizing the bulk WITC(s) contained in the core can be called a “first” property, while an analogous property as measured on the nano-elements can be referred to as a “second” property, which can have same or different value as compared to the native WITC.

In some embodiments, the at least one WITC (and/or the nano-elements comprising it, optionally as a WITC plasticized by a non-volatile liquid) is characterized by at least one, at least two, or at least three of the following structural properties:
i. the WITC and/or the nano-elements is/are insoluble in the polar carrier;
ii. the WITC and/or the nano-elements is/are biodegradable and/or biocompatible;
iii. the WITC and/or the nano-elements has/have at least one of a melting temperature (Tm), a softening temperature (Ts), or a glass transition temperature (Tg) of at most 300° C., at most 250° C., at most 200° C., at most 180° C., at most 150° C., or at most 120° C., said temperatures being either a first (i.e., native) Tm, Ts or Tg of the WITC, or a second Tm, Ts or Tg of the nano-elements, or both;
iv. the WITC and/or the nano-elements has/have each respectively a first and/or second Tm of at least 0° C., at least 10° C., at least 20° C., at least 30° C., at least 40° C., at least 50° C., or at least 60° C.;
v. the WITC and/or the nano-elements has/have each respectively at least one of a first and/or second Tm between 0° C. and 300° C., between 10° C. and 300° C., between 20° C. and 300° C., between 20° C. and 250° C., between 20° C. and 200° C., between 30° C. and 180° C., between 40° C. and 150° C., or between 50° C. and 120° C.;
vi. the WITC and/or the nano-elements has/have a first and/or second Tg or Ts of-75° C. or more,-50° C. or more,-25° C. or more, 0° C. or more, 10° C. or more, 20° C. or more, 25° C. or more, 30° C. or more, 40° C. or more, 50° C. or more, or 60° C. or more;
vii. the WITC and/or the nano-elements has/have at least one of a first and/or second Ts or Tg between −75° C. and 300° C., between −50° C. and 250° C., between −25° C. and 200° C., between 0° C. and 180° C., between 20° C. and 300° C., between 20° C. and 250° C., between 20° C. and 200° C., between 20° C. and 180° C., between 30° C. and 180° C., between 40° C. and 180° C., between 30° C. and 150° C., between 50° C. and 150° C., or between 50° C. and 120° C.;
viii. the WITC and/or the nano-elements has/have each respectively a first and/or a second viscosity of 10⁷ mPa·s or less, 5×10⁶ mPa·s or less, 10⁶ mPa·s or less, 5×10⁵ mPa·s or less, 10⁵ mPa·s or less, 5×10⁴ mPa·s or less, 10⁴ mPa·s or less, 5×10³ mPa·s or less, or 10³ mPa·s or less, as measured at at least one temperature between 20° C. and 80° C., and at a shear rate of 10 sec⁻¹;
ix. the WITC has a molecular weight of 0.5 kDa or more, 0.7 kDa or more, 0.8 kDa or more, 0.9 kDa or more, 1 kDa or more, 1.5 kDa or more, 2 kDa or more, 2.5 kDa or more, 3 kDa or more, 3.5 kDa or more, 4 kDa or more, 4.5 kDa or more, 5 kDa or more, 5.5 kDa or more, 6 kDa or more, 6.5 kDa or more, or 7 kDa or more;
x. the WITC has a molecular weight of 500 kDa or less, 300 kDa or less, 200 kDa or less, 100 kDa or less, 80 kDa or less, 50 kDa or less, 25 kDa or less, or 15 kDa or less; and
xi. the WITC has a molecular weight between 0.5 kDa and 500 kDa, 0.6 kDa and 500 kDa, between 0.7 kDa and 300 kDa, between 0.8 kDa and 200 kDa, between 1 kDa and 100 kDa, between 2 kDa and 80 kDa, between 1.5 kDa and 500 kDa, between 2.5 kDa and 300 kDa, between 3 kDa and 200 kDa, between 3.5 kDa and 500 kDa, between 4 kDa and 500 kDa, between 5 kDa and 300 kDa, between 5.5 kDa and 300 kDa, between 6 kDa and 200 kDa, between 6.5 kDa and 200 kDa, or between 7 kDa and 200 kDa.

In some embodiments, the at least one structural property fulfilled by at least one of the WITC and the nano-elements (optionally including a plasticized WITC) is: property i) as above listed, property ii) as above listed, property iii) as above listed, property iv) as above listed, property v) as above listed, property vi) as above listed, property vii) as above listed, property viii) as above listed; property ix) as above listed; property x) as above listed; or property xi) as above listed.

In some embodiments, the at least two structural properties fulfilled by at least one of the WITC and the nano-elements comprising the (optionally plasticized) WITC are: properties i) and ii); properties i) and v), properties i) and vii); properties i) and viii), properties i) and xi); properties ii) and v); properties ii) and vii); properties ii) and viii); properties ii) and xi); properties v) and xi), properties vii) and xi); or properties viii) and xi) of the above-listed properties.

In some embodiments, the at least three structural properties fulfilled by at least one of the WITC and the nano-elements comprising the (optionally plasticized) WITC are: properties i), ii) and iii); properties i), ii) and v); properties i), ii) and vii); properties i), ii) and viii); properties i), ii) and xi); properties i), v) and vii); properties i), v) and viii); properties i), vii) and viii); properties i), v) and xi); properties i), vii) and xi); or properties i); or viii) and xi), of the above-listed properties.

In particular embodiments, the water-insoluble thermoplastic compound (WITC) is a water-insoluble thermoplastic polymer (WITP), formed of repeating structural units, such monomers being either same (forming homopolymers) or different (forming random or block copolymers). While non polymeric compounds typically have molecular weights of up to 2 kDa, generally not exceeding 1 kDa, WITPs can be larger molecules of at least a few kDas.

In view of their intended use and/or method of preparation, WITCs suitable for the medications of the present invention are advantageously, but not necessarily, relatively solid at room temperature (circa 25° C.) and up to body temperatures (e.g., circa 37° C. for human subjects). Such preferences are extended to the nano-elements containing them, which further takes into account the presence of any material affecting the thermal behavior of the product, such as the active agent and/or the non-volatile liquid and their respective relative amounts. As can be appreciated by persons skilled in the art, as the WITCs can be thermoplastic polymers, a “relative solidity” of such materials, or such materials being “relatively solid”, at any particular temperature is referring to the fact that they are not necessarily solid but display a viscoelastic behavior. Without wishing to be bound by any particular theory, such feature of the WITCs should ensure, to the extent necessary, that the nano-elements made therefrom are relatively non-sticky, facilitating their even distribution in a composition or medication according to the present teachings.

The compositions can be incorporated in the medications of the present invention in the form of a nano-suspension. Depending on the Tm or Ts of the WITCs forming the core of the nano-elements, the composition can be at room temperature in the form of a nano-dispersion (i.e., if the Tm or Ts is above 25° C., e.g., between 25° C. and 80° C.), the nano-elements being relatively solid nano-particles, or alternatively be in the form of a nano-emulsion (i.e., if the Tm or Ts is below 25° C.), the nano-elements being relatively liquid nano-droplets. Alternatively, the nano-elements, while dispersible in a polar liquid and capable of forming therewith a nano-dispersion or a nano-emulsion, are used in the preparation of a medication in a form isolated from such media. For illustration, the present compositions could be added in liquid form to the solid excipients of the dry dosage form and dried and compacted therewith, or freeze dried and then added to the excipients.

The WITC (e.g., WITP) can be a natural compound, isolated fractions thereof and/or chemically processed versions of the same, even if artificially synthesized, or synthetic equivalents or derivatives of said materials, having no natural occurrence but being functionally comparable or preferable to the native materials as can be found in nature. Some compounds suitable for the present teachings may, under particular circumstances, assemble to form larger molecules considered then as polymers. Such compounds shall be referred to as polymerizable before such connections are made.

In some embodiments, the polymerizable WITC is a natural compound selected from: resins, gums, gum-resins, waxes and combinations thereof. In a particular embodiment, the polymerizable WITC is shellac or gum rosin.

In some embodiments, the WITC is non-polymerizable, such as a quinone, in particular ubidecarenone, also called 1,4-benzoquinone or coenzyme Q10 (CoQ10).

In other embodiments, the WITC is a WITP, which may also be of synthetic or natural origin. In some embodiments, the thermoplastic polymer is a biodegradable polymer, selected from a group of polymer families comprising: aliphatic polyesters, polyhydroxy-alkanoates, poly(alkene dicarboxylates), polycarbonates, aliphatic-aromatic co-polyesters, isomers thereof, copolymers thereof and combinations thereof. While some of the afore-said polymers have natural counterparts, these exceptions are typically commercially available almost exclusively in artificially prepared form, so that the entire group is often considered as representative of synthetic polymers.

Alternatively, the thermoplastic polymer can be a non-biodegradable synthetic polymer such as a polyamide (PA), a polyethylene (PE), a poly(ethylene-co-acrylic acid) (PEAA), a poly(ethylene-co-methacrylic acid) (PEMAA), a poly(ethylene-co-n-butyl acrylate) (PEBA), a poly(ethylene-co-vinyl acetate) (PEVA), a polymethylmethacrylate (PMMA), a polypropylene (PP), a polysiloxane, a polystyrene (PS), a polytetrafluoroethylene (PTFE), polyurethane (PU), or a polyvinyl chloride (PVC).

In other embodiments, the WITP is a natural biodegradable polymer selected from polysaccharides, lignin and combinations thereof.

In particular embodiments, the WITC is a biodegradable material such as CoQ10, shellac, gum rosin or a WITP being an aliphatic polyester. In a further particular embodiment, the aliphatic polyester is selected from polycaprolactone, polylactic acid, poly(lactic-co-glycolic acid), poly(butylene succinate-co-adipate), isomers thereof, copolymers thereof and combinations thereof.

In some embodiments, the non-volatile liquid that may be added to the WITC to lower at least one of its first (native) viscosity, Tm, Tg and Ts is selected from a group comprising: monofunctional and polyfunctional aliphatic esters, aromatic esters, fatty esters, cyclic organic esters, terpenes, aromatic alcohols, aromatic ethers, aldehydes and combinations thereof. In some embodiments, the non-volatile liquid is an aliphatic or aromatic fatty ester. In particular embodiments, the non-volatile liquid is selected from: dibutyl adipate, dibutyl sebacate, benzyl benzoate, triethyl O-acetylcitrate, C₁₂-C₁₅ alkyl benzoate and dicaprylyl carbonate.

In other embodiments, when the polymer is a non-biodegradable synthetic polymer, the non-volatile liquid may additionally be selected from a group comprising mineral oils, natural oils, vegetal oils, essential oils, synthetic oils and combinations thereof, provided that they satisfy the present requirements. Non-limiting examples of synthetic oils include synthetic isoparaffins (e.g., Isopar™ M and Isopar™ V), C₁₂-C₁₅ alkyl ethylhexanoate, C₁₂-C₁₅ alkyl benzoate, or isononlyl isononanoate, to name but a few. Non-limiting examples of suitable vegetal oils include castor oil, corn oil, pomegranate seeds oil, or avocado oil, to name but a few. Non-limiting examples of essential oils include clove leaf oil, lavender oil, or oregano oil, to name but a few.

In some embodiments, the polar carrier in which the nano-elements comprising the WITC and the carrier-insoluble active agent are dispersible (e.g., if the composition is in dry form) or dispersed (e.g., if the composition is in liquid form) comprise water, glycols (e.g., propylene glycol, 1,3-butanediol, 1,4-butanediol, 2-ethyl-1,3-hexanediol and 2-methyl-2-propyl-1,3-propanediol), formamide, acetonitrile, glycerol, precursors and derivatives thereof, collectively termed herein “glycerols” (e.g., acrolein, dihydroxyacetone, glyceric acid, tartronic acid, epichlorohydrin, glycerol tertiary butyl ether, polyglycerol, glycerol ester and glycerol carbonate) and combinations thereof. In a particular embodiment, the polar carrier comprises water, consists of water or is water.

In some embodiments, the composition further comprises at least one surfactant, selected from an emulsifier and a hydrotrope. The surfactant(s) may be present in the nano-elements containing the WITC (e.g., if being polar-carrier-insoluble surfactant(s)), in the liquid phase containing the polar carrier (e.g., if being polar-carrier-soluble surfactant(s)), or in both (e.g., if being intermediate emulsifiers).

In some embodiments, the compositions used for the preparation of the present medications comprise more than one active agent in addition to the one embedded in the WITC nano-elements, the additional active agents being either in same or different phase. For illustration, a first additional active agent, being WITC-miscible/polar-carrier-insoluble, can be contained within the nano-elements and a second additional active agent, being carrier-soluble, can be contained within the polar carrier phase.

An active agent is deemed to be soluble in a liquid carrier, being for instance a “polar-carrier-soluble active agent” (or a “carrier-soluble active agent”), if having a solubility within the carrier it is immersed in of 5 wt. % or more (and more typically, 6 wt. % or more, 7 wt. % or more, 8 wt. % or more, 9 wt. % or more, or 10 wt. % or more) by weight of the polar carrier, at a temperature of 20° C. As appreciated by a skilled person some materials having a sought activity can be polar-carrier-insoluble in one chemical form, and polar-carrier-soluble in another, salts of a material typically increasing its solubility.

Advantageously, the compositions serving for the preparation of the present medications can have a) a relatively high concentration (e.g., 1 wt. % or more) of a WITC (e.g., a WITP) and/or of a WITC-miscible/carrier-insoluble active agent embedded therein, and/or b) the WITC(s) and/or the active agent(s) embedded therein can have a relatively high molecular weight, as compared to conventional compositions comprising such ingredients. Thus, the present nano-carriers may be used to prepare pharmaceutical and/or agrochemical products capable of delivering active agents having a relatively high MW and/or at a relatively high concentration. Without wishing to be bound by theory, the relatively higher loading of the WITC(s) and/or active agent(s), as well as the relatively higher potency of the nano-elements (when the efficacy also depends on the MW of the active agent(s) that may be delivered) are each expected to improve the products' efficacy (be it pharmaceutic or agricultural).

In a second aspect of the disclosure, there are provided uses of a composition for the preparation of a medication for the treatment of a living subject or of an object by administration of the medication through non-exposed surfaces (e.g., by ND routes) of said subject or object, the composition comprising nano-elements dispersible in a polar carrier, the nano-elements being core-shell nano-elements composed of:
I. a core comprising at least one WITC;
II. a shell surrounding the core, the shell comprising at least one polar-carrier-insoluble shell-forming agent (SFA) or a portion thereof; and
III. a polar-carrier-insoluble active agent disposed in at least one of the core and the shell of the nano-elements;
   wherein each constituent of the core-shell nano-elements has a vapor pressure of 40 Pascal (Pa) or less, as measured at a temperature of about 20° C.; and wherein the nano-elements have an average diameter (e.g., D_{N}50) of 1,000 nm or less.

As far as the materials are concerned, the WITC and the polar carrier of the core-shell nano-elements, and to the extent present in the composition a carrier-insoluble active agent, a non-volatile liquid, a surfactant, and a carrier-soluble active agent, are substantially as described above and herein detailed. As the methods for preparing core and core-shell(s) nano-elements share common steps, the core is believed to be continuous/non-porous in any case.

All materials forming the core or the core-shell nano-elements according to the first or second aspect herein disclosed, have been set to be relatively non-volatile as expressed by their individual or combined vapor pressure not exceeding 40 Pa. In some embodiments, the vapor pressure of the nano-elements and of each of their constituents is 20 Pa or less, 5 Pa or less, or 1 Pa or less, as measured at a temperature of about 20° C.

In some embodiments, the SFA is not covalently bonded to the WITC.

Referring to the shell-forming agents generating a shell-like structure surrounding the core when mixed with the WITC(s) during the preparation of the nano-elements, they are generally amphiphilic molecules, composed of a relatively large hydrophobic part, forming a hydrophobic “tail”, and of a hydrophilic “head”, wherein the hydrophobic tails are entrapped within the core containing the WITC and the hydrophilic heads, under suitable conditions (e.g., viscosity of the core, polarity of a surrounding liquid), migrate to the surface of the core, forming the shell.

The hydrophobic tail of the SFA that can be predominantly found within the core of the core-shell nano-elements is typically the fatty portion of a fatty compound, the tail usually being a straight, branched, or cyclic, saturated or unsaturated, aliphatic or aromatic, alkyl or aryl hydrocarbon chain. In some embodiments, the water-insoluble/WITC-miscible SFA forming the shell is selected from the group comprising: fatty amines, fatty acids, and metal salts of alkyl aryl sulfonates or of petroleum sulfonates. When the SFA is a fatty amine, the hydrocarbon chain generally contains between 8 and 22 carbon atoms. When the SFA is a fatty acid, the chain usually contains between 5 and 40 carbon atoms. When the SFA is a metal salt of a sulfonate, the metal counterion can for example be barium, calcium, magnesium or sodium, and the alkyl aryl or petroleum hydrocarbon chain usually contains between 20 and 30 carbon atoms.

In contrast with other molecules that may be added to the WITC(s) and may inherently migrate during the preparation of the nano-elements, the hydrophilic head of the SFA that can be predominantly found in the shell of the core-shell nano-elements is typically a chemical group chargeable in the polar carrier in which the core-shell nano-elements are dispersible. Depending on the chargeable chemical group and on the polar carrier (e.g., composition and pH), the shell may be positively chargeable or negatively chargeable, the overall charge of the core-shell nano- elements in the same polar carrier furthermore depending on the relative proportion of SFAs to WITCs, and on the chemical identity of the WITCs, which typically provides for a negative charge. Thus, depending on the chemical identity and respective amounts of the materials constituting the core and the materials constituting the shell, when present, as well as the medium in which such core or core-shell nano-elements are dispersed, they can be tailored to have a positive or a negative charge having an absolute value of 5 mV or more, 10 mV or more, 20 mV or more, 30 mV or more, or 40 mV or more, the absolute value of the charge typically not exceeding 100 mV. For illustration, a negative charge can be in the range of-100 mV to-5 mV and a positive charge can be in the range of +5 mV to +100 mV. Such charge or chargeability of the nano-elements, in presence or absence of a shell, is typically assessed in presence of water and measured at room temperature.

The relative charge of the nano-elements may facilitate their dispersibility in a liquid medium and/or the attachment of additional molecules (e.g., and/or to desired target tissues). Nano-elements sufficiently charged (positively (e.g., by +30 mV or more) or negatively (e.g., by at least −30 mV)) may be stably dispersible by virtue of the electrostatic repulsive forces between the charged nano-elements so as to preclude the need for dedicated dispersants. Compositions containing such nano-elements, self-sufficient with respect to dispersibility, can be said to be “self-emulsified”, “self-emulsifiable” and like grammatical variants. Nano-elements having a charge between −30 mV and +30 mV may require particular attention to remain stably dispersed, if the medication is in liquid form, and optionally suitable modifications of the liquid medium (e.g., by inclusion of dispersants or any other agents favoring repulsion of the nano-particles by mechanisms other than electrostatic). However, the chargeability of the nano-element is not essential and the core or the core-shell nano-elements may have a near neutral charge between −5 mV and +5 mV.

In particular embodiments, the water-insoluble/WITC-miscible SFA is selected from a group consisting of oleyl amine, octyl amine, oleyl bis-(2-hydroxyethyl) amine, N,N-dimethyl-dodecylamine (DMDA), cetrimonium chloride (cetyl-trimethyl-ammonium chloride (CTAC)), caprylic acid, and combinations thereof.

In some embodiments of the second aspect, the compositions used in the present invention further contain in the polar carrier a water-soluble active agent, this material forming a second shell on the surface of the core-shell nano-elements. In some embodiments, the second shell is composed of at least one layer of the carrier-soluble active agent. Nano-elements comprising a) a core of WITC including at least WITC-miscible/polar-carrier-insoluble active agent(s) (and optionally non-volatile liquid(s),surfactant(s) and like WITC-miscible/polar-carrier-insoluble materials as may be desired in the core), b) a first shell including at least a polar portion of the SFA(s) and c) a second shell being at least one layer of a carrier-soluble active agent, can be referred to as core-shells or as core-multi-shells nano-elements. As used herein, the term core-shell(s) encompasses core-shell nano-elements having only a first shell including SFAs directly surrounding the cores and core-shells nano-elements further having a second shell surrounding the first one and indirectly surrounding the core.

Advantageously, the first shell of a core-multi-shells nano-element, which is charged in an aqueous polar carrier of the composition, facilitates the non-covalent formation of the second shell. For instance, a positively-charged first shell can electrostatically attract molecules of carrier-soluble active agents having a negative charge in the polar carrier. In such a case, the nano-elements, which were formerly positively charged with a single shell of a SFA in the medium being considered, may become less positively charged, electrostatically neutral, or negatively charged. Additionally, or alternatively, the first shell can form covalent bonds with the second shell of carrier-soluble active agents facilitating their indirect attachment to the core.

Preferably, the covalent or non-covalent wrapping of the second shell around the first shell is such that the nano-elements do not increase in size beyond the size ranges herein disclosed.

As an active agent suitable for the preparation of the present medications can be apolar and fully miscible in the WITC(s), polar and fully soluble in the polar carrier, or amphiphilic so that in analogy with the SFA its hydrophobic part could be within the core and it hydrophilic part forming a shell surrounding the core, the active agent can respectively be said to be (entirely) contained in the core, (externally) anchored to the core (e.g., covalently or electrostatically via a first shell) or (partially) entrapped within the core (i.e., by way of a hydrophobic tail). If more than one active agent is included in the present nano-elements, they may also be disposed in different areas of the core-shell(s).

With respect to the uses of compositions as herein disclosed for the preparation of a medication, and the treatments of a living subject or of an object they may enable following the administration of said medications through non-exposed surfaces (e.g., by ND routes), they depend upon the active agent being contained in, entrapped by and/or anchored to the core of the nano-elements. If the subjects due to benefit from such medications or treatment are human persons or non-human animals, such uses can be for the preparation of a medication being a pharmaceutical product (including a veterinary product), such medications being configured for the treatment (including any step medically relevant from diagnosis to cure, via prevention, delay or reduction, etc.) of any ailment according to the active agent(s) present in the nano-elements, regardless of the part of the nano-elements in which they are disposed.

If the non-exposed surfaces are not of subjects to be treated but of objects of relevance to human or veterinary medicine, the use of the nano-elements may still be considered pharmaceutic. For illustration, the present medications may serve to coat prosthetic implants intended to artificially replace a part of the body. Alternatively, the nano-elements can be used for the preparation of agrochemical products, the objects being plants or parts thereof.

Additional objects, features and advantages of the disclosure will be set forth in the detailed description which follows, and in part will be readily apparent to those skilled in the art from the description or recognized by practicing the disclosure as described in the written description and claims hereof, as well as the appended drawings. Various features and sub-combinations of embodiments of the disclosure may be employed without reference to other features and sub-combinations.

For illustration, additional objects include the corresponding methods of treatment of a condition treatable by the active agent(s) that can be delivered by the present nano-elements, following the administration of an effective amount of said agent(s) to a subject or object in need of said treatment, and the medications that may be delivered in such methods.

### BRIEF DESCRIPTION OF THE DRAWINGS

Some embodiments of the disclosure will now be described further, by way of example, with reference to the accompanying figures, where like reference numerals or characters indicate corresponding or like components. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the disclosure. For the sake of clarity and convenience of presentation, some objects depicted in the figures are not necessarily shown to scale.

In the Figures:

FIG. 1 depicts a simplified schematic diagram of a method for preparing nano-elements suitable for a composition according to an embodiment of the present teachings;

FIG. 2A shows the particle size distribution of core nano-elements comprising a WITC-miscible active agent, prepared according to the present teachings, as measured by dynamic light scattering and presented per number;

FIG. 2B shows the particle size distribution of core-shell nano-elements comprising a WITC-miscible active agent and an SFA, prepared according to the present teachings, as measured by dynamic light scattering and presented per number;

FIG. 3 schematically illustrates a core-shell nano-element wherein the polar portion of the SFA forming a first is uncharged (e.g., in a non-aqueous polar carrier);

FIG. 4 schematically illustrates a positively charged core-shell nano-element, surrounded by a second shell formed of molecules of an active agent;

FIG. 5 is a CryoTEM image of core nano-elements, prepared according to the present teachings safe for the inclusion of a WITC-miscible/polar-carrier-insoluble active ingredient;

FIG. 6A is a CryoTEM image of core-shell nano-particles surrounded by a second shell of a carrier-soluble active agent, the nano-particles being prepared according to the present teachings;

FIG. 6B is a CryoTEM image of core-shell nano-particles similar to the ones shown in FIG. 6A, surrounded by a second shell which have undergone partial coalescence;

FIG. 7A is a picture of cortical neuronal cells following the application of nano-elements according to an embodiment of the present teachings, the core including a fluorescent marker to demonstrate cellular penetration;

FIG. 7B is a schematic depiction of the cellular penetration image of FIG. 7A; and

FIG. 8 is a pharmacokinetic plot showing the changes in the concentration of tadalafil in rats' plasma over time following oral administration of nano-elements according to the present teachings comprising tadalafil in their core, as compared to similar doses of commercial Cialis® and bulk tadalafil provided as suspensions.

### DETAILED DESCRIPTION

The present invention relates inter alia to uses of compositions for the preparation of pharmaceutical products (for the treatment of human and non-human subjects) and agrochemical products (for the treatment of objects) by administration via non-exposed surfaces of said subjects or objects, the compositions comprising nano-elements, e.g., nano-particles or nano-droplets, containing water-insoluble thermoplastic compound(s) or WITC(s) (in particular, water-insoluble thermoplastic polymer(s) or WITP(s)) capable of forming a core, the nano-elements further containing active agent(s), and being dispersible or dispersed as a nano-suspension in a polar carrier. The nano-elements can be in the form of core or core-shell(s) nano-elements as described above and herein detailed. Advantageously, the WITC can be, if desired, plasticized by a non-volatile liquid, which can also be referred to as a plasticizing agent. As the nano-elements can be prepared with constituents having a low volatility, they may be characterized by a low content of volatile organic compounds (VOC(s)), such volatile compounds being present in an amount of less than 1 wt. % of VOC, or a blend thereof, by weight of the nano-elements. The nano-elements comprising the optionally plasticized WITC(s) and the active agent(s) may further include any other desired compound, such as any material miscible with the cores or any of the shells (for core-shell(s) nano-elements) to enable or increase the dispersibility of the nano-elements in the composition, to modulate their charge or any other property adapted to increase the stability of the nano-elements over time, to further enhance or modify the activity of the composition (e.g., the release of the active agent(s) from the nano-elements) and/or to increase the compatibility of the nano-elements with their intended formulation as a medication. Methods for preparing such nano-elements are also disclosed.

Before explaining at least one embodiment in detail, it is to be understood that the disclosure is not necessarily limited in its application to the details of construction and the arrangement of the components and/or methods set forth herein. The disclosure is capable of other embodiments or of being practiced or carried out in various ways. The phraseology and terminology employed herein are for descriptive purpose and should not be regarded as limiting.

It is to be understood that both the foregoing general description and the following detailed description, including the materials, methods and examples, are merely exemplary of the disclosure, and are intended to provide an overview or framework to understanding the nature and character of the invention as it is claimed, and are not intended to be necessarily limiting.

### Biodegradability

The WITCs used in the present invention can be biodegradable, and as such, broken down by any suitable mechanism of biodegradation adapted to their structure and chemical identity. Upon the delivery of nano-elements following their administration to a living subject or an object, the WITCs can be degraded by biological mechanisms so that the active agent(s) carried thereby can be released at the site and time of biodegradation and over its duration. Suitable WITCs are typically biocompatible with the physiological environment in which they may biodegrade, such as found following their delivery in the form of nano-elements. Suitable WITCs can also be termed bioresorbable or bioabsorbable in the general literature, depending on their in vivo fate and prospective elimination from the body, but for simplicity all such compounds will be generally referred to herein as “biodegradable”. A WITC is said to be biodegradable if breaking down relatively rapidly after fulfilling its purpose (e.g., by a bacterial decomposition process in the environment or by an in vivo enzymatic or metabolic process) to result in natural by-products. Biodegradable WITCs are known, and new ones are being developed. Their relative biodegradability in various environments can be assessed by a number of methods, which depending on the conditions of interest can be procedures based on or modified from standards such as ASTM F1635.

Despite its propensity to naturally decompose under suitable physiological conditions, a biodegradable WITC adapted to the present invention should be stable and durable enough for its intended use during storage and administration until the nano-elements reach the site to be targeted by the active agents carried thereby, which can be particularly challenging if this use involves conditions that would enhance biodegradability. For example, compositions containing the WITCs, formulated as a tablet and administered orally, are expected to endure the passage through the digestive system, and such medications should avoid the degradation of the WITCs before they reach their target. Hence, the selection of suitable WITCs for the pharmaceutical and agrochemical compositions used in the present invention should take these factors into consideration.

The WITCs used in the compositions can also be biocompatible, operating without eliciting undesired local or systemic effects in the subject or object administered thereto. Standard methods for assessing biocompatibility are known, such as the ones specified in ASTM F748 and ISO 10993.

### Insolubility

Besides their possible biodegradability and biocompatibility, the WITCs are preferably substantially non-soluble in the liquid phase of the composition including the polar carrier (e.g., water), in which they are dispersible as nano-elements during their preparation. For similar reasons, they should be substantially insoluble in the liquid vehicle of a liquid dosage form (whether same or different than the polar carrier used during their preparation).

As used herein, the solubility of a material (e.g., a WITC, a non-volatile liquid, or an active agent) refers to the amount of such component that can be introduced into the liquid (e.g., polar) carrier, while maintaining the clarity of the liquid medium. The solubilities of specific components of the composition within any particular liquid are typically assessed in the sole polar carrier in absence of any other possible components of the compositions but may be alternatively determined with respect to the final composition of the liquid phase including the carrier or with respect to the final composition of the liquid vehicle of a liquid dosage form.

WITCs (or any other material of interest for the present invention) are deemed insoluble if their solubility in the polar carrier, or in the liquid phase containing it, or in the liquid vehicle of a dosage form, is 5 wt. % or less, 4 wt. % or less, 3 wt. % or less, 2 wt. % or less, 1 wt. % or less, 0.5wt. % or less, or 0.1 wt. % or less by weight of the fluid being considered. For illustration, no more than 5 g of a material that is non-soluble in a polar carrier would dissolve in 100 g of the carrier. This substantial insolubility, while typically measured at room temperature, should preferably apply at any temperature at which these ingredients are combined and processed, i.e., even at relatively elevated temperatures, the solubility of these compounds in the polar carrier should remain within the required ranges. A material satisfying these conditions can be referred to as a “polar-carrier-insoluble” material, when the liquid environment considered is a polar carrier.

Such insolubility of the material is expected to prevent or reduce leaching out into their surrounding media of one or more of the WITC and of any other one of the WITC-miscible constituents of the nano-elements (e.g., carrier-insoluble active agents, non-volatile liquids, or SFAs). Such leaching out, were the material soluble in the polar carrier, may affect the relative proportions of the constituents of the nano-elements, their size, or any other such parameter that may ultimately adversely affect the efficacy of the medication. Considering for illustration an active agent initially embedded in the nano-elements of WITCs, if such agents were to leak out in an uncontrolled manner as a result of inappropriate solubility, such agents could be discharged from the nano-carriers in an untimely manner and/or possibly at an inadequate localization with respect to a desired target site.

Regardless of the composition of the polar liquid phase including the polar carrier in which the nano-elements are dispersible, the polar-carrier insoluble WITCs, active agents or SFAs (and any like material miscible with the WITCs) can first be characterized as being water-insoluble (i.e., having a solubility of less than 5 wt. % in water as typically established at room temperature).

### Molecular Weight

Advantageously, the present invention allows for the delivery of nano-elements comprising WITCs having relatively high molecular weights as compared to compounds that may conventionally sufficiently penetrate through biological barriers (such as blood vessel walls or cell membranes) to display any efficacy. WITCs suitable for the present compositions, methods, and uses can have a molecular weight (MW) of 0.6 kDa or more, 0.7 kDa or more, 0.8 kDa or more, 0.9 kDa or more, 1 kDa or more, 1.5 kDa or more, WITPs also displaying MW of 2 kDa or more, 2.5 kDa or more, 3 kDa or more, 3.5 kDa or more, 4 kDa or more, 4.5 kDa or more, 5 kDa or more, 5.5 kDa or more, 6 kDa or more, 6.5 kDa or more, 7 kDa or more, or 10 kDa or more. Typically, their molecular weight does not exceed 2 kDa if the compound is not a polymer, WITPs reaching MWs of up to 500 kDa, and being generally of 300 kDa or less, 200 kDa or less, 100 kDa or less, 80 kDa or less, 50 kDa or less, 25 kDa or less, or of 15 kDa or less. In another embodiment, the molecular weight of the WITCs is between 0.6 kDa and 500 kDa, between 0.7 kDa and 300 kDa, between 0.8 kDa and 200 kDa, between 1 kDa and 100 kDa, between 2 kDa and 80 kDa, between 1.5 kDa and 500 kDa, between 2.5 kDa and 300 kDa, between 3 kDa and 200 kDa, between 3.5 kDa and 500 kDa, between 4 kDa and 500 kDa, between 5 kDa and 300 kDa, between 5.5 kDa and 300 kDa, between 6 kDa and 200 kDa, between 6.5 kDa and 200 kDa, or between 7 kDa and 200 kDa.

As used herein, the term “molecular weight” (or “MW”) refers either to the actual molecular weight as can be calculated for a non-polymeric WITC or for any other compound having a known molecular structure, which can also be expressed in grams/mole, or to the weight average MW of polymerizable WITCs or WITPs, which may be a blend of polymers each containing a slightly different number of repeating units, weight average MW of polymers being typically expressed in Daltons.

The molecular weight of the WITCs can be provided by their suppliers and can be independently determined by standard methods including for instance gel permeation chromatography, high pressure liquid chromatography (HPLC), size-exclusion chromatography, light scattering or matrix-assisted laser desorption/ionization time-of-flight mass spectroscopy MALDI-TOF MS, some of these methods are described in ASTM D4001 or ISO 16014-3.

Such general rules concerning the molecular weight exemplified with respect to the WITCs can be applied to any other constituent of the composition, whether polymeric or not, and in some embodiments, active agents added to the cores and/or the shells of the nano-elements may also be relatively large molecules having a MW of 0.6 kDa or more, and any other values as specifically recited for the WITCs. While most active agents are relatively small molecules (most being smaller than 1 kDa and many having a MW of less than 0.6 kDa), numerous important ones are relatively large. This would be the case for instance for biologics or biopharmaceuticals, which are typically derivatives of naturally occurring compounds (e.g., proteins, polysaccharides, nucleic acids, etc.). Example of relatively large active agents include the hormone insulin (˜6 kDa), the polysaccharide hyaluronan, vaccines, blood factors, enzymes, antibodies or parts thereof, adalimumab (Humira®) having a MW of about 144 kDa.

### Characterizing Temperatures

While the vast majority of non-polymeric compounds can be characterized by a melting temperature (Tm) at which they change from a solid phase to a liquid one, polymeric compounds can additionally or alternatively be defined by a glass transition temperature (Tg) if amorphous, pure amorphous polymers lacking a Tm. Pure crystalline polymers can be characterized by their Tm, semi-crystalline polymers often displaying two characterizing temperatures (e.g., Tg and Tm) reflecting the respective proportion of amorphous and crystalline parts in the molecule. Such polymers may also be defined by their softening temperature (Ts) midway the log step to melting. As the glass transition temperature describes the transition of a glass state into a rubbery state, and the softening temperature an intermediate inflection in the thermal analysis of a material, they typically relate to a range of temperatures or one at which the process will first be observed.

Therefore, depending on the chemical nature of the WITC, the temperature that may characterize its thermal behavior can be at least one of a Tm, a Ts and a Tg. Hence, when a WITC (or nano-elements comprising a WITC) is defined as suitably having at least one of a first (or second) Tm, Ts and Tg within a particular range, the temperature considered is as relevant to the material. Some compounds may be identified by two such characterizing temperatures, in which case performing a method step at a temperature above any of the two temperatures could be above the lowest of the two (which would prolong the step) or the highest of the two (which would accelerate the step). Conversely, performing a method step at a temperature below any of the two temperatures could be below the highest of the two or the lowest of the two. Taking for illustration a semi-crystalline polymer that can be characterized by all three temperatures, Tm, Ts, and Tg in order of decreasing values, heating above Tg (i.e., above at least one), might be insufficient to reach Ts or Tm, while heating above Ts (i.e., above at least two), might be insufficient to reach Tm. Only heating above Tm would ensure that the temperature of heating is higher than all three temperatures that may characterize such an exemplary polymer.

In some embodiments, the WITCs suitable for the present compositions are characterized by at least one of a first (native) melting temperature (Tm), softening temperature (Ts) or glass transition temperature (Tg) being of at most 300° C., at most 250° C., at most 200° C., at most 190° C., at most 180° C., at most 150° C., or at most 120° C.

In some embodiments, the first Tm of the WITCs is at least 0° C., at least 10° C., at least 20° C., at least 30° C., at least 40° C., at least 50° C., or at least 60° C. In other embodiments, the first Tm of the WITCs is between 0° C. and 300° C., between 10° C. and 300° C., between 20° C. and 300° C., between 20° C. and 250° C., between 20° C. and 200° C., between 30° C. and 180° C., between 40° C. and 150° C., or between 50° C. and 120° C.

In some embodiments, the WITCs are characterized by at least one of a first Ts and a first Tg being of-75° C. or more,-50° C. or more,-25° C. or more, 0° C. or more, 10° C. or more, 20° C. or more, 25° C. or more, 30° C. or more, 40° C. or more, 50° C. or more, or 60° C. or more. In some embodiments, at least one of the first Ts and Tg of the WITCs is between −75° C. and 300° C., between −50° C. and 250° C., between −25° C. and 200° C., between 0° C. and 180° C., between 20° C. and 300° C., between 20° C. and 250° C., between 20° C. and 200° C., between 20° C. and 180° C., between 30° C. and 180° C., between 40° C. and 180° C., between 30° C. and 150° C., between 50° C. and 150° C., or between 50° C. and 120° C.

The characterizing temperatures (Tm, Ts or Tg) of a WITC may be referred to as a “first” Tm, Ts or Tg, when relating to the native/unmodified compound, and may be referred to as a “second” Tm, Ts or Tg, when relating to the WITC as modified to constitute the cores of the present nano-elements. In some embodiments, the second Tm, Ts or Tg that can be measured on the blend obtained upon mixing of the WITC with other components, such as: a WITC-miscible active agent, a shell-forming agent (SFA), a non-volatile liquid or any other desirable ingredient, or rather, measured on the nano-elements containing the same, is similar to the first respective Tm, Ts or Tg of the WITC. Alternatively, the second Tm, Ts or Tg can be lower than the respective first characterizing temperature(s) of the WITC, in which case the WITC is considered plasticized or swelled.

Such general rules concerning a suitable thermal behavior exemplified with respect to the WITCs can be applied to any other constituent of the nano-elements which may display or affect a characterizing temperature as herein described. Hence, in some embodiments a constituent of the nano-elements (other than the WITCs), either alone or in combination with the WITC and all other materials due to form the nano-elements (e.g., active agents, SFAs, etc.) may satisfy the aforesaid ranges. In other words, the aforesaid thermal properties of the bulk, native, WITCs (e.g., a first Tm, a first Ts, or a first Tg and the values they should preferably satisfy) can be read to similarly apply to the nano-elements and their respective second Tm, second Ts or second Tg.

Such thermal characteristics of a WITC or any other ingredient can be provided by its manufacturer or independently determined by standard methods, in particular for the nano-elements. Thermal analysis methods, e.g., Differential Scanning calorimetry (DSC), are described, for instance, in ASTM 3418, ISO 3146, ASTM D1525, ISO 11357-3, or ASTM E1356. Such measurements may be performed on the raw materials (e.g., bulk WITC) or on intermediate (e.g., a mix) or end-products (e.g., the nano-elements) containing the WITC. For this purpose, the nano-elements containing the WITC may be isolated from the polar carrier and other agents present therein. The separation of the nano-elements from their liquid medium can be performed according to standards methods, such as by evaporation of the carrier by drying (e.g., in a vacuum oven) or freeze-drying, or by destabilization of the composition (e.g., by changing the pH) allowing the precipitation of the nano-elements. Nano-elements isolated by any suitable method from their samples can be additionally rinsed (e.g., with water) to remove residues that may affect the intended measurements, the nano-elements being then separated from the rinsing liquid. For instance, nano-elements precipitated out of their initial medium can be separated by centrifugation, the precipitate can be rinsed and centrifuged again in cycles until isolated and desirably rinsed nano-elements are obtained.

### Polymeric and Non-Polymeric WITCs

In some embodiments, the water-insoluble thermoplastic compounds (WITCs) used according to the present teachings are water-insoluble thermoplastic polymers (WITPs), such polymers also including their fragmented/shorter versions known oligomers. As the WITPs are desirably adapted for biodegradation once delivered into the physiological environment of the living subject or object, such polymers generally contain hydrolysable functional groups or enzymatically cleavable sites. The presence of hydrolysable or otherwise cleavable sites is however non-essential, and polymers considered non-biodegradable may lack them.

In some embodiments, the WITCs (or WITPs) may be non-reactive and unable to build up more complex interactions, being only able to biodegrade, such as polycaprolactone. In other embodiments, the WITCs (or WITPs) may be biodegradable and have reactive moieties enabling interactions with additional different molecules, such as polylactic acid, or additional same molecules, such as polymerizable natural resins having for instance aldehyde moieties. Additionally, the WITCs (or WITPs) may be modified, e.g., by chemically binding functional groups, to provide for, enhance or modulate their properties.

Similarly, when the WITCs are non-biodegradable compounds, such materials can also be reactive or not. Non-reactive non-biodegradable synthetic thermoplastic polymers can be for illustration polyethylene or polypropylene polymers, while polymers containing reactive groups can be e.g., ethylene-acrylic acid or ethylene-methacrylic acid copolymers.

As suitable WITPs, which can be of natural or synthetic origin, are thermoplastic in nature, their shapes are capable of reversible modifications upon suitable heating and cooling. Appropriate WITPs can also be plasticized with a suitable non-volatile liquid, such treatment of the WITPs facilitating their nano-sizing to an extent expediting the delivery of the nano-elements and the active agents contained therein or carried thereby.

Synthetic WITPs can be biodegradable and selected from aliphatic polyesters, polyhydroxy-alkanoates, poly(alkene dicarboxylates), polycarbonates, aliphatic-aromatic co-polyesters, enantiomers thereof, copolymers thereof and combinations thereof.

To the extent that the monomers forming the WITPs have chiral centers, all enantiomers and stereoisomers are encompassed. For illustration, lactic acid (2-hydroxypropionic acid, LA), exists as two enantiomers, L-and D-lactic acid, so that PLA has stereoisomers, such as poly(L-lactide) (PLLA), poly(D-lactide) (PDLA), and poly(DL-lactide) (PDLLA). A WITP may therefore be a mixture of isomers of a same molecule or a specific stereoisomer (or a stereo copolymer).

In some embodiments, the WITP is biodegradable and selected from a group comprising: aliphatic polyesters, such as polycaprolactone (PCL), polylactic acid (PLA), poly(L-lactide) (PLLA), poly(D-lactide) (PDLA), poly(D,L-lactide) (PDLLA), polyglycolic acid (PGA), poly(lactic-co-glycolic acid) (PLGA), and poly(p-dioxanone) (PPDO); polyhydroxy-alkanoates (PHA), including polyhydroxybutyrate (PHB) (such as poly-3-hydroxy-butyrate (P3HB), poly-4-hydroxy-butyrate (P4HB), poly(3-hydroxybutyrate-co-3-hydroxyvalerate) (PHBV), poly-hydroxyvalerate (PHV), polyhydroxyhexanoate (PHH), and polyhydroxyoctanoate (PHO); poly(alkene dicarboxylates), such as poly(butylene succinate) (PBS), poly(butylene succinate-co-adipate) (PBSA) and poly(ethylene succinate) (PES); polycarbonates, such as poly-(trimethylene carbonate) (PTMC), poly(propylene carbonate) (PPC) and poly-[oligo (tetramethylene succinate)-co(tetramethylene carbonate)]; aliphatic-aromatic co-polyesters, such as poly(ethylene terephtalate) (PET) and poly(butylene adipate-co-terephtalate) (PBAT); their isomers, copolymers and combinations thereof.

In a particular embodiment, the biodegradable WITP is or includes an aliphatic polyester, isomers, copolymers and combinations thereof. In a further particular embodiment, the WITP is PCL. In another further particular embodiment, the WITP is PLA. In a further particular embodiment, the WITP is PLGA. In another further particular embodiment, the WITP is PBSA.

Natural biodegradable WITPs can be selected from polysaccharides (such as: cellulose, starch, chitin and chitosan), lignin and combinations thereof.

In some embodiments, the WITP is non-biodegradable and selected from polyamide (PA), polyethylene (PE), poly(ethylene-co-acrylic acid) (PEAA), poly(ethylene-co-methacrylic acid) (PEMAA), poly(ethylene-co-n-butyl acrylate) (PEBA), poly(ethylene-co-vinyl acetate) (PEVA), polymethylmethacrylate (PMMA), polypropylene (PP), polysiloxane, polystyrene (PS), polytetrafluoroethylene (PTFE), polyurethane (PU), or polyvinyl chloride (PVC). In a particular embodiment, the non-biodegradable WITP is selected from PA, PE, PEAA, PEMAA, PEBA, PEVA, substituted or modified versions thereof, ionomers thereof and combinations thereof.

The above-mentioned polymers may be identified according to their respective characteristic functional groups as detectable by standard methods known to the skilled persons, for instance, by Fourier-transform infrared (FTIR) spectroscopy.

Non-polymeric WITCs suitable for the compositions, methods and uses of the present invention can also be natural or synthetic. Under suitable conditions, some natural WITCs may react in a polymerization reaction, resulting in WITPs.

Natural WITCs that may be polymerizable can be selected from: natural resins (such as: shellac and vegetal resins including amber resin, colophony resin (also known as rosin), copal resin, dammar resin, dragon blood resin, elemi resin, kauri resin, mastic resin, manila resin, sandarac resin, tacamahac resin and fractions thereof); natural gums (either originating from gum-yielding trees, such as Acacia nilotica (babul), Acacia catechu (khair), Steruculia urens (kullu), Anogeissus latifolia (dhawra), Butea monosperma (palas), Bauhinia retusa (semal), Lannea coromandelica (lendia) and Azadirachta indica (neem), or originating from seeds of plants such as guar, tamarind and Cassia tora); natural gum-resins (such as asafoetida, myrrh, salai and guggul); waxes (such as: candelilla wax, carnauba wax, jojoba wax, kafrin wax, montan wax, olive wax, rice bran wax, sal tree wax, sugarcane wax, sunflower wax, and fractions thereof); and combinations thereof. In a particular embodiment, the natural polymerizable WITC is shellac or gum rosin.

Non-polymerizable WITCs include quinones. In a particular embodiment, the non-polymerizable WITC is coenzyme Q10 (CoQ10).

Additionally, a WITC, can be a blend of different compounds, whether polymeric, polymerizable, or not, the properties of the mixture (e.g., a characterizing temperature, a viscosity, etc.) satisfying the ranges set for a suitable individual compound. For instance, a WITC or WITP having a Tm, Ts or Tg out of a range previously deemed suitable (e.g., being lower than 20° C. or higher than 300° C.) may be combined with a WITC or WITP having a Tm, Ts or Tg adapted to “correct” the characterizing temperature of the obtained mixture to be suitable for the purpose of the present invention. For illustration, a WITC can be a blend of polymers or a copolymer including at least one of the aforementioned WITPs, such copolymers may contribute to the biocompatibility, biodegradability and mechanical and optical properties of the nano-elements.

The compositions used in the present invention may contain nano-elements each prepared using various types of WITCs. For instance, a composition may include nano-elements prepared using one type of WITC combined with nano-elements prepared using a different type of WITC. Alternatively, or additionally, the present compositions can contain nano-elements which differ in their type (core or core-shell), function (for instance each type of nano-elements including a different active agent, resulting in a combined activity) and/or viscosity (for instance each type of nano-elements being capable of releasing a same or a different active agent at different onsets and/or rates, resulting in an activity over an extended period of time). Viscosity

Without wishing to be bound to any particular theory, it is believed that the viscosity of the nano-elements may assist controlling the release profile of an active agent contained or entrapped in the core, or otherwise attached to it. Simply put, it is expected that nano-elements having a relatively lower viscosity will more readily and quickly release the active agent than nano-elements having a relatively higher viscosity. As the release of the active agent can be accordingly tailored to be relatively fast or relatively slow (the rate of release not necessarily being constant during the release period), it may be advantageous, in cases for which a prolonged duration of release is desired, to include in the medication nano-elements spanning a wide range of viscosities, each portion of the range of viscosities providing for a release of the active agent within a respective portion of the release window. Alternatively, if for a particular treatment the onset and duration of release is to be more specific, the viscosity of the nano-elements can be adapted to such narrower purposes and selected accordingly. The viscosity that may be suitable for a particular onset, rate, peak and/or duration of release may inter alia depend on the composition of the nano-elements (e.g., type of WITC and/or contents of plasticizing materials) and on the environment targeted by the medication, and its impact on the degradation of the nano-elements. Such parameters can be experimentally assessed, for instance by monitoring the release of the active agent of interest from intended nano-elements prepared to have a series of pre-determined viscosities, the release being monitored while the nano-elements are incubated in a medium and at a temperature best mimicking the conditions under which the medication is due to operate.

Accordingly, WITCs can be selected for their viscosity to be adapted to the desired release profile of the active agent(s) from the nano-elements. WITCs suitable for the present uses and the method by which the nano-elements can be prepared typically have a viscosity which does not exceed of 10¹¹ millipascal-second (mPa's, being equivalent to a centipoise), and which is often of 5×10¹⁰ mPa·s or less, 10¹⁰ mPa·s or less, 5×10⁹ mPa·s or less, 10⁹ mPa·s or less, 5×1⁰⁸ mPa·s or less, 10⁸ mPa·s or less, 5×10⁷ mPa·s or less, 10⁷ mPa·s or less, or of 5×10⁶ mPa·s or less, as determined at at least one temperature between 20° C. and 80° C., and at a shear rate of 10 sec⁻¹.

It is to be noted that the temperature at which the viscosity of the WITCs (or of the nano-elements comprising the same) can be measured to assess suitability may depend on the native characterizing temperatures of the WITCs (i.e., first Tm, Ts or Tg) or on a second Tm, Ts or Tg characterizing the nano-elements. For instance, polycaprolactone, having a first Tm of about 60° C., may be plasticized to yield a second Tm of 50° C. or less, so that the viscosity of a plasticized PCL can be measured at about 50° C. A WITC or nano-elements having a relatively higher first or second Tm may require the viscosity measurement to be performed at a temperature higher than 50° C. Conversely, if the WITC or the nano-elements have a relatively lower first or second Tm their viscosity can be measured at a temperature lower than 50° C. Despite the wide ranges of characterizing temperatures of the present WITCs (or nano-elements prepared therewith), it is believed that at at least one temperature within 30° C., within 20° C. or within 10° C. from 50° C. (i.e., between 20° C. and 80° C., between 30° C. and 70° C., or between 40° C. and 60° C.) the WITCs or the nano-elements may display the disclosed dynamic viscosities.

When the viscosity relates to the native property of the isolated unmodified WITC, or of a blend of WITCs, it can be referred to as a “first viscosity”. When the viscosity relates to the WITC as modified by its mixing with materials miscible therewith, it can be referred to as the “second viscosity” of the mixture containing the WITC (or of the nano-elements). For illustration, the second viscosity can be of a WITP plasticized with a suitable plasticizing agent (e.g., a non-volatile liquid) as optionally further impacted by the presence of an active agent or an SFA. The viscosity of a material (whether modified or not by the presence of others) at any temperature of interest (or in a range thereof) can be determined by routine thermo-rheological analysis, such as described in ASTM D3835 or ASTM D440.

While a non-volatile liquid can be added to the WITC or WITP regardless of their native viscosity, such materials are typically used in the present compositions or methods of preparing the nano-elements when the WITC has a relatively high first viscosity, such as higher than 10⁷ mPa·s, as measured as aforesaid. The non-volatile liquid (which may also be referred to as a plasticizing liquid) is contained in the core of the nano-elements, the liquid being typically adsorbed or otherwise retained by the WITC.

Such “plasticizing” typically results in weight gain and/or a volume gain relative to the WITC own mass or volume in its native form. Such plasticizing of the WITCs renders the plasticized WITCs softer and more malleable, as demonstrated by the second viscosity being smaller than the first, facilitating the incorporation within the core of any desirable material miscible with the WITCs (e.g., carrier insoluble active agents and/or SFAs), their later nano-sizing to form nano-elements, and/or the migration of materials enabling the formation of a shell (when desired) around the cores of said elements. The resulting nano-elements may expedite the release of active agents contained or entrapped in the core and/or anchored thereto as compared to nano-elements lacking a non-volatile liquid.

Advantageously, the reduced viscosity should, in a first stage, be adapted to the shearing process (e.g., shearing equipment, shearing temperature, etc.) being elected to nano-size the plasticized WITC (e.g., a plasticized WITP) to prepare the present nano-elements, and ultimately capable of providing for a desired release profile of the active agents from the nano-elements (e.g., a WITC-miscible/polar-carrier-insoluble active agent being released from the core of the nano-elements as they degrade in their targeted site or a polar-carrier-soluble active agent being released from its anchoring to the core as its interaction therewith diminishes with the degradation of the nano-elements). For instance, the non-volatile liquid and its proportion relative to the WITC can be selected to lower the viscosity of the WITC by at least half-a-log, or at least one log, and so on, as might be required. For illustration, if the WITC has a first viscosity of 10⁸ mPa·s, a plasticizing agent and its amount would enable a reduction of half-a-log if the WITC so plasticized has a second viscosity of 5×10⁷ mPa·s, or (if in a higher amount or if alternatively selected to be a more potent agent) would enable a reduction of one log if the WITC so plasticized has a second viscosity of 10⁷ mPa·s, as measured at at least one temperature in a range of 20° C. to 80° C. and at a shear rate of 10 sec⁻¹.

In some embodiments, the first viscosity of the WITCs or the second viscosity of the WITCs plasticized with the non-volatile liquid (and/or of nano-elements comprising it) is between 10² mPa·s and 10⁷ mPa·s, between 5×10² mPa·s and 10⁶ mPa·s, between 5×10² mPa·s and 10⁵mPa·s, between 10³ mPa·s and 5×10⁴ mPa·s, or between 10³ mPa·s and 10⁴ mPa·s, as measured at at least one temperature between 20° C. and 80° C., and at a shear rate of 10 sec⁻¹. Viscosity can be measured with any suitable rheometer equipped with a spindle adapted to the intended range of viscosities at the appropriate shear rate.

### Plasticization of WITCs

While mentioned above for their effects on the viscosity of the WITCs, to the extent reducing it would be desired, the non-volatile liquids that may be incorporated with the WITCs in the nano-elements may fulfil additional functions. Plasticizing, in particular of WITPs, can be visually observed, as the polymer appears to be swollen at a temperature below melting. At higher temperatures, the effect of the non-volatile liquid (or any other agent having a plasticizing effect) can be detected via its plasticizing activity, which includes the ability to lower at least one of the temperatures characterizing the native WITCs.

Decreasing a characterizing temperature of the WITC, allows accordingly lowering processing temperatures at which the nano-elements of the compositions can be prepared. For illustration, while the WITC can have a first (native) Tm, Ts or Tg of 200° C. or less in absence of a suitable non-volatile liquid, the addition of such plasticizing agents may yield a plasticized WITC having a second (modified) Tm, Ts or Tg, lower than the first, the second temperature being for instance of 95° C. or less. The drop in temperature afforded by the presence of the non-volatile liquid need not be as dramatic as illustrated, obviously depending on the value of the first Tm, Ts or Tg of the native WITC, on the second Tm, Ts or Tg as may be desired to facilitate the preparation of the nano-elements and/or their later delivery, preferably on the boiling temperatures (Tb) of liquids which are to remain present in the composition (but not necessarily if the steps are brief enough and/or the liquids in excess in case some are boiled away), and/or on the concentration of the plasticizing agent with respect to the compound being plasticized.

The characterizing temperature of a WITC may be satisfactorily reduced solely by the presence of WITC-miscible active agent(s) or by SFA(s) (when added to the WITC and having an inherent plasticizing effect in addition to their intended predominant role). Alternatively, a non-volatile liquid may be combined with the WITCs in order to reduce (or further reduce) at least one of their characterizing temperatures.

Typically, the second Tm, Ts, or Tg of the plasticized WITC (together with any other ingredients miscible with it) is lower than the respective first Tm, Ts, or Tg of the bulk WITC, by at least 5° C., at least 10° C., at least 15° C., at least 20° C., at least 25° C., at least 30° C., at least 35° C., at least 40° C., at least 45° C., or at least 50° C.

If the mixture of WITC(s) with any one of the carrier-insoluble active agent(s), SFA(s) and non-volatile liquid(s) it may include, further comprises ingredients (e.g., rheological modifiers, surfactants, preservatives, or any like material which may have a plasticizing effect) that may impact the softening property of the resulting combination due to form the core, or be within, the nano-elements, then additionally and alternatively, the thermal characteristics deemed suitable for the present invention would apply to the entire mixture.

Hence, in some embodiments, the at least one of a second Tm, Ts or Tg of the nano-elements (be them core or core-shell nano-elements) is at most 290° C., at most 250° C., at most 200° C., at most 190° C., at most 180° C., at most 170° C., at most 150° C., or at most 120° C.

In some embodiments, the nano-elements have a second Tm of 0° C. or more, 10° C. or more, 20° C. or more, 30° C. or more, 40° C. or more, 50° C. or more, or 60° C. or more. In some embodiments, the second Tm of the nano-elements is within a range of 0° C. to 290° C., 10° C. to 290° C., 20° C. to 290° C., 10° C. to 250° C., 20° C. to 250° C., 20° C. to 200° C., 30° C. to 190° C., 50° C. to 170° C., 50° C. to 150° C., 30° C. to 180° C., 40° C. to 180° C., 40° C. to 150° C., 50° C. to 170° C., 50° C. to 150° C., or 50° C. to 120° C.

In some embodiments, the nano-elements (core or core-shell) have at least one of a second Ts and second Tg being −75° C. or more,-50° C. or more,-25° C. of more,-20° C. or more,-10° C. or more, 0° C. or more, 10° C. or more, 20° C. or more, 25° C. of more, 30° C. or more, 40° C. or more, 50° C. or more, or 60° C. or more. In other embodiments, the at least one of the second Ts and Tg measured on the nano-elements is within a range of-75° C. to 290° C.,-50° C. to 290° C.,-25° C. to 290° C.,-20° C. to 290° C.,-10° C. to 290° C., 0° C. to 290° C., 10° C. to 250° C., 20° C. to 200° C., 30° C. to 190° C., 30° C. to 180° C., 40° C. to 180° C., 50° C. to 170° C., 50° C. to 150° C., or 50° C. to 120° C.

Such thermal behavior and characterizing temperatures can be assessed while preparing the plasticized WITC or mixtures including the same, or upon completion of the preparation method of the composition, namely on the nano-elements that may be isolated therefrom.

### Non-Volatile Plasticizing Liquids

As explained above, controlling the release profile of an active agent contained or entrapped in the core of the nano-elements or anchored thereto can be facilitated by their viscosity, which may be adjusted inter alia by the presence of non-volatile liquids in the cores of the nano-elements. As such non-volatile liquids may also affect the temperatures characterizing the nano-elements, and in turn the temperatures at which drug release could be facilitated, it may be advantageous to select non-volatile liquids that can both lower the viscosity of the WITC(s) and lower at least one of its Tm, Ts and Tg, as previously separately discussed.

The selection of such materials can be also considered with a view of improving the processability of the WITCs, so as to facilitate the preparation and dispersion of the nano-elements within the polar carrier phase from which they might be later isolated (e.g., to transfer them to a different liquid vehicle adapted to a particular liquid dosage form or to prepare a dry dosage form). Advantageously, suitable non-volatile liquids improve the processability of the WITC under conditions suitable for its shearing into nano-particles, the shearing temperature causing initially the formation of nano-droplets. First, as implied by their names, agents adapted to plasticize a WITC according to the present teachings are liquid at the temperature at which the WITC is to be processed, namely at least at one of the temperatures of mixing with the WITC and of shearing. Such liquid agents can also be liquid at room temperature.

To ensure that their effect would perdure, the plasticizing liquids are preferably non-volatile. As used herein, the term “non-volatile”, as can be used with regards to a liquid that may plasticize the WITC, refers to liquids exhibiting a low vapor pressure, such as less than 40 Pascal (Pa, also Newton per square meter) at a temperature of about 20° C. In some embodiments, a non-volatile liquid (or any other ingredient for which a low volatility is preferred) can have a vapor pressure of 20 Pa or less, 5 Pa or less, 1 Pa or less, 0.1 Pa or less, or 0.01 Pa or less as measured at about 20° C. Such vapor pressure values are typically provided by the manufacturer of the liquid, but can be independently determined by standard methods, such as described in ASTM D2879, E1194, or E1782 according to the range of the vapor pressure. The low or substantially null volatility of the non-volatile liquids that may be used to plasticize a WITC should preferably be maintained at the highest temperature at which the plasticized WITC is processed. The use of such non-volatile liquids allows the WITCs to remain in their plasticized state, without the risk of evaporation or elimination of the liquids, even at high temperatures of preparing the nano-elements for the pharmaceutical and agrochemical compositions according to the methods herein described.

Suitable non-volatile liquids are also characterized by having a boiling point (Tbi) that is higher than room temperature, higher than body temperature, and higher than an elevated temperature as may be desired for the preparation of the composition, as it is preferred that the liquids selected for plasticizing the WITCs of the present invention do not substantially evaporate during or after the preparation of the compositions. That having been said, some boiling away may be tolerated if the mixing step at which the non-volatile liquids plasticize the WITC is brief enough to ensure a residual presence as desired, and/or if the non-volatile liquids are added in sufficient excess to compensate for any partial boiling away that may take place.

For similar reasons of being desirably maintained with the WITC to be plasticized therewith, and retained in the nano-elements comprising it, the non-polar liquid should preferably be unable to migrate to the polar carrier phase. Hence, suitable non-volatile liquids are essentially not miscible in such polar carriers (e.g., water), their solubility in the pure polar carrier or in the liquid phase containing it being as previously detailed for the WITC, namely being of 5 wt. % or less, 4 wt. % or less, 3 wt. % or less, 2 wt. % or less, 1 wt. % or less, of 0.5 wt. % or less, or of 0.1wt. % or less by weight of the carrier or the phase containing it.

Such non-volatile liquids need to be compatible with the WITC of the composition (i.e., able to plasticize it: e.g., decreasing its Tm, Ts or Tg, and/or decreasing its viscosity). A non-volatile liquid adapted for a particular WITC can be selected accordingly by routine experimentation. For instance, given a particular WITC, various non-volatile liquids can be mixed with it, at one or more relative concentrations, and optionally at an elevated temperature, facilitating the plasticizing, and their effects on the WITC being plasticized monitored by thermo-rheology (for their ability to decrease viscosity as a function of temperature) and by thermal analysis (e.g., by DSC, for their ability to decrease the Tm, Ts or Tg of the native WITC). The non-volatile liquids most potent with respect to the particular WITC can be selected accordingly.

Fundamentally, a material or a chemical composition is compatible with another if it does not prevent its activity or does not reduce it to an extent that would significantly affect the intended purpose. Such compatibility may be from a chemical standpoint, for instance, sharing similar functional chemical groups or each material having respective moieties that may desirably interact with one another. This kind of compatibility can be demonstrated by the combined materials forming a homogeneous mixture, rather than separate into different phases. A material would be incompatible with another if it contributes to the degradation of the other material. For illustration, a polar liquid phase would be incompatible with the nano-elements if dissolving them, destabilizing them, improperly charging them to have a charge incompatible with their intended use, and so on.

Materials should also be compatible with the methods used for the preparation of the composition, not being adversely affected by any of the steps the material would be subjected to in the process, nor being volatile (or otherwise eliminated) at the temperature(s) they are incorporated in the compositions. Understandingly, the materials need also be compatible with their intended use, which in the present case may include for illustration being biocompatible, non-irritating, non-immunogenic, and having any such characteristic providing for their regulatory approval at a concentration adapted for efficacious pharmaceutical and/or agrochemical compositions to be used as herein-disclosed.

While compatibility is required between the WITC and the non-volatile liquid, dissolution of the WITC within the non-volatile liquid is unwanted, and as such, the non-volatile liquid does not function as a solvent with respect to the WITC, but as a plasticizing agent intended to remain therewith.

It is believed that a solvent, generally being made of relatively small molecules, is able to enter between molecules of WITC and distance them one from the other, to such an extent that the WITC readily dissolves within the solvent, forming a homogeneous solution even at room temperature. As used herein, the term “solvent” refers to a liquid wherein more than about 5 wt. % of the WITC can dissolve at room temperature.

A non-volatile (e.g., plasticizing) liquid, in contrast, being typically larger in size compared to molecules of solvents, would not readily separate WITC molecules to form a solution at room temperature, at which it could at best provide for a preliminary swelling. Elevated temperatures are required to move the WITC molecules sufficiently away from one another to allow enough non-volatile liquid to enter within the formed gaps. Even under such favorable conditions, mixing may be required to homogeneously form the plasticized WITC. Such elevated temperatures can be equal to or higher than at least one of the first Tm, Ts, and Tg of the WITC.

Non-volatile liquids suitable for the present invention can be selected from: monofunctional or polyfunctional aliphatic esters (such as dimethyl glutarate, dimethyl maleate, dimethyl methyl glutarate, dipropylene glycol dibenzoate and lactic acid isoamyl ester); fatty esters (such as 2-ethylhexyl lactate, benzyl benzoate, butyl butyryl lactate, C₁₂-C₁₅ alkyl benzoate, a mixture of caprylyl caprate and caprylyl caprylate, decyl oleate, dibutyl adipate, dicaprylyl carbonate, dibutyl maleate, dibutyl sebacate, diethyl succinate, ethyl oleate, glyceryl monooleate, glyceryl monocaprate, glyceryl tricaprylate, glyceryl trioctanoate, isopropyl myristate, isopropyl palmitate, L-menthyl lactate, lauryl lactate, n-pentyl benzoate, PEG-6 caprylic/capric glycerides, propylene glycol monolaurate, propylene glycol mono-caprylate, triacetin, triethyl citrate, triethyl o-acetylcitrate, tris(2-ethylhexyl) o-acetyl-citrate, tributyl o-acetylcitrate and tributyl citrate); cyclic organic esters (such as decanoic lactone, gamma decalactone, menthalactone and undecanoic lactone); aromatic esters (such as diethyl phthalate); terpenes (such as citronellol, eugenol, farnesol, hinokitiol, linalool, menthol, menthone, neridol, terpineol and thymol); aromatic alcohols (such as benzyl alcohol); aromatic ethers (such as phenoxy ethanol); aldehydes (such as cinnamaldehyde); and combinations thereof.

In some embodiments, the non-volatile liquid that may be used to plasticize a WITC as herein-disclosed is a polyfunctional aliphatic ester (PFAE), being a diester derivative of common dicarboxylic acids: namely adipic (C₆), azelaic (C₉) and sebacic (C₁₀) acids, the alcohol portion of the diesters generally falling in the C₃-C₂₀ carbon number range, including linear and branched, even and odd numbered alcohols. In particular embodiments, the non-volatile liquid, being a PFAE, is selected from dibutyl adipate (e.g., commercially available as Cetiol® B), dibutyl sebacate, triethyl O-acetylcitrate (e.g., commercially available as Citrofol® AII), C₁₂-C₁₅ alkyl benzoate (e.g., commercially available as Pelemol® 256) and dicaprylyl carbonate (e.g., commercially available as Cetiol® CC). In another particular embodiment, the non-volatile liquid is the aromatic ester, benzyl benzoate.

When the WITC is non-biodegradable, additional non-volatile liquids can be used. Such additional liquids can be mineral oils, natural oils, vegetal oils, essential oils, synthetic oils and combinations thereof, provided they preferably fulfill present requirements.

### Polar Medium

The liquid medium forming the continuous phase in which the nano-elements including the WITC(s) and the carrier-insoluble active agent are dispersible is polar. In some embodiments, the liquid phase consists essentially of a polar carrier, whereas in other cases additional components can be present within the polar carrier. Such additional components can be, for illustration, surfactants, carrier-soluble active agents, or any other additives conventionally present in pharmaceutical and/or agrochemical compositions. A polar carrier suitable for the present invention can be selected from a group comprising water, glycols (e.g., propylene glycol, dipropylene glycol, and 1,2-butanediol 1,3-butanediol, 1,4-butanediol, 2-ethyl-1,3-hexanediol and 2-methyl-2-propyl-1,3-propanediol), glycerols including glycerol, precursors and derivatives thereof (e.g., acrolein, dihydroxyacetone, glyceric acid, tartronic acid, epichlorohydrin, glycerol tertiary butyl ether, polyglycerol, glycerol ester and glycerol carbonate), formamide, acetonitrile, and combinations thereof.

A polar medium may be formed of one or more suitable polar carriers, the resulting liquid being often referred to as an aqueous solution (or an aqueous phase) when water is the preponderant polar carrier. In some cases, a liquid deemed not sufficiently polar by itself (such as a fatty alcohol) can be present in the liquid phase in addition to the polar carrier(s), provided that the liquid insufficiently polar to form the entire liquid polar phase is a) soluble in the main polar carrier (e.g., having a water-solubility of 5 wt. % or more) so as to form a unique liquid phase therewith; and b) the overall polarity of the liquid phase is maintained. The polarity index of the resulting liquid phase may be of 3 or more, 4 or more, or 5 or more, water having for reference a polarity index of 9-10.

As the polarity index of a solvent refers to its relative ability to dissolve in test solutes, a liquid may additionally or alternatively be classified as polar or non-polar in view of its dielectric constant (Er). Liquids having a dielectric constant of less than 15 are generally considered non-polar, while liquids having a higher dielectric constant are considered polar, the relative polarity of a liquid increasing with the value of the dielectric constant. Preferably, the polar carrier suitable for the present compositions has a dielectric constant of 20 or more, 30 or more, 40 or more, 50 or more, or 60 or more, as established at room temperature. For illustration, the dielectric constant of propylene glycol is 32, the dielectric constant of glycerol is 46, and the dielectric constant of water is 80. While for simplicity, this guidance is provided for a neat polar carrier, this in fact should preferably apply to the entire polar liquid phase prepared therefrom (e.g., including additional polar-soluble materials and/or consisting of a mixture of liquid carriers). Noticeably, a liquid polar phase can be constituted of a mix of formally polar solvents (e.g., having ϵᵣ≥15) with formally non-polar ones (e.g., having ϵᵣ<15), as long as their respective volume allows for the entire liquid phase to be polar (e.g., having ϵᵣ≥15). The dielectric constant of a liquid is typically provided by the manufacturer but can be independently determined by any suitable method, such as described in ASTM-D924.

As discussed, the composition of the polar liquid phase should be such that the nano-elements including the WITCs (and optional carrier-insoluble active agents and/or SFAs) can remain essentially non-soluble and stably dispersed therein, with no significant leaching of the contents of the nano-elements into their surrounding medium, when undesired. When the nano-elements are core-shell nano-elements, the polar liquid phase should furthermore enable a suitable charging thereof, as adapted, for instance, for a desired distribution of their delivery and efficacy on or across barriers.

As the polar medium may comprise additional liquids and/or materials dissolved therein, the polar carrier can constitute at least 50 wt. %, at least 60 wt. %, at least 70 wt. %, at least 80 wt. %, or at least 90 wt. %, by weight of the liquid phase.

In a particular embodiment, the polar carrier comprises water (e.g., 45 wt. % water, 45wt. % propylene glycol, and 10 wt. % of a fatty alcohol), consists of water (e.g., including between 51 wt. % and 80 wt. % of water), consists essentially of water (e.g., including between 81 wt. % and 99 wt. % of water), or is water.

As mentioned, the principles above detailed with respect to the polar carrier in which the nano-elements can be prepared may similarly apply to any other liquid vehicle to which the nano-elements might be transferred for the preparation of a liquid dosage form of a pharmaceutical and/or agrochemical composition as suited for their elected route of administration.

### Surfactants

Some nano-elements may remain nano-dispersed in a liquid medium in view of their inherent chemical properties, the nano-elements (the cores or the shells, if present) for instance having a charge sufficient to ensure repulsion of the particles, consequently ensuring their stable dispersion.

In such cases, and regardless of the underlying rationale, the nano-elements can be considered self-emulsifying (e.g., remaining as discrete individual nano-elements in the liquid medium in absence of a surfactant devoted to that purpose). The nano-elements are deemed dispersed, when the particles have reached a desirable size (and/or particle size distribution) and “stably dispersed” when the particles can retain a desirable size (or PSD) over time. The original size and PSD can be as determined upon completion of preparation of the nano-particles, or at any other desired timepoint. As size measurements may vary between repeats at a same time point, a size or PSD at a subsequent time point is considered substantially similar to values obtained at a previous time point, if being within 10% or within 5% of previously determined values. Therefore, the particles are considered stably dispersed if measurements of their sizes and/or size distribution do not vary by more than 10% or 5% for at least 1 day, for at least 2 days, or for at least 3 days. Such stability can be assessed under any desired storage conditions, either at room temperature, at cooler temperatures (e.g., 4-8° C.), or on the contrary at higher temperatures (e.g., 30-40° C.), when accelerated stability tests are desired.

While the compositions used in the present invention can be stable as a result of the constituents of the core or core-shell nano-elements and/or their environmental conditions (e.g., pH of liquid medium), in some embodiments, the compositions may further comprise at least one (dedicated) surfactant, for the nano-elements to remain stably dispersed (hence also in their intended size range).

Depending on their chemical formula, the surfactants can either be miscible with the WITCs (and any other components included in the core) or soluble/dispersible in the polar carrier wherein the nano-suspension is formed.

Surfactants can be anionic, cationic, amphoteric or non-ionic surfactants. If the surfactants are non-ionic or amphoteric with an overall near neutral charge, and moreover insoluble in the polar carrier and miscible with the WITCs, they can be included in the core of the nano-elements, their migration towards an outer surface of the cores not significantly affecting the charge or chargeability of the nano-elements (as opposed to SFAs intended for this purpose). Such surfactants, that for simplicity can be referred to as “charge neutral surfactants”, may promote interactions with agents found in the liquid phase by dipole: dipole forces, or any other mechanism wherein the charging of the surfactant is not necessary. For illustration, a carrier-insoluble non-ionic surfactant present in the nano-elements may form through its outward end dipole: dipole interactions with carrier-soluble anionic surfactants found in the polar liquid phase.

If the surfactants are relatively soluble in the polar carrier, or for simplicity of preparation regardless of solubility, they can be added to the liquid phase regardless of the charge they may, or not, form therein.

Anionic surfactants that can be added to/present in the polar carrier can be selected from the group including: alkyl sulfates (e.g., sodium lauryl sulfate, ammonium lauryl sulfate and ammonium laureth sulfate); sulfosuccinates (e.g., disodium lauryl sulfosuccinate, disodium laureth sulfosuccinate, sodium dioctyl sulfosuccinate and their mixtures with sulfonic acids and lauramidopropyl betaine); alkyl benzene sulfonates (e.g., salts of toluene sulfonic acid, xylene sulfonic acid, cumene sulfonic acid (e.g., sodium, potassium, calcium, ammonium salts)); acyl methyl taurates (e.g., sodium methyl lauroyl taurate and sodium methyl cocoyl taurate); acyl sarcocinates (e.g., sodium lauroyl sarcosinate, sodium cocoyl sarcosinate and sodium myristoyl sarcosinate); isethionates (e.g., sodium butyl isethionate, sodium capryloyl isethionate and sodium lauroyl isethionate); propyl peptide condensates; monoglyceride sulfates; ether sulfonates and fatty acid salts (e.g., sodium stearoyl lactylate). Alkyl benzene sulfonate salts, generally having a larger hydrocarbon residue (between 20 and 30 carbon atoms) and hence being carrier-insoluble, may also serve as SFAs when mixed with the WITCs.

Cationic surfactants that can be added to/present in the polar carrier can be selected from the group including: quaternary ammonium compounds, most being highly soluble in water (e.g., benzalkonium chloride and trimethyl ammonium methyl sulfate). Quaternary ammonium compounds being relatively water-insoluble fatty amines (e.g., stearalkonium chloride and cetrimonium chloride) may also be utilized as SFAs when mixed with the WITCs.

Amphoteric surfactants that can be added to/present in the polar carrier, or present in the nano-elements if miscible with the WITCs, can be selected from the group including: betaines (e.g., cocamidopropyl betaine); alkylamphopropionates (e.g., cocoamphopropionate); alkylimino-propionates (e.g., sodium lauraminopropionate); and alkylamphoacetates (e.g., cocoampho-carboxyglycinate.

Non-ionic surfactants that can be added to/present in the polar carrier, or present in the nano-elements if miscible with the WITCs, can be selected from the group including: fatty alcohols (e.g., cetearyl alcohol); ethoxylated fatty alcohols (e.g., C₈-C₁₈ alcohol polyglycol, polyoxyl 6 stearate and polyoxyl 32 stearate); poly(ethylene glycol) block copolymers (e.g.; poloxamer); ethylene oxide (EO)/propylene oxide (PO) copolymers; alkylphenol ethoxylates (e.g., octylphenol polyglycol ether and nonylphenol polyglycol ether); alkyl glucosides and polyglucosides (e.g., lauryl glucoside); fatty alkanolamides (e.g., lauramide diethanolamine and cocamide diethanolamine); ethoxylated alkanolamides; ethoxylated fatty acids; sorbitan derivatives (e.g., polysorbates, sorbitan laurate, sorbitol, 1,4-sorbitan, iso-sorbide and 1,4-sorbitan triester, PEG-80); alkyl carbohydrate esters (e.g., saccharose fatty acid monoester); amine oxides; ceteareths; oleths; alkyl amines (other than fatty amines); fatty acid esters (e.g., ascorbyl palmitate, ethylene glycol stearate, polyglyceryl-6 esters, polyglyceryl-6 pentaoleate, polyglyceryl-10 pentaoleate and polyglyceryl-10 pentaisostearate); polyoxylglycerides (e.g., oleoyl polyoxyl-6 glycerides); natural oil derivatives; ester carboxylate (e.g., D-α-tocopherol polyethylene glycol succinate (vitamin E TPGS)); and urea.

These surfactants may be categorized into emulsifiers and hydrotropes, according to their mechanism of action. Emulsifiers typically include a relatively large hydrophobic or hydrophilic part and can readily form micelles (thus being characterized by a critical micelle concentration (CMC) value). As a rule, emulsifiers typically relate to surfactants ensuring the dispersion of one liquid into another, the liquids having opposite polarity, whereas dispersants relate to surfactants ensuring the dispersion of a solid into a liquid. As the present method may provide for nano-emulsions and nano-dispersions, surfactants referred to as emulsifiers at a step in which the nano-suspension is an emulsion (e.g., at an elevated temperature), may in fact become dispersants, to the extent that an initial nano-emulsion later yields a nano-dispersion at a lower temperature. Hence, as used herein, the term “emulsifier(s)” also includes surfactants otherwise known as dispersants.

Emulsifiers that are lipophilic in nature, i.e., include a relatively large hydrophobic part, are in principle more suitable to be combined with the WITCs (and any other material not miscible in the polar carrier, e.g., a non-volatile liquid, an active agent, or an SFA), though they may alternatively be added to the liquid phase, their incorporation in each phase depending in practice on their chargeability, or lack thereof, as previously explained. They may be referred to as polar-carrier-insoluble emulsifiers (or surfactants, in general). These relatively hydrophobic emulsifiers generally have HLB values of 9 or less, 8 or less, 7 or less, or 6 or less, on Griffin scale.

Emulsifiers that are more hydrophilic in nature have a relatively large hydrophilic part and would only be compatible with the polar phase of the composition. They may be referred to as polar-carrier-soluble emulsifiers (or surfactants, in general). Such relatively hydrophilic emulsifiers generally have HLB values of 11 or more, 13 or more, 15 or more, 17 or more, or 20 or more.

Emulsifiers having HLB values within the range of 9 and 11 are considered “intermediate”, the hydrophobic and hydrophilic parts of such emulsifiers being fairly well-balanced. Such intermediate emulsifiers can be added in the present methods either to the WITCs (provided they cannot charge the cores significantly) or to the polar carrier and may accordingly be found in the nano-elements or in their medium.

Emulsifiers can be selected from a group comprising alkyl sulfates, sulfosuccinates, C₇-C₁₀ alkyl benzene sulfonates, acyl methyl taurates, acyl sarcocinates, isethionates, propyl peptide condensates, monoglyceride sulfates, ether sulfonates, ester carboxylates, betaines, alkylampho-propionates, alkyliminopropionates, alkylamphoacetates, fatty alcohols, ethoxylated fatty alcohols, poly(ethylene glycol) block copolymers; ethylene oxide (EO)/propylene oxide (PO) copolymers, alkylphenol ethoxylates, alkyl glucosides and polyglucosides, fatty alkanolamides, ethoxylated alkanolamides, ethoxylated fatty acids, sorbitan derivatives, alkyl carbohydrate esters, amine oxides, ceteareths, oleths, alkyl amines, fatty esters, polyoxylglycerides, natural oil derivatives and ester carboxylate.

In particular embodiments, the emulsifier is selected from: vitamin E TPGS, poly (ethylene glycol) block copolymer, a mixture of polyoxyl 6 stearate type I, ethylene glycol stearates and polyoxyl 32 stearate type I (such as commercially available as Tefose® 63 from Gattefossé, France), mixtures comprising olive oil-derived extracts (such as commercially available under the brand Olivatis® from Medolla Iberia, Spain), ascorbyl palmitate, polyglyceryl-10 pentaoleate, polyglyceryl-10 pentaisostearate, oleoyl polyoxyl-6 glycerides (such as commercially available as Labrafil® M 1944 CS from Gattefossé, France), disodium laureth sulfosuccinate, disodium lauryl sulfosuccinate, a mixture of disodium lauryl sulfosuccinate, sodium C₁₄-C₁₆ Olefin sulfonate and lauramidopropyl betaine (such as commercially available as Cola®Det EQ-154 from Colonial Chemical, USA) and a mixture of olive oil and glutamic acid (such as commercially available as Olivoil® glutamate from Kalichem, Italy).

While surfactants acting as emulsifiers are generally sufficient to stabilize nano-elements of the present compositions that are not self-emulsifiable, the Inventors have found that when WITCs are present at a relatively high concentration, as enabled by the present teachings, the addition of another type of surfactants, namely hydrotropes, assisted in achieving a satisfactory stability.

Hydrotropes, contrary to emulsifiers, contain a relatively shorter lipophilic chain. As the lipophilic portion of the hydrotropes is generally too short to allow micelle formation, the hydrotropes alternatively solubilize hydrophobic compounds in the polar carrier and permit co-emulsification, together with the emulsifier. Generally, hydrotropes are miscible mainly in the polar carrier phase (e.g., aqueous phase) of the nano-suspension, and are characterized by having HLB values of 10 or more, 12 or more, 15 or more or 18 or more.

Suitable hydrotropes can be selected from the group including: sodium dioctyl sulfosuccinate, urea, adenosine triphosphate and salts (e.g., sodium, potassium, calcium, ammonium) of toluene sulfonic acid, xylene sulfonic acid and cumene sulfonic acid.

In a particular embodiment, the hydrotrope is selected from: sodium dioctyl sulfosuccinate, urea and a salt of xylene sulfonic acid, such as ammonium xylenesulfonate.

### Active Agents

The nano-elements having a core made of the WITC further comprise one or more active agents, the nano-elements serving as nano-carriers delivering such active agents as medications.

The active agents may be polar-carrier-insoluble active agent(s), being generally miscible with the WITCs and any components included within the matrix of the nano-elements' cores, e.g., with the non-volatile liquid and the hydrophobic tails of the SFAs entrapped within the cores, when such materials are present. The constituents of the nano-elements are miscible one with the other when forming a unique phase, this property yielding in turn cores being made of a continuous matrix. The carrier-insoluble (and WITC-miscible) active agents may be entirely contained (if substantially apolar) or partially entrapped within the core (if amphiphilic).

Similarly to the WITC, SFA and non-volatile liquid described above, the WITC-miscible active agent should have a solubility of 5 wt. % or less, 4 wt. % or less, 3 wt. % or less, 2 wt. % or less, 1 wt. % or less, 0.5 wt. % or less, or 0.1 wt. % or less by weight of the polar carrier or the liquid phase including it, to be considered polar-carrier-insoluble.

Alternatively, or additionally, the active agents may conversely be polar-carrier-soluble active agent(s), in which case they are typically disposed in shells surrounding the core, the shell being in one embodiment a second shell indirectly surrounding the core and anchored thereto via a first shell formed of amphiphilic molecules (e.g., other active agents or SFAs). The second shell may be composed of more than one layer of a carrier-soluble active agent(s). Excess of polar-carrier-soluble active agent(s) may additionally be found in dissolved form within the polar carrier.

In some embodiments, the active agent, regardless of where it is disposed in the nano-elements, has a molecular weight of no more than 1,000 g/mol, or no more than 500 g/mol, in particular if passive cell permeability is expected at the site of delivery. However, as an active agent may be effective for the diagnosis or treatment of a condition without having to penetrate particular target cells, the active agents that can be incorporated into the cores or shells of the nano-elements need not be limited to such MW.

Thus, advantageously, the active agent, whether apolar, amphiphilic or polar, can also have a MW of more than 1,000 g/mol, being for instance of at least 1,200 g/mol, at least 1,400 g/mol, at least 1,600 g/mol, or at least 1,800 g/mol. Typically, the MW of an active agent that can be incorporated into the present nano-elements (e.g., having an average diameter of 1,000 nm or less, or even 200 nm or less) does not exceed 500 kDa. In some embodiments, the MW of the active agent is 400 kDa or less, 300 kDa or less, 200 kDa or less, or 100 kDa or less. In particular embodiments, the active agent has a molecular weight not exceeding 50 kDa, not exceeding 40kDa, not exceeding 30 kDa, not exceeding 20 kDa, not exceeding 10 kDa, or not exceeding 5 kDa.

While selected according to the diagnostic, prophylactic or therapeutic activity they are to provide to the present medications, active agents may have secondary functions, and for illustration WITC-miscible/polar-carrier-insoluble active agents may optionally act as plasticizers, when capable of decreasing the viscosity of the WITC within the core of the nano-elements, or polar-carrier-soluble active agents may optionally act as surfactants, when capable of stabilizing core-shell nano-elements in the liquid phase they are dispersed in.

While the present invention is mainly concerned by the ability of its nano-elements having a core made of WITCs to deliver active agents contained or entrapped therein, and/or anchored thereto, so as to provide efficacious pharmaceutical and/or agrochemical medications, such drugs need not be the only ones present in a final dosage form. Other active agents could be included in compositions used according to the present teachings. The other drugs can be, for instance, soluble within the liquid dosage form including the present nano-elements or can be added to a suitable dosage form in a drug carrier distinct from the present nano-elements, the additional active agents being external to the nano-elements. For illustration, the composition can be adapted for administration as a liquid dosage form, in which case the additional active agents can be dispersed or dissolved in the liquid vehicle in which the nano-elements are dispersed.

Active agents suitable to be incorporated in the pharmaceutical compositions for the preparation of medication for human or non-human (i.e., veterinary) uses of the present invention, as can be administered by non-dermal routes to a subject in need thereof, can be found e.g., in the FDA's Drugs@FDA database, in the European Medicines Agency (EMA)'s European Public Assessment Reports (EPARs) database, or in any similar authoritative list issued by relevant professionals in the pharmaceutic industry.

Active agents suitable for agrochemical compositions for the preparation of a medication as can be administered to an subject in need thereof by other than exposed surfaces of said object, can be found e.g., in the Pesticides Product Information System (PPIS) maintained by the US Environmental Protection Agency (EPA), in the EU Pesticides Database maintained by the European Commission's Directorate-General for Health and Food Safety (DG SANTE), or in any similar authoritative list issued by relevant professionals in the agrochemical industry.

### Volatile Organic Compounds

Methods for preparing nano-materials are abundantly described in the literature, the main ones suitable for hydrophobic compounds, such as the materials used in the present invention, involving emulsification. The traditional emulsification methods suited for this purpose include oil-in-water (O/W) emulsification or W/O/W emulsification, generally achieved by solvent evaporation or solvent displacement (e.g., performed by solvent diffusion or salting-out). These methods are cumbersome and time-consuming, some to an extent being commercially unfeasible. More importantly, they typically result in solvents remaining residually (but not negligibly) trapped within the nano-particles prepared by such methods, which can be particularly critical when the solvent being used is a volatile organic compound (VOC) having a high vapor pressure and low boiling point at room temperature.

As used herein, the term “volatile organic compound” (“VOC”) refers to an organic compound having a vapor pressure of 0.1 kPa or more, as measured at a temperature of 20° C.

Typically, when nano-particles were reported to have been obtained following dissolution of a polymer in a volatile solvent and its subsequent evaporation, the authors remained understandingly silent about their true VOC contents, in view of the difficulties to fully eliminate. When relatively low amounts of VOCs were actually measured and reported, the particles obtained were at least micro-particles (e.g., having a particle size of 2-100 micrometer (μm)) and not nano-particles in the sub-micron range.

As opposed to methods commonly used for preparing micro-or nano-particles, the preparation of the compositions used in the present invention does not involve an emulsification technique which requires addition of a solvent (such as a VOC) and dissolution of the WITC (specifically WITP) therein, requiring the subsequent removal of the solvent. When volatile solvents are later removed from particles conventionally prepared (presumably by lengthy and burdensome methods), few phenomena may be observed. The elimination of the solvent typically yields porous and friable particles, moreover, as the elimination of the solvent may concurrently require the elimination of the medium in which the particles may be dispersed, this process inherently causes the collapse of the dispersion and the drying of the particles, which may then turn into aggregates or agglomerates. Once dried, in an attempt to eliminate the solvent having served for their preparation, the particles conventionally prepared are believed to have typically a poor dispersibility as individual particles and furthermore to have a poor malleability, if any, their consistency being substantially solid (i.e., having a dramatically high “viscosity”). Such porous particles, prepared according to methods of the art, are believed to have drawbacks overcome by the present non-porous nano-elements.

Thus, VOCs such as conventionally used acetone, acetonitrile, aniline, benzene, carbon tetrachloride, chloroform, cyclohexanone, dichloromethane, dioxane, dimethylesulfoxide, ethyl acetate, hexafluoroisopropyl alcohol, methylene chloride, N,N-dimethylformamide, 2-nitropropane, 1,1,2,2-tetrachloroethane, tetrahydofuran, 1,1,2-trichloroethane and toluene are essentially absent from nano-elements according to the present teachings, either as individual solvents or as blends of two or more VOCs.

In some embodiments, the nano-elements of WITC contain less than 2 wt. %, less than 1.5 wt. %, or preferably less than 1 wt. %, less than 0.5 wt. %, less than 0.4 wt. %, less than 0.3 wt. %, less than 0.2 wt. %, or less than 0.1 wt. % of a VOC or a blend thereof by weight of the nano-elements. In particular embodiments, the nano-elements contain less than 0.09 wt. %, less than 0.08 wt. %, less than 0.07 wt. %, less than 0.06 wt. %, less than 0.05 wt. %, less than 0.04 wt. %, less than 0.03 wt. %, or less than 0.02 wt. % of a VOC or a blend thereof by weight of the nano-elements. In some embodiments, the nano-elements are devoid of any VOC, but may contain up to 0.001 wt. % (which corresponds to 10 parts per million-ppm), up to 0.002 wt. % (20 ppm), up to 0.003 wt. % (30 ppm), up to 0.004 wt. % (40 ppm), up to 0.005 wt. % (50 ppm), up to 0.006 wt. % (60 ppm), up to 0.007 wt. % (70 ppm), up to 0.008 wt. % (80 ppm), up to 0.009 wt. % (90 ppm), or up to 0.01 wt. % (100 ppm) of a VOC or a blend thereof by weight of the nano-elements. The above-mentioned amounts are cumulative in case of more than one VOC being present in the nano-elements.

In particular embodiments, the nano-elements contain between 0 wt. % and 1 wt. %, between 0.0001 wt. % and 0.5 wt. %, between 0.0005 wt. % and 0.5 wt. %, between 0.001 wt. % and 0.5 wt. %, between 0.002 wt. % and 0.4 wt. %, between 0.003 wt. % and 0.3 wt. %, between 0.004 wt. % and 0.2 wt. %, between 0.005 wt. % and 0.2 wt. %, between 0.005 wt. % and 0.1 wt. %, between 0.001 wt. % and 0.09 wt. %, between 0.002 wt. % and 0.08 wt. %, between 0.003 wt. % and 0.07 wt. %, between 0.004 wt. % and 0.06 wt. %, or between 0.005 wt. % and 0.05 wt. % of a VOC or a blend thereof by weight of the nano-elements.

It is stressed that the afore-said low levels of VOCs are provided with respect to the nano-elements, as the liquid phase in which they are dispersed may tolerate higher amounts of such materials, as may be allowed by the resistance of the nano-elements to solubilization and/or as may be authorized by relevant regulatory authorities governing the use of compositions for any of the purposes for which the present compositions are suitable.

The nature and amount of hypothetical VOCs in nano-elements can be determined by routine analysis, e.g., by eliminating the polar carrier, and analyzing the nano-elements isolated therefrom by, e.g., Gas Chromatography (GC) combined with Mass Spectrometry (MS) for quantitative determination. Exemplary standard methods for determining presence of VOCs are described in ASTM D4526 or VDA 277, the analysis being performed at a temperature of 90° C. and the absolute quantities measured by MS. Alternatively, once the presence of a VOC is estimated by GC, a sample can be tested for weight loss at 90° C.

As the methods used for preparing the present nano-carriers do not involve the dissolution of the WITC in a solvent (such as a VOC) and its later removal resulting in porous particles, the core of the present nano-elements is in contrast substantially non-porous.

### Core-Shell Nano-Elements

When it is desired for the nano-elements within the compositions used in the present invention to be charged differently, shell-forming agents (SFAs) may be combined with the WITC(s) and WITC-miscible agent(s), resulting in core-shell nano-elements having a positive or negative charge when placed in a polar carrier, the charge of the core-shell nano-elements being other than the charge of nano-elements only having a similar core.

The added SFAs are not intended to entirely remain within the core of the nano-elements, on the contrary, under the conditions elected for the preparation of the nano-elements, they are believed to migrate towards the cores' outer surface, allowing their hydrophilic heads to be exposed to the polar carrier surrounding the nano-elements, while their hydrophobic tails tend to remain within the water-insoluble environment of the core. This phenomenon results in the formation of a shell chargeable in the presence of water, which can be confirmed by the change in charge of the nano-elements with or without the SFAs.

As mentioned above, WITCs are generally negatively charged, and accordingly, a core comprising a WITC (and other WITC-miscible ingredients) is expected to display a negative charge in an aqueous polar liquid (e.g., in water). However, when SFAs are further included within the composition, a shell of the SFA hydrophilic heads is formed. When positively-charged in aqueous environment, such shell is masking the core, reducing its negative charge, and may even provide a positive charge to the core-shell nano-elements depending on the respective charge density, on the amounts of the core and shell materials and on the liquid environment (e.g., pH) which may modulate said charges. Negatively-charged SFA may provide a more negative charge to the core-shell nano-elements.

Noticeably, the SFAs forming the shell of the nano-elements are not covalently bound to the WITC of the core, which is believed to advantageously maintain the original activity of each of the molecules of the core and the shell, none of their moieties being involved in covalent binding through electron sharing that could have reduced or modified their respective contribution to the potency of the composition. Moreover, the fact that the SFAs are not covalently bound to the WITCs allow them to migrate to the outer surface of the nano-elements to form a shell thereon under suitable conditions.

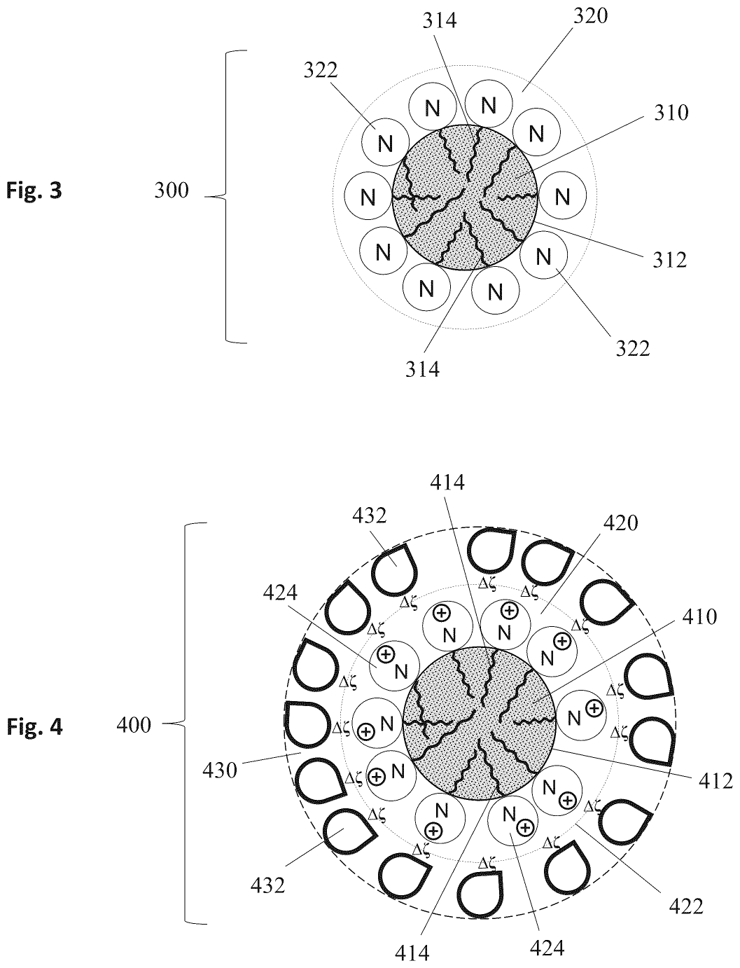

FIG. 3 schematically depicts a core-shell nano-particle 300 in a non-aqueous polar carrier. The nano-element contains a core 310, composed of a WITC (depicted as the grey background) and a WITC-miscible active agent (depicted as small black dots), and any other optional ingredient miscible with the WITC and insoluble in the polar carrier, such as a non-volatile liquid, the core entrapping at least part of the hydrophobic tails 314 of a SFA. The polar heads 322 of the SFA (being illustrated by a fatty amine in the figure, depicted by circled “N”) are pointing away from the core's surface 312, forming the nano-element's shell 320. The absence of water prevents the charging of the hydrophilic heads of the SFAs.

The electrostatic-charging of the nano-elements, especially as core-shell nano-elements but not exclusively, facilitates their further coating by an external shell of carrier-soluble active agents suitably having an opposite charge. The resulting core-multi-shells form more moderately charged or even uncharged nano-elements. The moderately charged core-multi-shells can typically maintain their original charge (i.e., positive or negative), but less than their respective core-shell, lacking the second shell of the active agent. Alternatively, the core-multi-shells can become oppositely charged, e.g., a positively-charged core-shell nano-element can become a negatively-charged core-multi-shells nano-element, provided that collapsing of the composition (e.g., by aggregation of the nano-elements at their isoelectric point) can be avoided.

FIG. 4 schematically depicts such a core-multi-shells nano-particle 400, coated with a carrier-soluble active agent, in an aqueous environment.

Similar to the nano-particle 300 previously described in FIG. 3, nano-particle 400 is composed of a core 410, in which at least part of the hydrophobic tails 414 of the SFAs are entrapped. Unlike nano-particle 300, which is dispersed in a non-aqueous polar carrier, the aqueous environment, in which nano-particle 400 is dispersed, allows for the polar heads 424 of the SFAs (positively-chargeable fatty amines in the figure, for illustration) to be charged, forming a now positively charged nano-particle first shell 420, surrounding the core's surface 412. This charging of the first shell allows the anchoring of carrier-soluble active agent molecules 432 to the surface of the nano-particle first shell 422, resulting in the formation of a second shell 430 comprising the active agent, having a zeta potential differential (Δζ) between the surface zeta potential of core-shell nano-elements and the surface zeta potential of the carrier-soluble active agent. The polar-carrier-soluble active agent molecules may be negatively charged, resulting in a relatively larger Δζ between the first shell 420 of the nano-particle, being positively charged, and the active agent molecules 432. Alternatively, the active agent molecules may be positively charged in the polar carrier, resulting in a relatively smaller Δζ, and a relatively weaker attraction compared to negatively charged molecules. Yet, as long as the Δζ is of 5 mV or more, the conditions are sufficient for the formation of the second shell 430.

While FIG. 4 depicts an exemplary first shell surrounding the cores enabling their positive charging, so that the carrier-soluble active agents adapted to form a second shell thereon can be negatively charged, the core-shell nano-elements can alternatively be negatively charged, so as to permit the formation of second shells made from positively charged carrier-soluble active agents. Negatively charged inner core-shell can be obtained by modulating the environment of the nano-element, or by using for the formation of the first shell, a fatty compound having a negatively chargeable hydrophilic head such as fatty acids.

In particular embodiments, the core-shell nano-elements are positively charged and the carrier-soluble active agent molecules are negatively charged in the liquid environment serving for the anchoring of such active agents to the core of the nano-elements via a first shell including the hydrophilic heads of the SFAs.

Without wishing to be bound by theory, it is believed that the charge of the nano-elements having at first only a single shell is mainly contributed by the hydrophilic heads of the SFAs, which become charged when exposed to an aqueous environment, depending on the pH in the polar carrier and nature of the SFA.

For instance, when the SFA is a fatty amine, the amine groups in the first shell become protonated at an acidic suitable pH, the protons being donated from the water. Hence, when the polar carrier is an aqueous polar carrier (e.g., water, or an aqueous mixture), the nano-elements can develop a positive charge. Alternatively, when the SFA is a fatty acid, the acidic groups in the first shell can become negatively charged (e.g., at a basic pH) due to de-protonation, and the formation of a carboxylate.

However, when a non-aqueous polar carrier is used during the preparation of the nano-elements, a coordinate bond is believed to form with the SFA hydrophilic heads of the first shell, a polar carrier unable to protonate the molecules of the first shell “masking” any positive charge that may have otherwise potentially developed on the surface of the core-shell nano-elements in presence of a liquid enabling protonation. In such case, or when the charge detected in the liquid carrier is deemed insufficient, un-masking of the charge or its increase may be performed by replacing at least part of the polar carrier by water (such as, by combining the nano-suspension with water), thus creating an aqueous environment wherein the charge can be made available or develop.

As known to persons skilled in the measurement of charge and zeta potential of compositions, it is stressed that in this particular context an aqueous polar carrier need only to contain an amount of water (e.g., ≥5 wt. %) sufficient to promote the formation of positive or negative charge of the SFA within the shell (e.g., protonation of the fatty amines or de-protonation of the fatty acids). Such values, which can be measured with any suitable equipment, can be determined in the composition “as is” or in a diluted sample thereof.

While the water itself is capable of creating an environment that promotes charging of the nano-elements, a pH-modifying agent, may be added to further promote the charge formation. For instance, when the SFA is a fatty amine, an acidic agent is believed to react with the polar carrier molecules (supposedly by hydrogen bonding), distancing them from the shell surface of the nano-particles, and leaving the amine heads exposed to the proton-rich acidic environment, thus allowing the formation of a positive charge on the shell surface. Alternatively, a base may be added to the polar carrier, contributing to the positive charging of the amines, even if slightly less than an acidic agent. A basic pH-modifying agent may optionally further contribute to the negative charging of a carrier-soluble active agent, if added to the composition. Such addition of an acid or a base to the nano-suspension may be referred to as “acid doping” or “base doping”, respectively.

The charge of materials due to form a second shell, whether or not enhanced by doping, is expected to promote their attraction to the charged core-shell nano-elements, facilitating the formation of core-multi-shells nano-elements via non-covalent electrostatic attraction to the opposite charges. Alternatively, or additionally, the active agents of the second shell may be selected to covalently bind to materials of the first shell (e.g., SFAs, surfactants, etc.).

The amount of the pH-modifying agent added to the composition should be controlled, so as to create an environment that keeps the balance between a sufficient charging of the SFAs, and a sufficient charging of the carrier-soluble active agent, their respective opposite charges being adequate for attraction of one to the other. For instance, the amount of an acidic agent should be sufficiently high to allow optimal positive charging of the amine heads, but sufficiently low so as to keep the carrier-soluble adequately negatively charged.

In some embodiments, the pH-modifying agent is added in an amount resulting in the composition having a pH between 1 and 11, between 1 and 10, between 1 and 9, between 1 and 8, between 2 and 7, or between 2 and 5. In particular embodiments, when wishing to positively charge the core-shell nano-elements, the pH modifying agent is added in an amount which provides for a pH of 7 and less, 6 or less, 5 or less, or 4 or less. In other embodiments, when wishing to negatively charge the core-shell nano-elements, the pH-modifying agent is added to yield a pH of more than 7, more than 8, more than 9, or more than 10.

While pH-modifying agents are typically added to the liquid carrier in which the nano-elements are dispersed, they may alternatively, or additionally, be added to the nano-elements, for instance incorporated into their core, regardless of the number of shells that may surround them. pH-modifying agents suitable for incorporation into the core, from which they would desirably leach out in due time, should be miscible and compatible therewith, and also with the polar carrier.

In some embodiments, the core-shell nano-elements have a positive charge of +5 mV or more, +10 mV or more, +20 mV or more, +30 mV or more, or +40 mV or more, when placed in an aqueous environment at room temperature, the positive charge typically not exceeding +100 mV.

In other embodiments, the core-shell nano-elements have a negative charge between -100 mV and −5 mV, between −80 mV and −5 mV, between −60 mV and −5 mV, between −50 mV and −10 mV, between −40 mV and −20 mV, between −50 mV and −30 mV, or between −60 mV and −30 mV, when placed in an aqueous environment at room temperature.

In further embodiments, the core-shell nano-elements have a near neutral charge between −5 mV and +5 mV, when placed at room temperature in an aqueous environment, for instance, in presence of a non-ionic surfactant.

As the size of particles decreases, their specific surface area increases, hence the amount of materials that may be present on their outer surfaces. As the nano-elements used in the present invention are inter alia characterized by a D_{N}50 of 1,000 nm or less, and particularly 200 nm or less, in some embodiments, the nano-elements can be sufficiently small to reach a specific surface area that is high enough to allow the presence of enough hydrophilic heads in the shell being able to contribute to a desirable positive charge of the nano-dispersion. In such a case, the use of a pH-modifying agent, which may be used to further boost a charge, can be superfluous (as demonstrated in Example 4-II below).

The amount of the carrier-soluble active agent to be added to the core-shell nano-elements may depend inter alia on the particle size of the nano-elements and the surface area accordingly available to attachment for anchoring of external active agents to the core of the nano-elements. Moreover, the respective zeta potentials of the core-shell nano-elements and of the carrier-soluble active agent may also indicate the respective proportions at which they can be present in the composition to optimize the formation of the core-multi-shells nano-elements, avoiding if desired the presence of excess active-agents in the polar carrier. While such excess is permitted, in particular as long as promoting a stabilization of the second shell, too much of unbound active agents can be superfluous if the molecules of the carrier-soluble active agents have a high molecular weight precluding their cellular delivery, if desired.

The attachment of the carrier-soluble active agent to the core-shell nano-elements is enabled or promoted as a result of a difference in zeta potential between the zeta potential (ζ1) of original core-shell nano-elements, induced by the fatty compounds of the first shell, and the zeta potential of the active agent (ζ2), due to form the second shell. In some embodiments, the absolute value of the zeta potential differential (Δζ) defined as Δζ=|ζ2−ζ1| is at least 5 mV, at least 10 mV, at least 15 mV, at least 20 mV, at least 25 mV, or at least 30 mV, as measurable in the presence of water or an aqueous polar carrier. Such values can be determined as described for the charge of the core-shell nano-elements, the conditions (e.g., equipment, temperature and pH) selected for the measurements being the same for the nano-elements serving as anchor and the carrier-soluble active agent intended to form a further shell.

While in preceding paragraphs, the chargeability of the nano-elements has been considered as a mean to form different shells around the cores in liquid environments selected and adapted to favor a respective charging of the components and the formation of core-shell or core-multi-shells, this may not be the sole reason one would want to control the charge of nano-elements. Additional considerations can be the chargeability of the nano-elements, with or without shells surrounding the cores, in the physiological environments to which the medications are to be administered. Considering animals, and human beings for illustration, these creatures are primarily composed of water, aqueous body fluids often constituting most of the body weight.

Under normal physiological conditions, the outer surfaces or walls of internal cells, tissues or organs (such as the blood brain barrier, the walls of blood vessels, cell membranes, etc.) are generally negatively charged. Therefore, the overall charge of the nano-elements in physiological fluids contributed inter alia by the WITCs of the cores, the active agents and, when present, the SFAs may modulate the electrostatic interaction of the nano-elements with charged surfaces and their retention thereon, in turn facilitating delivery of the active agents at such sites, and/or the penetration of the nano-elements through these barriers. It is believed that nano-elements more positively charged (regardless of the number of shells), while expected to be better retained and adhered to the surface of such barriers having an opposite charge, would have a slower passage rate than less positively charged or even negatively charged nano-elements. Negatively charged nano-elements may experience a slight repulsion when transiting via the barrier, but this phenomenon may push them further “down” across the barrier.

Based on this theory, it is believed that the relative penetration of the nano-elements across organ barriers can to some extent be controlled, not only by their dimensions and/or malleability (e.g., viscosity), but also according to their charge (e.g., by the magnitude of their positive or negative charge). Accordingly, the compositions comprising the nano-elements, may be designed for site-specific drug delivery depending on the charge of the nano-elements and the charge of the tissue being targeted under the relevant physiological or pathological conditions.

For illustration, taking into account pharmaceutical products, e.g., intended for treating endothelial dysfunction of endothelial cells lining the inner part of blood vessels. Positively-charged nano-elements, containing suitable active agents (in the core and/or any of shells of the nano-elements) are expected to electrostatically migrate towards the damaged endothelial cells (generally negatively charged), and accumulate on their surface, releasing the active agent they carry. Similarly, negatively-charged nano-elements may be desired in order to penetrate through the negatively-charged blood vessel walls more easily, thus delivering the active agents outside the vascular system when needed.

### Shell-Forming Agents (SFAs)

SFA molecules are miscible in the WITCs forming the core and as such contain a hydrophobic part (aka, a fatty “tail”), being straight, branched, or cyclic, saturated or unsaturated, aliphatic or aromatic, alkyl or aryl chain, the SFAs furthermore including a chargeable hydrophilic part (e.g., an amine, acid or sulfonate “head”) capable of forming a shell surrounding the core.

Typically, the SFAs are liquid at the temperature of the subject or object to which the nano-elements are to be administered. They can also be liquid at room temperature, i.e., having a melting temperature of at most 25° C., at most 20° C., at most 15° C., or at most 10° C. However, this is not essential and SFAs may have melting temperatures higher than 25° C. (e.g., quaternary fatty amines).

The hydrophobic tail, which typically comprises between 5 and 40 carbon atoms, renders the SFA insoluble in a polar carrier, the SFAs being for instance water-insoluble, i.e., having a solubility of 5 wt. % or less, 4 wt. % or less, 3 wt. % or less, 2 wt. % or less, 1 wt. % or less, 0.5 wt. % or less, or 0.1 wt. % or less by weight of the liquid.

Suitable SFAs include: fatty amines, fatty acids and metal salts of sulfonates. Combinations of such SFAs may also be used.

Fatty amines suitable as SFAs include primary, secondary, tertiary or quaternary C₈-C₂₂ straight, branched, or cyclic, saturated or unsaturated, aliphatic amines. Comprehensive lists of fatty amines can be found in chemical databases, the following compounds being only provided for illustration of each class. Stearylamine (C₁₈H39N) is an example of a straight, saturated, aliphatic primary alkyl amine (of general formula R-NH2), iso-stearylamine being a branched counterpart and oleylamine (C₁₈H37N) differing by including one unsaturated bond in an alkyl chain of same length. Dinonylamine is a secondary amine (of general formula R1—NH-R2) and trioctylamine is a tertiary amine (N—R1, R2, R3). Quaternary fatty amines can be quaternary ammonium salts or cationic fatty imidazolines, the latter illustrating fatty amines including a heterocyclic ring. In some embodiments, primary, secondary, tertiary or quaternary C₈-C₂₂ amines. In some embodiments, the fatty amines have a C₈-C₂₀ or a C₈-C₁₈ alkyl chain.

Primary amines provide an advantage with respect to the charge they may contribute to the shell, as compared to secondary and tertiary amines included at a similar concentration. Suitable primary amines include: oleyl amine, octyl amine, cocamidopropyl dimethylamine, undecylamine, dodecylamine, stearylamine and iso-stearylamine). Secondary and tertiary amines can be selected from: ethoxylated oleyl amine (such as oleyl bis-(2-hydroxyethyl) amine, commercially available as Genamin® O 020 Special), N,N-dimethyl-dodecylamine, alkyl (C₁₂-C₁₆) dimethylamines, avocadamidopropyl dimethylamine, laurylamine dipropylenediamine, stearyl dimethyl amine, stearamidopropyl dimethylamine, dinonylamine and trioctylamine. Quaternary amines can be quaternary ammonium salts such as cetrimonium chloride. In a particular embodiment, the fatty amine is selected from the group comprising: oleyl bis-(2-hydroxyethyl)amine, N,N-dimethyl-dodecylamine, oleyl amine, octyl amine and cetrimonium chloride.

Fatty acids suitable as SFAs include mono-or di-C₅-C₄₀, C₆-C₃₀, C₈-C₂₂, C₈-C₂₀, or C₈-C₁₈ acids, such as valeric acid, caproic acid, caprylic acid, lauric acid, linoleic acid, linolenic acid, myristic acid, oleic acid, palmitic acid, stearic acid and isostearic acid. In a particular embodiment, the fatty acid is caprylic acid.

Metal salts of sulfonates may also serve as SFAs, wherein the metal counterion can be selected from: barium, calcium, magnesium or sodium, and the sulfonate bears a fatty residue such as an alkyl aryl or petroleum hydrocarbon chain containing between 20 and 30 carbon atoms. Suitable metal salts of sulfonates include: barium alkyl aryl sulfonate, calcium alkyl aryl sulfonate, magnesium alkyl aryl sulfonate, sodium alkyl aryl sulfonate, barium petroleum sulfonate, calcium petroleum sulfonate, magnesium petroleum sulfonate, and sodium petroleum sulfonate.

The temperatures used in preparing the core-shell nano-elements of the present invention should preferably be below the degradation point of the SFAs or of any other components participating in the preparation of the nano-elements. For instance, at high temperatures, a fatty amine may be prone to degradation, resulting in amine loss (due to amidation by oxidation of the amine). Analysis of such degradation can be performed by placing the fatty amine at a tested temperature for a sufficient amount of time, e.g., 24 hours, and then measuring the amine value by routine analysis using standard methods, such as described for instance in ASTM D 2074-07. By way of non-limiting example, the amine value can be assessed by titration of the fatty amine (having been subjected to the tested temperature) with hydrochloric acid, the amine value corresponding to the volume of 0.1N HCl in milliliters needed to neutralize 10 g of product. The obtained result is then compared to the amine value of the same fatty amine in natural, non-heated form, which is provided by the manufacturer, or can be independently measured as described above. Any reduction in the value of the amine number of the heated fatty amines may suggest the degradation of the amines at the tested temperature (such temperature being referred to as the “degradation point” of the fatty amine). Similarly, SFAs being fatty acids may undergo decarboxylation at high temperatures, resulting in the elimination of their chargeable acidic groups. Decarboxylation can be analyzed in a similar manner as described above for fatty amines, adapted to fatty acids. The analysis may include placing the fatty acid at the tested temperature for a sufficient amount of time, followed by measuring the acid value by standard methods (such as by titrating with a base until neutralization and calculating the acid value as described in e.g., ASTM D1980-87). The measured acid value can then be compared to the value reported by the fatty acid manufacturer to confirm or refute that decarboxylation has occurred. More generally and considering that the function of interest with respect to the SFAs is their chargeability, a degradation point of the SFAs is a temperature above which the chargeability of the SFAs in presence of water is significantly damaged.

To enable the formation of the shell, the SFAs must be mixed with the WITCs, their miscibility with the WITC combined with their insolubility in the polar carrier (e.g., the SFAs being water-insoluble), together with the conditions elected to form the nano-elements allowing the proper orientation of their tails and heads and their respective partition between the core and the shell of the nano-elements, without leaching out of the SFAs into the liquid medium. Were materials seemingly similar to the present SFAs added to nano-particles following their formation, a shell would not be formed thereby, such materials at most serving as dispersants found in a liquid medium without partial entrapment in the particles. In order to serve as dispersants, such non-shell-forming materials typically have some degree of solubility in the polar-carrier, in contrast with the SFAs.

As other components of the present nano-elements, the SFAs may, besides forming a chargeable shell, serve additional purposes.

Some SFAs (e.g., oleyl amine) may act as plasticizing agents, capable of reducing the native viscosity of the WITC of the core and facilitating its processing and incorporation as nano-elements into the present compositions. In so doing, or alternatively, the SFAs may serve as “solubilizers” capable of increasing the miscibility of other materials (e.g., carrier-insoluble active agents) within the WITC.

Alternatively, or additionally, the SFAs (e.g., oleyl bis-(2-hydroxyethyl) amine) may also serve as surfactants, suitable to stabilize the core-shell nano-elements within the polar carrier. In some embodiments, the SFAs' portion exposed in the shell may enable charging of the core-shell nano-elements to such an extent that the elements can remain stably dispersed in the composition in absence of a dedicated surfactant externally provided in the liquid phase (such as demonstrated in Examples 1-II and 2-II). In such a case, the core-shell nano-elements can be viewed as “self-emulsifying” or “self-emulsified”.

Alternatively, or additionally, the SFAs may further serve as carrier-insoluble active agents, not only forming the shell of the core-shell nano-elements but also exerting their own activity (e.g., as germicides or bactericides).

### Compositions

Having reviewed the various components that may be used in the present compositions, suitable concentrations or respective proportions shall be provided below. It is to be noted that while compounds have been for simplicity categorized according to their main role in the present nano-elements, such functions are not exclusive one of the other, and some of the components according to the present teachings can serve in more than one role. The predominance of one role over the other may depend upon a material inherent potency in the respective fields of activities considered, but also on the relative presence of the material in the composition. For instance, a material deemed a carrier if constituting a significant enough part (e.g., more than 20 wt. %) of the liquid phase, may be considered to fulfil a distinct function if in a relatively lower amount, such amount being more adapted to its secondary roles. Thus, when referring to the concentration of a certain component in the nano-elements or in compositions comprising the same, the information refers only to dedicated compounds intentionally added to serve this role, excluding compounds having a different primary role.

In some embodiments, the concentration of the WITC (or combination thereof if WITCs) in the nano-elements is within the range of 0.1 wt. % to 99.9 wt. % by total weight of the nano-elements. In some embodiments, the concentration of the WITC(s) is within the range of 1 wt. % to 99 wt. %, 1 wt. % to 90 wt. %, 5 wt. % to 80 wt. %, 10 wt. % to 50 wt. %, or 15 wt. % to 40 wt. % by total weight of the nano-elements.

In some embodiments, the concentration of the WITC(s) in the composition or the medication prepared therewith is at least 0.1 wt. %, at least 0.5 wt. %, at least 1 wt. %, at least 2wt. %, at least 3 wt. %, or at least 4 wt. % by total weight of the composition. In other embodiments, the WITC(s) concentration is at most 30 wt. %, at most 25 wt. %, at most 20 wt. %, at most 15 wt. %, at most 13 wt. %, at most 10 wt. % or at most 8 wt. % by total weight of the composition. In other embodiments, the concentration of the WITC(s) is within the range of 0.1 wt. % to 30 wt. % by total weight of the composition, preferably in the range of 0.5 wt. % to 25 wt. %, 1 wt. % to 20 wt. %, or of 1.5 wt. % to 15 wt. %, 0.5 wt. % to 13 wt. %, 1 wt. % to 10 wt. %, 2 wt. % to 10 wt. %, 3 wt. % to 10wt. %, 4 wt. % to 10 wt. %, or of 4 wt. % to 8 wt. %.

The active agents are generally present in the medications of the present invention in an amount effective to treat the intended condition by one or more administration to a subject or object in need thereof. Considering for illustration medications to be administered to living subjects, the quantity or concentration of active agent(s) is often referred to as a therapeutically effective amount, which depends inter alia on the nature of the active agents, the concentration to be achieved at the targeted site to achieve a therapeutic response without significant adverse effects, the route of administration, the length of treatment, the regimen of administration, the absorption and excretion rates of the active agents, and like factors known to one of ordinary skill in the art of pharmacology. As appreciated, even for a specific active agent, some of the afore-said factors may vary according to circumstances. Notably, dosage values may vary according to the severity of the condition to be alleviated. Generally, dosage regimens should be adjusted over time according to the individual need and the professional judgment of the person administering or supervising the administration of the medication. The medication may be administered once or may be divided into a number of smaller doses to be administered at varying intervals of time. Hence, when the medication prepared according to the present teachings is a pharmaceutical product, an appropriate amount of active agents, e.g. an amount suitably providing for a desired therapeutic effect without adverse ones, can be in a range of values which for each active agent are known to one of ordinary skill in the art or can be optimized by conducting pertinent trials (e.g., bioequivalence studies).

In some embodiments, the concentration of the carrier-insoluble active agent(s), if present in the nano-elements, is within the range of 0.1 wt. % to 99.9 wt. %, within the range of 0.5 wt. % to 85 wt. %, within the range of 1 wt. % to 85 wt. %, within the range of 2 wt. % to 70 wt. %, within the range of 3 wt. % to 55 wt. %, within the range of 5 wt. % to 45 wt. %, within the range of 5 wt. % to 35 wt. %, within the range of 10 wt. % to 30 wt. %, within the range of 15 wt. % to 25 wt. %, within the range of 0.1 wt. % to 25 wt. %, within the range of 0.5 wt. % to 25 wt. %, or within the range of 1 wt. % to 25 wt. % by total weight of the nano-elements.

In some embodiments, the concentration of the carrier-insoluble active agent(s), if present in the composition, is within the range of 0.01 wt. % to 30 wt. % by total weight of the composition, preferably in the range of 0.05 wt. % to 25 wt. %, 0.1 wt. % to 20 wt. %, 0.5 wt. % to 15 wt. %, 1 wt. % to 12.5 wt. %, 2 wt. % to 10 wt. %, 3 wt. % to 10 wt. %, or of 5 wt. % to 10 wt. %.

In some embodiments, the carrier-soluble active agent(s), if present, is added to the nano-suspension at a concentration of up to 200 wt. % by weight of the WITC(s), preferably within the range of 0.1 wt. % to 170 wt. %, within the range of 1 wt. % to 150 wt. %, within the range of 5 wt. % to 100 wt. %, within the range of 5 wt. % to 50 wt. %, within the range of 5 wt. % to 40 wt. %, or within the range of 10 wt. % to 20 wt. %.

The non-volatile liquid(s) (or a combination thereof) can be included for plasticizing at a weight ratio of at least 1:200, at least 1:20, at least 1:10, at least 1:5, or at least 1:3, at least 1:1, at least 2:1, or at least 3:1, with respect to the weight of the WITC(s) to be plasticized therewith. In some embodiments, the weight ratio of the non-volatile liquid(s) to the WITC(s) is of at most 100:1, at most 50:1, at most 20:1, at most 10:1, or at most 5:1.

In some embodiments, the concentration of the non-volatile liquid(s) is at least 1 wt. %, at least 5 wt. %, at least 10 wt. %, or at least 20 wt. % by weight of the nano-elements. In some embodiments, the concentration of the non-volatile liquid(s) is at most 99 wt. %, at most 90 wt. %, at most 80 wt. %, at most 70 wt. %, or at most 60 wt. % by total weight of the nano-elements. In other embodiments, the concentration of the non-volatile liquid(s) is within the range of 1 wt. % to 99 wt. % by total weight of the nano-elements, preferably in the range of 5 wt. % to 90 wt. %, 10 wt. % to 80 wt. %, 20 wt. % to 70 wt. %, or 20 wt. % to 60 wt. %.

In some embodiments, the concentration of the non-volatile liquid(s), if present in the composition, is at least 0.1 wt. %, at least 0.5 wt. %, at least 1 wt. %, or at least 5 wt. % by weight of the composition. In other embodiments, the concentration of the non-volatile liquid(s) is at most 50 wt. %, at most 45 wt. %, at most 40 wt. %, at most 35 wt. %, at most 30 wt. %, at most 25 wt. %, at most 22.5 wt. %, or at most 20 wt. % by weight of the composition. In other embodiments, the concentration of the non-volatile liquid(s) is within the range of 0.1 wt. % to 50 wt. % by total weight of the composition, preferably in the range of 0.1 wt. % to 45 wt. %, 0.1 wt. % to 40 wt. %, 0.5 wt. % to 35 wt. %, 0.5 wt. % to 30 wt. %, 0.5 wt. % to 25 wt. %, 1 wt. % to 22.5 wt. %, or of 5 wt. % to 20 wt. %.

In some embodiments, the concentration of the SFA (or combination thereof if SFAs) in the nano-elements is within the range of 1 wt. % to 99 wt. %, 1 wt. % to 90 wt. %, 5 wt. % to 80wt. %, 10 wt. % to 50 wt. %, or 15 wt. % to 40 wt. % by total weight of the nano-elements.

In some embodiments, the concentration of the SFA(s) in the composition is at least 0.1 wt. %, at least 0.5 wt. %, at least 1 wt. %, or at least 1.5 wt. %, by total weight of the composition. In other embodiments, the concentration of the SFA(s) is at most 30 wt. %, at most 25 wt. %, at most 20 wt. %, or at most 15 wt. % by total weight of the composition. In other embodiments, the SFA(s) concentration is within the range of 0.1 wt. % to 30 wt. % by total weight of the composition, preferably in the range of 0.5 wt. % to 25 wt. %, 1 wt. % to 20 wt. %, or of 1.5 wt. % to 15 wt. %.

In some embodiments, the polar carrier (e.g., water) is present in a composition or medication being a liquid dosage form within the range of 30 wt. % to 90 wt. %, 30 wt. % to 80 wt. %, 40 wt. % to 70 wt. %, or 30 wt. % to 60 wt. % by total weight of the composition or medication. As mentioned, the nano-elements may alternatively be isolated to prepare medications being a dry dosage form, hence substantially devoid of any liquid vehicle, whether polar or not.

In some embodiments, the concentration of the non-ionic or amphoteric “charge neutral” surfactant(s), if present in the nano-elements, is within the range of 0.1 wt. % to 50 wt. %, within the range of 1 wt. % to 50 wt. %, within the range of 5 wt. % to 50 wt. %, within the range of 10 wt. % to 50 wt. %, within the range of 15 wt. % to 45 wt. %, or within the range of 20 wt. % to 40 wt. % by total weight of the nano-elements. These concentrations refer to dedicated surfactants intentionally added for this purpose and do not include the presence of any other material capable of also serving as surfactants, such as some SFA(s).

In some embodiments, the combined concentration of the surfactants (including, for instance, the emulsifiers and/or hydrotropes only found in the liquid phase), if present in the composition, is at least 0.1 wt. %, at least 0.5 wt. %, at least 1 wt. %, at least 5 wt. %, at least 6 wt. %, at least 7 wt. %, or at least 8 wt. % by total weight of the composition. In other embodiments, the combined concentration of the surfactants is at most 60 wt. %, at most 40 wt. %, at most 35wt. %, at most 30 wt. %, at most 25 wt. %, at most 20 wt. % or at most 15 wt. % by total weight of the composition. In other embodiments, the combined concentration of the surfactants is within the range of 0.1 wt. % to 60 wt. %, within the range of 0.5 wt. % to 60 wt. %, within the range of 1wt. % to 60 wt. %, 5 wt. % to 40 wt. %, 6 wt. % to 30 wt. %, 7 wt. % to 25 wt. %, 8 wt. % to 20 wt. %, or 5 wt. % to 15 wt. % by total weight of the composition. Alternatively, the composition is substantially devoid of a dedicated surfactant, the concentration of one or more surfactants being less than 0.1 wt. % by total weight of the composition.

In some embodiments, the concentration of the emulsifier(s), if present in the composition, is at least 0.01 wt. %, at least 0.1 wt. %, at least 0.5 wt. %, at least 1 wt. %, at least 3 wt. %, or at least 5 wt. % by total weight of the composition. In other embodiments, the concentration of the emulsifier(s) in the composition is at most 60 wt. %, at most 50 wt. %, at most 40 wt. %, at most 30 wt. %, at most 25 wt. %, or at most 20 wt. % by total weight of the composition. In other embodiments, the concentration of the emulsifier(s) in the composition is within the range of 0.01 wt. % to 60 wt. %, 0.1 wt. % to 50 wt. %, 0.5 wt. % to 40 wt. %, 1 wt. % to 30 wt. %, 3 wt. % to 25 wt. %, or 5 wt. % to 20 wt. % by total weight of the composition.

In some embodiments, the concentration of the hydrotrope(s), if present in the composition, is at least 0.01 wt. %, at least 0.05 wt. %, at least 0.1 wt. %, at least 0.5 wt. %, or at least 1 wt. % by total weight of the composition. In other embodiments, the hydrotrope(s) concentration in the composition is at most 60 wt. %, at most 50 wt. %, at most 40 wt. %, at most 30 wt. %, at most 25 wt. %, at most 20 wt. %, at most 15 wt. %, or at most 10 wt. % by total weight of the composition. In some embodiments, the hydrotrope(s) concentration in the composition is within the range of 0.01 wt. % to 60 wt. %, 0.05 wt. % to 50 wt. %, 0.1 wt. % to 40 wt. %, 0.1 wt. % to 30 wt. %, 0.5 wt. % to 25 wt. %, 1 wt. % to 20 wt. %, or 1 wt. % to 10 wt. % by total weight of the composition.

While each prospective component of the nano-elements may have a concentration in relatively wide ranges of values, it is clarified that each component present in the nano-elements, or the compositions or medications comprising the same, is at a respective concentration such that in total all components sum up to 100 wt. %.

Preferably, the aforesaid ingredients are approved for use at the envisioned concentrations for the diagnosis or treatment of ailments in living subjects or of other deleterious conditions affecting objects. For instance, they do not lead to allergic reactions, or any other acute or chronic adverse effect, whether administered to an animal subject or to an edible plant that may be consumed by the animals. Moreover, all ingredients need be compatible one with another, such compatibility being as described above. As readily understood, this principle of compatibility, which can be affected not only by the chemical identity of the materials, but by their relative proportions according to the intended use, should preferably guide the selection of all materials necessary for the compositions disclosed herein.

### Method of Preparation

The nano-elements having a core made of a water-insoluble thermoplastic compound (WITC), in particular of a water-insoluble thermoplastic polymer (WITP), being dispersible as a nano-suspension in a polar liquid and further including an active agent in a core or shell of the nano-elements can be prepared as follows. The properties and characteristics of the materials used in the present method are as described above for each of the materials suitable for the present medications. Notably, the nano-elements including the WITCs and the active agents are substantially free of VOC compounds and contain less than 2 wt. % or even less than 0.2 wt. % of VOCs or a blend thereof per weight of the nano-elements.

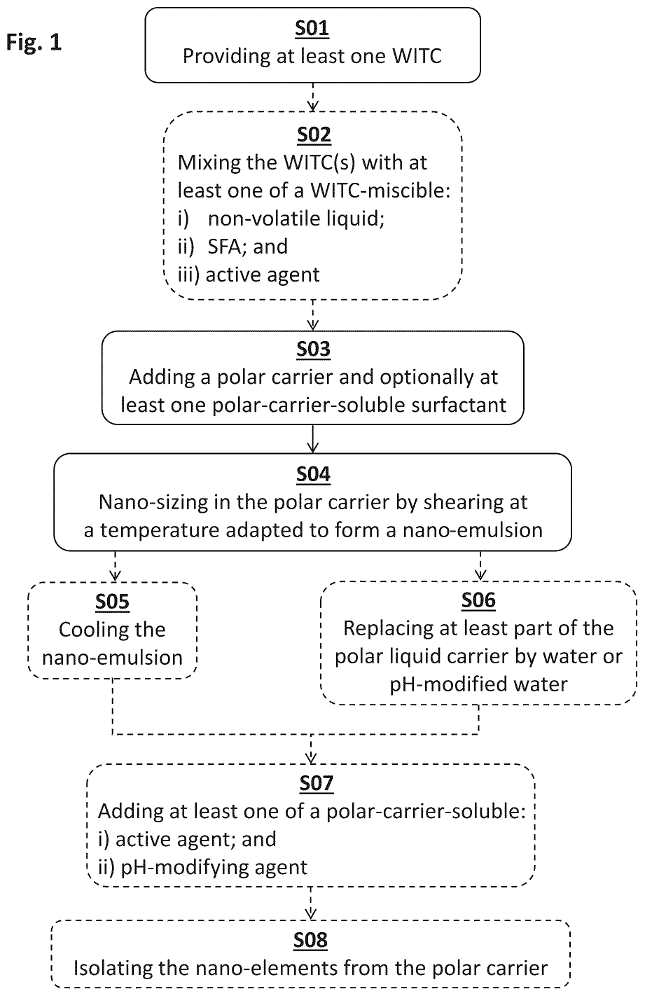

The steps of the present method are briefly displayed in FIG. 1 and further detailed hereinbelow, a step having a dashed contour being optional.

In a first step (S01) of the method, at least one WITC (e.g., at least one WITP) is provided.

In a second step (S02) of the method, the WITC(s) can be mixed with additional materials, typically miscible within the WITC(s), such as at least one of: i) non-volatile liquid(s); ii) SFA(s) (when core-shell nano-elements having a chargeable shell are desired); and iii) WITC-miscible/polar-carrier-insoluble active agent(s) (when active agents are desired within the cores of the nano-elements).

The addition of the non-volatile liquid(s) is optional when the viscosity of the WITC(s) provided in S01 is sufficiently low for further processing (e.g., 10⁷ mPa·s or less, as measured at at least one temperature of between 20° C. and 80° C., and at a shear rate of 10 sec⁻¹). Alternatively, any one of the other prospective components of the core (e.g., active agents or SFAs) can also serve, when suitable for this purpose, as agents sufficiently plasticizing the WITC(s) so as to render the addition of non-volatile liquid(s) optional.

If intentional plasticizing of the WITC(s) is desired, its mixing with the non-volatile liquid(s) (and any additional agents possibly serving as plasticizing agents) can be performed at any mixing temperature and/or mixing pressure suitable for such compounding.

The temperature at which the mixing (which may encompass the plasticizing of the WITC) is performed is typically selected according to temperatures characterizing the substances involved in the process, for instance, by taking into account a first Ts, Tm and/or Tg characterizing the WITC(s) and, optionally, a Tb of the SFA(s) (referred to as Tb_{SFA}) and/or a Tb of the non-volatile liquid(s) (referred to as Tbᵢ) and/or the degradation point(s) of the SFA(s), when applicable (e.g., when the polar head of the SFA loses its chargeability if heated above a certain point). As previously detailed, a mixing temperature would suitably be higher (e.g., by at least 5° C., at least 10° C., at least 15° C., or at least 20° C.) than at least one of the characterizing temperatures of the WITC(s) and preferably lower (e.g., by at least 5° C., at least 10° C., at least 15° C., or at least 20° C.) than the lowest of the boiling temperatures of the non-volatile liquids and/or SFAs and/or the degradation point of the SFAs, at a pressure the mixing step is performed. This recommended upper limit of mixing temperature is however not essential as long as the selected mixing temperature does not significantly boil away or degrade any of the materials to be mixed with the WITCs so as to form the cores of the nano-elements. Hence, in some cases, the mixing temperature can even be at Tbₗ, Tb_{SFA}, or the SFA degradation point (as applicable) or higher, if the step is brief enough and/or the non-volatile liquid in sufficient excess and/or the mixing performed in a chamber sufficiently sealed to limit evaporation of liquid components/favor their condensation back to the mixture.

One can readily appreciate that a change in the properties of the substance reflected by these temperatures dropping from first to second values can alternatively take place at a lower mixing temperature or a higher mixing temperature, if the pressure in a sealed chamber hosting the mixing process ensuring plasticization of the WITC(s) were to be accordingly reduced or increased. Therefore, while in the description of a method suitable for the preparation of a composition used according to the present teachings, reference can be made to specific temperatures and duration of times assuming the process is carried out under standard atmospheric pressure, such guidance should not be viewed as limiting, and all temperatures and durations achieving a similar outcome with respect to the behavior of the plasticized WITC(s) and/or the possible chargeability of core-shell nano-elements in view of the activity of the SFAs are encompassed.

It is noted in this context, when the WITC is a WITP, that while the Tm and/or Tg of a polymer may set relatively clear temperatures below and above which a polymer may display a distinct behavior, this typically does not apply to the Ts. In view of their viscoelastic properties, a polymer or a plasticized polymer may remain “sufficiently solid” even at a temperature moderately higher than its formal softening point.

The mixing or plasticizing of the WITCs and materials miscible therewith can be performed under a variety of conditions, such as elevated temperatures (i.e., 30° C. or more, e.g., at 40° C. or more, at 50° C. or more, at 60° C. or more, at 75° C. or more, or at 90° C. or more) and/or elevated pressure (i.e., 100 kPa or more, e.g., at 125 kPa or more, 150 kPa or more, 175 kPa or more, 200 kPa or more, 250 kPa or more, or 300 kPa or more). As the mixing step often achieves at least some plasticizing of the WITC, it may also be referred to as a plasticizing step, the aforesaid temperatures and/or pressures typically accelerating the plasticizing process (i.e., shortening the duration of the plasticizing period) or enabling a desired modification of a boiling temperature Tbₗ or Tb_{SFA} at which the non-volatile liquid or the SFA, if present, might evaporate. As mixing at elevated pressure increases Tbₗ and/or Tb_{SFA}, the range of temperatures at which plasticizing could be performed can be accordingly widened. Conversely, plasticizing the WITC under conditions less favorable than arbitrarily set to assess the ability of a WITC to be plasticized by a specific agent, such as at a temperature of less than 50° C. and/or a reduced pressure of less than 100 kPa, may prolong the plasticizing process, if desired. The ability of a WITC to be plasticized by a particular plasticizing agent may be assessed under any one of the above temperature or pressure conditions.

Mixing of the WITC(s) with agents promoting its plasticizing and/or modifying the contents of the cores by agitating the mixture can also shorten the plasticizing/mixing period, such agitating additionally ensuring that all parts of the WITC(s) are plasticized and/or all parts of the intended cores are blended in a relatively uniform manner, the plasticized WITC(s) behaving reasonably homogeneously with respect to subsequent steps of the method and results expected therefrom. If excess of the non-volatile liquid is used during the plasticizing process, it can be optionally removed before proceeding to following step(s). When the materials to be plasticized have a relatively high viscosity, the mixing step can also be referred to as compounding, and the mixing equipment can be accordingly selected.

The duration of plasticizing will inter alia depend on the WITC(s) being plasticized, the non-volatile liquid(s) being used, the plasticizing conditions (e.g., temperature, pressure, and/or agitation), and the desired extent of plasticizing. The plasticizing period can be of at least 1 minute and at most 4 days. The plasticizing conditions and its duration need also be suitable to the active agent(s) and optional SFA(s) to be incorporated into the WITC(s) being plasticized.

The mixing may be performed by any method known to the skilled artisan, such as: sonication, using a double jacket planetary mixer or a high shear mixer, etc. When the materials being mixed have a relatively high viscosity, the mixing step can be performed with a two-roll mill, a three-roll mill, an extruder and such type of equipment. In a particular embodiment, the mixing is performed by sonication.

Additional WITC-miscible components may be added, if needed, such as: polar-carrier-insoluble charge neutral non-ionic or amphoteric surfactant(s), pH-modifying agent(s) and any other desirable additives.

When a WITC-miscible/polar-carrier-insoluble active agent(s) is also included, it can be combined with the WITC(s) either a) prior to heating the WITC(s) and/or mixing with optional non-volatile liquid(s), SFA(s), charge neutral surfactant(s) and/or any other desirable ingredient miscible therewith, or b) after any such heating and/or mixing, once the WITC(s) is at least partially softened or plasticized by heat and/or ingredients mixed therewith.

In a third step (S03) of the method, the WITC of step S01 or mixture comprising it obtained in optional step S02 is combined with at least one polar carrier. At least one surfactant can be added to the polar carrier, if desired, at this step, the surfactant being a relatively polar emulsifier or a hydrotrope. Additional materials which are soluble in the polar carrier could also be added at this step (e.g., active agents that may form a shell around the cores to be formed by materials of previous steps) but may equally be introduced after the following nano-sizing step.

The liquid mixture obtained in step S03 is nano-sized in a fourth step (S04) to form a nano-suspension, whereby nano-elements of WITC(s) containing any desirable WITC-miscible and polar-carrier-insoluble materials, are dispersed in a polar liquid including the polar carrier optionally combined with other polar materials.

As the nano-sizing is typically performed by applying shear at a relatively elevated temperature, the nano-elements are generally nano-droplets during that step and the resulting nano-suspension is a nano-emulsion. The nano-emulsion can be obtained by nano-sizing the mixture of desired materials by any method capable of shearing the WITC(s) (whether plasticized or not, or including additional compounds), the shearing method being selected from the group comprising: sonication, milling, attrition, high pressure homogenization, high shear mixing and high shear microfluidization. In a particular embodiment, the nano-sizing is performed by sonication.

The nano-sizing is performed at a shearing temperature that is at least equal to at least one of the first Ts, Tm and Tg of the WITC(s), at least equal to at least one of the second Ts, Tm and Tg of the plasticized and/or mixed WITC, and can be, in some embodiments, at least 5° C. higher, at least 10° C. higher, or at least 15° C. higher than the highest characterizing temperature of the WITC mix being sheared. However, while this is not essential if the shearing step is brief enough and/or the polar liquid in sufficient excess, the shearing temperature should preferably prevent significant amounts of the liquid phase being boiled away (and avoid a significant degradation of the SFA(s), if present). In some embodiments, the nano-sizing temperature at which shearing is performed does not exceed the boiling temperature of the liquid phase in which the shearing is being performed (or of any other liquid the evaporation of which should be prevented) and the degradation point of the SFA(s) (or the temperature withstood by any other material the thermal degradation (e.g., deactivation, destruction, etc.) of which should be prevented). Thus, the shearing temperature is generally lower than the lowest of the Tb of the polar carrier(s) (referred to as Tbc) and the degradation point(s) of the material(s) mixed with the WITC(s) at a pressure the nano-sizing step is performed. For instance, when the polar carrier is water, the shearing temperature can be selected to be lower than 95° C., lower than 90° C., lower than 85° C., or lower than 80° C., assuming the nano-sizing is performed at atmospheric pressure. However, if the nano-sizing were to be performed at an elevated pressure, the Tbc of the polar carrier would be raised, and the shearing temperature could be accordingly increased. Still illustrating with water, while its Tb is 100° C. at about 100 kPa, this boiling temperature rises to 120° C. at about 200 kPa, in which case the nanosizing temperature not to be exceeded could be of up to 115° C. As mentioned, these upper limits, while preferred, are not essential, as any boiling away or degradation of a part, of e.g., the polar carrier, the active agent(s), the surfactant(s) or the SFA(s) could be prevented at even higher temperatures if the step is brief enough, and/or the polar carrier in sufficient excess and/or the nano-sizing is performed in a chamber sufficiently sealed to limit its evaporation/favor its condensation back to the nano-suspension.

At shearing temperatures in this range of higher than Ts, Tm or Tg of the WITC(s) and optionally lower than Tbc of the liquid carrier or degradation temperatures of any of the materials present, the WITC(s), and in particular the WITP(s), can completely melt and the nano-sizing process can be considered as “melt nano-emulsification”.

In some embodiments, at least 50% of the total number (D_{N}50) or volume (D_{V}50) of the nano-elements (including core and core-shell nano-elements, being nano-droplets or nano-particles) formed in this nano-sizing step have a hydrodynamic diameter of up to 1,000 nm or less, 750 nm or less, 500 nm or less, or 250 nm or less. In particular embodiments, the D_{N}50 or D_{V}50 of the nano-elements is up to 200 nm, up to 150 nm, up to 100 nm, up to 90 nm, up to 80 nm, or up to 70 nm. In some embodiments, the median diameter of the nano-elements is at least 5 nm, at least 10 nm, at least 15 nm, or at least 20 nm. Advantageously, such values are applicable as determined by the number of the nano-elements.

As readily appreciated, depending on the temperatures characterizing the materials of the nano-elements and/or on the temperature at which measurements may be performed, the nano-elements can either be relatively liquid nano-droplets or relatively solid nano-particles, as the temperature is reduced. The size of the nano-particles at room temperature is commensurate with the size of the nano-droplets at a higher temperature or slightly more compact, their median diameter not exceeding 1,000 nm, and preferably not exceeding 200 nm.

In some embodiments, the size of the nano-particles or nano-droplets is determined by microscopy techniques, as known in the art (e.g., by Cryo TEM). In some embodiments, the size of the nano-elements is determined by Dynamic Light Scattering (DLS). In DLS techniques the particles are approximated to spheres of equivalent behavior and the size can be provided in term of hydrodynamic diameter. DLS also allows assessing more readily the size distribution of a population of nano-elements.

Distribution results can be expressed in terms of the hydrodynamic diameter for a given percentage of the cumulative particle size distribution, either in terms of numbers of particles or volumes, and are typically provided for 10%, 50% and 90% of the cumulative particle size distribution. For instance, D50 refers to the maximum hydrodynamic diameter below which 50% of the sample volume or number of particles, as the case may be, exists and is interchangeably termed the median diameter per volume (D_{V}50) or per number (D_{N}50), respectively, and often more simply the average diameter.

In some embodiments, the nano-elements of the disclosure have a cumulative particle size distribution of D90 of 500 nm or less, or a D95 of 500 nm or less, or a D97.5 of 500 nm or less or a D99 of 500 nm or less, i.e., 90%, 95%, 97.5% or 99% of the sample volume or number of particles respectively, have a hydrodynamic diameter of no greater than 500 nm.

Any hydrodynamic diameter having a cumulative particle size distribution of 90%, or 95%, or 97.5%, or 99% of the particles' population, whether in terms of number of particles or volume of sample, may be referred to hereinafter as the “maximum diameter”, i.e., the maximum hydrodynamic diameter of particles present in the population at the respective cumulative size distribution. It is to be understood that the term “maximum diameter” is not intended to limit the scope of the present teachings to nano-particles having a perfect spherical shape.

The nano-particles or nano-droplets may, in some embodiments, be uniformly shaped and/or within a symmetrical distribution relative to a median value of the population and/or within a relatively narrow size distribution.

A particle size distribution is said to be relatively narrow if at least one of the following conditions applies:

the difference between the hydrodynamic diameter of 90% of the nano-elements and the hydrodynamic diameter of 10% of the nano-elements is equal to or less than 250 nm, equal to or less than 200 nm, equal to or less than 150 nm, or equal to or less than 100 nm, or equal to or less than 50 nm, which can be mathematically expressed by: (D90−D10)≤250 nm and so on;

the ratio between a) the difference between the hydrodynamic diameter of 90% of the nano-elements and the hydrodynamic diameter of 10% of the nano-elements; and b) the hydrodynamic diameter of 50% of the nano-elements, is no more than 2.5, no more than 2.0, or no more than 1.5, or even no more than 1.0, which can be mathematically expressed by: (D90−D10)/D50≤ 2.5 and so on; and

the polydispersity index of the nano-elements is equal to or less than 0.5, equal to or less than 0.4, or equal to or less than 0.3, or equal to or less than 0.2, which can be mathematically expressed by: PDI =02/d2 ≤ 0.5 and so on, wherein o is the standard deviation of the particles distribution and dis the mean size of the particles, the PDI optionally being equal to 0.01 or more, 0.05 or more, or 0.1 or more.

The PDI information is generally readily obtained from the instrument used to measure the hydrodynamic diameter of the nano-particles.

In a fifth step (S05) of the method, the nano-emulsion obtained in step S04 may optionally be actively cooled down to a temperature below the first Tm, Ts or Tg of the WITC(s) (or the second Tm, Ts or Tg of the nano-elements, when lower), to accelerate the relative solidification of the nano-elements, if desired in manufacturing. Such cooling can be actively achieved by refrigerating the nano-suspension (e.g., placing in a coolant having a desired low temperature), by subjecting the nano-suspension to ongoing agitation to accelerate heat dissipation (and incidentally maintain proper dispersion of the nano-droplets as they cool down), or by combining both approaches. This cooling step is optional, as the nano-emulsions may be allowed to passively cool down without any agitation upon termination of nano-sizing. The nano-emulsions may also be passively cooled when combined with water (pH-modified water) and/or a polar-carrier-soluble active agent in subsequent optional steps, provided they are at sufficiently low temperature that would allow cooling of the nano-emulsion. Despite cooling, if performed during the manufacturing process, the WITCs (or plasticized WITCs) may remain in a liquid form as nano-droplets, or may readily convert back to nano-droplets once administered to a subject having a body temperature of more than 30° C., such temperatures typically being respectively of more than 32° C. and 35° C. in mammals.

When the polar carrier added in step S03 for the sake of nano-sizing is non-aqueous, the method may optionally include a sixth step (S06), whereby replacement of at least part of the polar carrier by water or pH-modified water is carried out. As previously described, when the polar carrier is water or a mixture thereof with a non-aqueous polar carrier, such replacement step may not be necessary.

Alternatively, an aqueous pH-modified solution of the polar carrier may be utilized in the fourth step of the method (S04), resulting in an environment promoting the charging of the nano-elements, whereby a possible masking effect of the polar carrier is at least partially avoided.

The foregoing nano-elements and compositions comprising them, assuming that the intended active agent(s) are all WITC-miscible and incorporated therein during previous steps, may then be formulated into medications (pharmaceutical or agrochemical) suitable for administration to a subject or an object to be treated therewith, the formulation of the medication being according to the NES route (e.g., ND route) it is to be administered by so that the active agent(s) may exert their sought effect.

Alternatively, in a further optional seventh step (S07) of the method, a polar-carrier-soluble active agent can be added and dissolved by stirring in the polar carrier. When polar-carrier-soluble active agents are added to core-shell nano-elements (that may optionally contain other active agents in their cores), the soluble active agents can envelop the outer surfaces of the nano-elements, forming a second shell as described above. The second shell may be covalently and/or non-covalently attached to a first shell constituted of chargeable polar moieties of amphiphilic SFAs and/or active agents, said first shell not being covalently attached to the core.

When the core-shell nano-elements have a charge insufficiently strong in the polar carrier to attract compounds of an opposite charge, S07 may further comprise adding a carrier-soluble pH-modifying agent, in an amount suitable to increase the charge of the core-shell nano-elements (e.g., raising a positive charge), while retaining the opposite (e.g., negative) charge of the carrier-soluble active agent and vice versa. Depending on the chargeable groups present on the SFA molecules of the first shell, the pH-modifying agent may be either an acid or a base.

While depicted in the figure as a separate step following cooling of the nano-suspension, whether active (S05) or passive, the optional replacement of at least part of the polar carrier (S06) might alternatively be performed prior to or during cooling.

The addition of the polar carrier-soluble active agent(s) (S07) can also be performed at various steps during the preparation of the composition, depending on the resistance of the active agent to temperatures, mixing or shearing conditions applied at the envisioned step. Relatively resistant active agents can be added i) whilst combining the WITC (and optionally additional WITC-miscible components) with the polar carrier; or ii) whilst nano-sizing the compositions components so as to obtain the core or core-multi-shells nano-elements. Alternatively, the carrier-soluble active agent(s), in particular if shear-sensitive, can be added to the obtained nano-suspension, relatively heat-sensitive agents being preferably combined with the nano-emulsion or nano-dispersion after cooling.

In yet another optional eighth step (S08) of the method, the nano-elements can be isolated from the polar carrier. This step allows the isolated nano-elements to be later combined with suitable excipients, depending on the type of dosage form they are to be incorporated within. For instance, if the dosage form is in a dry form, the isolated nano-elements can be mixed with the adequate excipient to form a dry dosage form (e.g., a tablet or capsule). If, the dosage form is in a liquid form (e.g., to be administered intravenously), such a step may serve to transfer the isolated nano-elements from the polar carrier to a different liquid vehicle adapted to the desired liquid dosage form.

While in the method detailed above, some ingredients have been described as being introduced (or optionally introduced) in the composition at a particular step, this should not be construed as limiting. Some ingredients can be introduced at more than one step and can in practice be introduced step-wisely during the preparation at two separate steps and/or gradually during the performance of a step, the material being added as the step proceeds. For illustration, one or more polar-carrier soluble surfactants can be added during the shearing step, optionally different ones at the beginning and at the end of the step. Thus, the above-described steps can be modified, omitted (e.g., S02, S05, S06, S07 or S08) and additional steps may be included. For instance, the composition may comprise any additive customary to medications or compositions for preparing the same, such as diluents, extenders, binders, lubricants, disintegrating agents, coloring agents, flavoring agents, moisturizers, emollients, humectants, UV-protective agents, thickeners, preservatives, antioxidants, bactericides, fungicides, chelating agents, vitamins and fragrances, to name a few. The nature and concentration of each such conventional compounds, also known as pharmaceutical excipients when considering pharmaceutical compositions, as suitable for each such medications and routes of administration are known to the skilled person and need not be further detailed herein. The additives may be added during steps of the method already described or via new steps. Furthermore, the composition may be further treated (e.g., sterilized, filtered, irradiated, etc.) in accordance with health or agricultural regulations, to make it suitable for its intended uses.

Advantageously, the present method, regardless of the steps involved and the compositions being prepared, does not seek to chemically modify its ingredients, as might have been required for instance to jointly attach them. The absence of such modifications in the present compositions is expected to prevent formation of large particles, having difficulties reaching their target sites due to their size, and/or is believed to prevent an undesirable decrease in the biological activity these ingredients might provide in their native (unmodified) form, assuming they are successfully delivered to their target site.

While for brevity the present compositions are being predominantly described as suitable for the diagnostic and treatment of animal subjects, as relevant for veterinary or human use, they might also serve for the systemic delivery of agents of relevance to the plant realm, such as factors capable of promoting plant growth (e.g., phytohormones or other fertilizers) or capable of reducing deleterious conditions adversely affecting such growth (e.g., pesticides, fungicides, insecticides, etc.).

The compositions used in the present invention may be delivered by any route suitable for their intended use depending on the object, the subject and/or the condition to be treated therewith. Such modes of administration are known and have been exemplified in the foregoing.

Depending on the ND route of administration, the present medications can be prepared in dosage forms selected from a group comprising: solid dosage forms such as cachets, capsules (with hard or soft shells), tablets (including lozenges, sublingual, chewable, dispersible, disintegrating, coated, and effervescent tablets, to name a few), granules, powders, said dosage forms being optionally coated to provide for a modified or prolonged release of the active agents or to provide an accrued resistance to some physiological environments (e.g., being gastro-resistant), some of the foregoing solid dosage forms serving to prepare at the time of intended administration a liquid dosage form (e.g., when effervescent or serving to reconstitute solutions or dispersions at the time they become necessary, and which can be administered in liquid form or as vapors). Solid dosage forms can also be in the form of films, such as oral films, in the form of suppositories or sticks, or be devices impregnated by the medication, such as impregnated pads, intrauterine devices or implants. The dosage forms can alternatively be semi-solid (e.g., creams, foams, pastes or gels) or liquid (e.g., syrups, solutions, dispersions or emulsions), whether as final dosage forms ready to use or as intermediate concentrate for the preparation of the final medication at the time it is to be administered. For illustration, the concentrated dosage forms can be for the preparation of gargles, haemodialysis solutions, rectal solutions (e.g., enemas), solutions for injection (e.g., by IV, IP, IM, SC etc.), infusion, irrigation or instillation. Medications that are to be prepared in final form at the time of administration can be supplied as part of a kit including the materials relevant to said preparation. Solid or liquid medications can be intended for administration by inhalation (e.g., oral or nasal) and be in the form of aerosols or sprays and include, when needed, gases to propel the medication. The dosage form can also be selected in accordance with the organs to be treated and can, taking the eyes for illustration, be eye creams, eye drops, eye gels, eye lotions, eye sprays, eye washes etc. or powders and/or liquids for the reconstitution of foregoing liquid medication by resuspension and/or dilution, the final medication being mixed before administration. Some of the preceding dosage forms illustrated for ocular administration exist also for oral, gingival, dental, gastroenteral, vaginal, rectal, tracheal, urethral or pulmonary administration.

The therapeutic uses of the present medications or the method of treatments using the same are enabled by the delivery of efficacious amounts of the active agent(s) carried by the nano-elements. Such agents whether WITC-miscible/polar-carrier-insoluble, so as to be contained or entrapped within the core of the nano-elements, or carrier-soluble, so as to form a shell of core-shell nano-elements can be analgesics, anesthetics, anti-addiction agents, anti-bacterials, anti-convulsants, anti-dementia agents, anti-depressants, anti-emetics, anti-fungals, anti-gout agents, anti-inflammatory agents, anti-migraine agents, anti-myasthenic agents, anti-mycobacterials, anti-neoplastics, anti-obesity agents, anti-parasitics, anti-parkinson agents, anti-psychotics, anti-spasticity agents, anti-virals, anxiolytics, bipolar agents, blood glucose regulators, cardiovascular agents, central nervous system agents, contraceptives, dental and oral agents, gastrointestinal agents, genetic/enzyme/protein disorder agents, genitourinary agents, hormonal agents (including: adrenal, pituitary, prostaglandins, sex hormones and thyroid), hormone suppressant (including: adrenal, pituitary and thyroid), immunological agents, infertility agents, inflammatory bowel disease agents, metabolic bone disease agents, ophthalmic agents, otic agents, respiratory tract agents, sexual disorder agents, skeletal muscle relaxants, sleep disorder agents and dietary supplements (such as: vitamins electrolytes, minerals, and metals). The medications prepared using nano-elements including such active agents are often known by similar denominations (and vice versa), the method of treatment using the same having corresponding names readily appreciated that shall not be listed. For illustration, if the active agent is an analgesic agent, the medication can be considered an analgetic medicament that can be administered for the treatment of pain.

The present nano-elements may also be used for the delivery of diagnostic agents and the preparation of compositions that can be used for diagnostic purposes following their administration to a subject or object to be diagnosed therewith.

The present compositions may also be agrochemical compositions for the treatment of an object in need thereof by NSE administration thereto, such medications being generally in the form of solutions or emulsions, or in the form of powders, granules or pellets for preparing the same, to be administered to a suitable object, such as a plant, the administration being by injecting to the plant (e.g., to the trunk of a tree) or by drenching of the soil with the agrochemical composition.

The active agents that can be used in nano-elements for the preparation of such agrochemical products can be acaricides, algicides, an anthelmintic, an anti-moth, an avicide, a bactericide, a chemo-sterilant, a fertilizer, a fungicide, a herbicide, an insecticide including an ovicide, an insect repellent, an insect pheromone, a molluscicide, a nematicide, a nitrification inhibitor, a pesticide, a pest repellent, a plant growth promotor, a rodenticide, a termicide, a virucide, and a plant wound protectant.

Preparation of the aforesaid medications in the dosage forms suited for their mode of administration and their intended therapeutic activity, in the broad sense given herein to this term, can be conventionally conducted. Similarly, the treatment by administration of such medications can also be implemented in manners traditional for such treatments, and need not be detailed herein.

As previously described, the properties of the nano-elements may allow controlling the release profile of the active agent(s) from the nano-elements. Hence, in some embodiments, the medications used in the present invention allow the release of the active agent(s) over an extended period of time of at least twelve hours, whereby the medication can be considered a sustained-release medication with respect to said active agent(s).

### EXAMPLES

### Materials

The materials used in the following examples are listed in Table 1 below. The reported properties were retrieved from the product data sheets provided by the respective suppliers or estimated by standard methods. Unless otherwise stated, all materials were purchased at highest available purity level. N/A means that a particular information is not available.

TABLE 1

| Component | Chemical Name | Product Name | Supplier | CAS No. |
|---|---|---|---|---|
| Biodegradable | Polycaprolactone | PCL-14 | Sigma Aldrich ®, | 24980-41-4 |
| WITCs | | MW ~14 kDa | USA | |
| | | PCL-25 | Polysciences, | |
| | | MW ~25 kDa | USA | |
| | | PCL-37 | | |
| | | MW ~37 kDa | | |
| | | PCL-45 | Sigma-Aldrich ®, | |
| | | MW ~45 kDa | USA | |
| | | PCL-80 | Sigma-Aldrich ®, | |
| | | MW ~80 kDa | USA; | |
| | | | Polysciences, | |
| | | | USA | |
| | Polylactic acid | Ecosoft ® 608XF | Micro Powders, | 9051-89-2 |
| | | | USA | |
| | Poly(lactic-co- | PLGA | Nomisma | 26780-50-7 |
| | glycolic acid) | MW ~24-38 kDa | Healthcare | |
| | | | Private, India | |
| | Poly(butylene | PBSA | Shaanxi Dideu | 67423-06-7 |
| | succinate-co- | | Medichem, | |
| | adipate) | | China | |
| | Coenzyme Q10 | Coenzyme Q10 | Thermo Fisher | 303-98-0 |
| | | | Scientific, USA | |
| | Dewaxed shellac | Dewaxed shellac | Worlée, | 9000-59-3 |
| | | type 4621 | Germany | |
| | Gum rosin | Gum rosin | Guangxi | 8050-09-7 |
| | | | Wuzhou Foreign | |
| | | | Trade, China | |
| Non- | Poly(ethylene-co- | Nucrel ™ 599 | DuPont, USA | 25053-53-6 |
| biodegradable | methacrylic acid) | (PEMAA) | | |
| WITCs | Poly(ethylene-co- | Primacor ® 5990i | Dow, USA | 9010-77-9 |
| | acrylic acid) | (PEAA) | | |
| | Poly(ethylene-co- | Elvax ™ 250 | Dow, USA | 24937-78-8 |
| | vinyl acetate) | (PEVA) | | |
| SFAs | N,N-Dimethyl- | DMDA | Sigma Aldrich ®, | 112-18-5 |
| | dodecylamine | | USA | |
| | Cetrimonium | CTAC | Sigma Aldrich ® | 112-02-7 |
| | chloride | | USA | |
| | Oleyl amine | Oleyl amine | Arcos Organics, | 112-90-3 |
| | | | USA | |
| | Octyl amine | Octyl amine | Sigma Aldrich ®, | 111-86-4 |
| | | | USA | |
| | oleyl bis-(2- | Genamin ® O 020 | Clariant, | 25307-17-9 |
| | hydroxyethyl) | Special | Switzerland | |
| | amine | | | |
| | Caprylic acid | Caprylic acid | Y.S. Ashkenazi | 124-07-2 |
| | | | Agencies, Israel | |
| Non-volatile | Dibutyl adipate | Cetiol ® B | BASF ®, | 105-99-7 |
| liquids | | | Germany | |
| | C₁₂-C₁₅ alkyl | Pelemol ® 256 | Phoenix | 68411-27-8 |
| | benzoate | | Chemical, Inc., | |
| | | | USA | |
| | Dicaprylyl | Cetiol ® CC | BASF ®, | 1680-31-5 |
| | carbonate | | Germany | |
| | Triethyl O- | Citrofol ® All | Jungbunzkauer, | 77-89-4 |
| | acetylcitrate | | Switzerland | |
| | Dibutyl sebacate | Dibutyl sebacate | Sigma Aldrich ®, | 109-43-3 |
| | | | USA | |
| | Isoparaffin | Isopar ™ M | ExxonMobil, | 64742-47-8 |
| | | | USA | |
| | Benzyl alcohol | Benzyl alcohol | Ziv Chemicals, | 100-51-6 |
| | | | Israel | |
| Polar carriers | Glycerol | Glycerol | Sigma Aldrich ®, | 7325-17-9 |
| | | | USA | |
| | Propylene glycol | Propylene glycol | Chen Shmuel | 57-55-6 |
| | | | Chemicals, | |
| | | | Israel | |
| Surfactants | D-α-tocopherol | Vitamin E TPGS | Antares Health | 9002-96-4 |
| | polyethylene | | Products, USA | |
| | glycol 1000 | | | |
| | succinate | | | |
| (enulsifiers) | Poloxamer | Kolliphor ® P 407 | BASF ® | 691397-13-4 |
| | (poly(ethylene | | Germany | |
| | glycol)-block- | | | |
| | poly(propylene | | | |
| | glycol)-block- | | | |
| | poly(ethylene | | | |
| | glycol)) | | | |
| | Mixture of: | Tefose ® 63 | Gattefossé, | 9004-99-3 |
| | polyoxyl 6 | | France | 111-60-4 |
| | stearate type I, | | | |
| | ethylene glycol | | | |
| | stearates and | | | |
| | polyoxyl 32 | | | |
| | stearate type I | | | |
| | Mixture of: | Olivatis ® 12C | Medolla Iberia, | N/A |
| | olive oil | | Spain | |
| | polyglyceryl-6 | | | |
| | esters and | | | |
| | polyglyceryl-6 | | | |
| | pentaoleate | | | |
| | Olive oil PEG-8 | Olivatis ® 15C | | 103819-46-1 |
| | esters | | | |
| | Mixture of: | Olivatis ® 18C | | 85711-62-2 |
| | olive oil | | | 25383-99 |
| | polyglyceryl-6 | | | 67762-27-0 |
| | esters, | | | |
| | sodium stearoyl | | | |
| | lactylate and | | | |
| | cetearyl alcohol | | | |
| | Mixture of: | Olivatis ® 20C | | 85711-62-2 |
| | olive oil | | | 110615-47-9 |
| | polyglyceryl-6 | | | |
| | esters and | | | |
| | lauryl glucoside | | | |
| | Mixture of: | Olivatis ® 21C | | N/A |
| | olive oil PEG-6 | | | |
| | esters and | | | |
| | olive oil | | | |
| | polyglyceryl-6 | | | |
| | esters | | | |
| | Ascorbyl | Ascorbyl | Spec-Chem | 137-66-6 |
| | palmitate | palmitate | Industry, China | |
| | Polyglyceryl-10 | Nikkol Decaglyn | NIKKO | 86637-84-5 |
| | pentaoleate | 5-OV | Chemicals, | |
| | Polyglyceryl-10 | Nikkol Decaglyn | Japan | 126449-40-9 |
| | pentaisostearate | 5-ISV | | |
| | Oleoyl polyoxyl-6 | Labrafil ® M 1944 | Gattefossé, | 69071-70-1 |
| | glycerides | CS | France | |
| | Disodium laureth | Texapon ® SB 3 | BASF ®, | 68815-56-5 |
| | sulfosuccinate | KC | Germany | |
| | | Miconate LES(B) | Miwon Specialty | 39354-45-5 |
| | | | Chemicals, | |
| | Disodium lauryl | Miconate DLS(P) | Korea | 13192-12-6 |
| | sulfosuccinate | | | |
| | Mixture of: | Cola ®Det EQ-154 | Colonial | 26838-05-1 |
| | disodium lauryl | | Chemical, USA | 68439-57-6 |
| | sulfosuccinate, | | | 4292-10-8 |
| | sodium C₁₄-C₁₆ | | | |
| | olefin sulfonate | | | |
| | and | | | |
| | lauramidopropyl | | | |
| | betaine | | | |
| | Mixture of: | Olivoil ® glutamate | Kalichem, Italy | 67762-27-0 |
| | olive oil and | | | 8005-44-5 |
| | glutamic acid | | | 31566-31-1 |
| | Polysorbate 80 | Tween ® 80 | Sigma-Aldrich ® | 9005-65-6 |
| | | | USA | |
| | EO/PO Block | Pluronic ® F-127 | BASF ®, | 9003-11-6 |
| | Copolymer | | Germany | |
| Surfactants | Sodium dioctyl | Sodium dioctyl | Sigma-Aldrich ®, | 577-11-7 |
| (hydrotropes) | sulfosuccinate | sulfosuccinate | USA | |
| | | (AOT) | | |
| | Adenosine | Adenosine | Antai Fine | 56-65-5 |
| | triphosphate | triphosphate | Chemical | |
| | (ATP) | | Technology, | |
| | | | China | |
| | Ammonium | Ammonium | Stepan, USA | 26447-10-9 |
| | xylenesulfonate | xylenesulfonate | | |
| | Carbamide | Urea | Chen Shmuel | 57-13-6 |
| | | | Chemicals, | |
| | | | Israel | |
| pH-modifying | Acetic acid | Acetic acid | Chen Shmuel | 64-19-7 |
| agents | | (99.8%) | Chemicals, | |
| | | | Israel | |
| | Hydrochloric acid | HCl (1M) | Sigma Aldrich ®, | 7647-01-0 |
| | | | USA | |
| | Lactic acid | Lactic acid | Chen Shmuel | 79-33-4 |
| | | | Chemicals, | |
| | | | Israel | |
| | Ammonium | Ammonium | Sigma Aldrich ®, | 1336-21-6 |
| | hydroxide | hydroxide | USA | |
| Water-soluble | Sodium | Low molecular | Xi'an Lyphar | 9067-32-7 |
| active agents | hyaluronate | weight hyaluronic | Biotech, China | |
| | | acid (LMW HA, | | |
| | | MW 3 kDa) | | |
| | L-ascorbic acid | Vitamin C | Sigma Aldrich ®, | 50-81-7 |
| | | | USA | |
| | Collagen peptides | Verisol ® | Gelita, Germany | 92113-31-0 |
| | Elastin | Elastin | Koken, Japan | 9007-58-3 |
| WITC-miscible | Benzoyl peroxide | Benzoyl peroxide | Sigma Aldrich ®, | 94-36-0 |
| (carrier- | | | USA | |
| insoluble) | Tadalafil | Tadalafil | Teva | 171596-29-5 |
| active agents | | | Pharmaceutical | |
| | Funapide | Funapide | industries, Israel | 1259933-16-8 |
| | Retinyl palmitate | Retinyl palmitate | Xiamen | 79-81-2 |
| | | | Kingdomway | |
| | | | Vitamin, China; | |
| | | | Kingdomway | |
| | | | Nutrition | |
| | Bakuchiol | Sytenol ® A | Sytheon, USA | 10309-37-2 |
| | Curcumin | Curcumin | Jiaherb, China | 458-37-7 |
| | Neem oil | Neem oil | Manorama | 84696-25-3 |
| | | | Industries, India | |
| | Castor oil | Castor oil | Sigma Aldrich ®, | 8001-79-4 |
| | | | USA | |
| | DL-alpha- | DL-alpha- | BOC Sciences ®, | 10191-41-0 |
| | tocopherol | Tocopherol | USA | |
| Fluorescent | 9-(diethylamino)- | Nile red | GIHI Chemicals, | 7385-67-3 |
| marker | 5H-benzo[a] | fluorescent dye | China | |
| | phenoxazin-5-one | | | |

### Equipment

Conductivity meter: Eutech CON 700 by Thermo Fisher Scientific, USA

Cryo-TEM: Transmission Electron Microscope (TEM), Talos 200C by Thermo Fisher Scientific™, USA, with a Lacey grid

DSC: Differential Scanning calorimeter DSC Q2000 (TA Instruments, USA)

Oven: DFO-240, by MRC, Israel

Particle Size Analyzer (Dynamic Light Scattering): Zen 3600 Zetasizer and Zetasizer Nano ZS, (by Malvern Instruments®, United Kingdom)

Sonicator: VCX 750, by Sonics & Materials, USA

Thermo-rheometer: Thermo Scientific (Germany) Haake Mars III, with a C_{20/1}° spindle, a gap of 0.052 mm, and a shear rate of 10 sec⁻¹.

### I. Preparation of Core Nano-Elements

### Example 1-1: Screening of Non-Volatile Liquids Adapted to Plasticize a WITC

In the present study, various candidates for non-volatile liquids (also referred to as plasticizers or swelling agents) were tested for their suitability to plasticize a water-insoluble thermoplastic compound (WITC), and specifically, a water-insoluble thermoplastic polymer (WITP).

Each one of the various liquids was incubated for 1 hour at 80° C. at a weight per weight ratio of 1:1 with PCL having a molecular weight of 14 kDa (PCL-14), namely 2 g of a non-volatile liquid were added to 2 g of PCL-14 in a glass vial, and the sealed vials were placed in an oven, pre-heated to the plasticizing temperature. Following incubation, the contents of the vials were mixed by hand for about 30 seconds, until clear solutions were obtained. The samples of plasticized polymers were allowed to cool down overnight (i.e., at least 12 hours) at room temperature so as to solidify. None of the liquids so tested displayed leaching out of the plasticized PCL-14, suggesting that they might be used satisfactorily at even higher weight per weight ratio.

Solid samples were then transferred to a rheometer where their viscosity was measured as a function of temperature between 20° C. and 80° C. at a ramping up temperature of 10° C./min. A reference made of unplasticized PCL-14 was included in the study, this control displaying a viscosity gradually decreasing with raising temperature from about 2×10⁵ mPa·s (as measured at 50° C.) to about 2×10⁴ mPa·s (as measured at 80° C.). For comparison, unplasticized PCL having higher molecular weights, PCL-37, PCL-45 and PCL-80 to be later detailed, provided viscosities of up to about 6.2×10⁶ mPa·s, as measured at 50° C. within the range of ramping up temperatures.

In additional measurements of viscosities in this range of temperatures for samples similarly prepared at a 1:1 weight ratio, the following non-volatile liquids were found to decrease viscosity. In the case of PCL-14, all provided for a second viscosity of less than 10⁴ mPa·s (as measured at 50° C.) as compared to the first viscosity of 2×10⁵ mPa·s for this WITC, hence affording a decrease of at least 1.5 log. These non-volatile liquids included caprylic acid, dicaprylyl carbonate, C₁₂-C₁₅ alkyl benzoate, triethyl citrate, citronellol, cyclohexane-carboxylic acid, dibutyl adipate, hinokitiol, linalool, menthol, propylene carbonate, terpinol, tert-butyl acetate, and thymol, available for instance from Sigma-Aldrich, BASF®, or Phoenix Chemical.

Based on the above-screening results, a first pair of WITP and non-volatile liquid, namely a PCL having a molecular weight of about 14 kDa (PCL-14) and dibutyl adipate, was selected. Additional combinations of WITCs and non-volatile liquids were similarly tested and found adapted for the preparation of nano-elements that can be combined with active agents, as suited for pharmaceutical or agrochemical compositions for the preparation of the present medications.

### Example 2-1: Nano-Suspensions of Nano-Elements in an Aqueous Polar Phase

An aqueous solution containing a surfactant mixture (comprising an emulsifier and hydrotropes) was prepared as follows: 6.6 g of distilled water, 0.3 g of ammonium xylenesulfonate, 0.1 g of adenosine triphosphate and 1 g of vitamin E TPGS were placed in a 20 ml glass vial and sonicated for 10 minutes (at 40% power, operated in pulses of 7 seconds, followed by 1 second breaks), until a clear aqueous solution intended to serve as liquid polar phase for the nano-elements of WITC was obtained.

A WITC premix was prepared as follows: in a separate 20 ml glass vial, 3 g of PCL-14 having a native melting temperature of about 62° C. (as determined by DSC), were combined with 7 g of Cetiol® B, and the vial was placed in an oven at a temperature of 70° C.-80° C. for 1 hour until the PCL-14 was completely melted. The vial was then mixed by hand for about 30 seconds, until a clear, homogenized solution of 30 wt. % melted PCL plasticized by 70 wt. % Cetiol® B was obtained. The melting temperature of the plasticized polymer was then determined by DSC, and was found to be about 50° C., plasticizing with Cetiol® B having effectively reduced the Tm of the polymer by more than 10° C.

2 g of the WITC premix containing the melted solution of plasticized polymer were added to the vial containing the 8 g of aqueous solution including the surfactants, and sonicated for 20 minutes (as previously described), at a shearing temperature of about 70° C., whereby a nano-emulsion containing nano-droplets of liquid polymer in an aqueous solution was obtained.

This composition is reported in Table 2-I(A) as Composition 2.1. Additional compositions were prepared according to similar procedures, each composition containing different components in different amounts, and prepared under different conditions, as specified in Tables 2-I(A)-2-I(F). Sonication, when performed, was done as described above. The values reported in the table correspond to the concentration of each component in weight percent (wt. %) by total weight of the composition, except for the values in the WITC premix section, which correspond to the weight percentage of each component in that particular premix. The nano-emulsions so produced were allowed to passively cool down to room temperature for 1 hour, allowing the relative solidification of the nano-droplets and, when applicable, the formation of a nano-dispersion. Alternatively, the nano-droplets remained liquid in the nano-emulsions at room temperature. The size of the core nano-particles so produced was measured by Dynamic Light Scattering (DLS) on samples of the compositions, diluted to 1:100 in water, and the measured median diameter per volume (D_{V}50) and per number (D_{N}50), as well as polydispersity indices (PDI), are also presented in the tables below.

TABLE 2-I(A)

| | Composition | | | | | | | |
|---|---|---|---|---|---|---|---|---|
| Component | 2.1 | 2.2 | 2.3 | 2.4 | 2.5 | 2.6 | 2.7 | 2.8 |

WITC premix

| | | | | | | | | |
|---|---|---|---|---|---|---|---|---|
| PCL-14 | 30 | 30 | 30 | 20 | 20 | 20 | 20 | 20 |
| Cetiol ® B | 70 | 70 | 50 | 60 | 60 | 60 | 60 | 60 |
| Tefose ® 63 | | | 20 | | | | | |
| Olivatis ® 12C | | | | 20 | 20 | | | |
| Olivatis ® 18C | | | | | | 20 | | |
| Olivatis ® 20C | | | | | | | 20 | |
| Olivatis ® 21C | | | | | | | | 20 |

| | | |
|---|---|---|
| Premix | 1 hr @80° C. | 2 min @80° C. by sonication |
| preparation | in oven | |

Nano-dispersion

| PCL-14 | 6 | 6 | 6 | 4 | 4 | 4 | 4 | 4 |
|---|---|---|---|---|---|---|---|---|
| Cetiol ® B | 14 | 14 | 10 | 12 | 12 | 12 | 12 | 12 |
| Vitamin E TPGS | 10 | 15 | | | | | | |
| Ammonium | 3 | 3.6 | | | | | | |
| xylenesulfonate | | | | | | | | |
| ATP | 1 | | | | | | | |
| Tefose ® 63 | | | 4 | | | | | |
| Sodium dioctyl | | | 4 | 5 | | 5 | 5 | 5 |
| sulfosuccinate | | | | | | | | |
| Olivatis ® 12C | | | | 4 | 4 | | | |
| Olivatis ® 15C | | | | | 20 | | | |
| Olivatis ® 18C | | | | | | 4 | | |
| Olivatis ® 20C | | | | | | | 4 | |
| Olivatis ® 21C | | | | | | | | 4 |
| Propylene glycol | | 10 | | | | | | |
| Water | 66 | 51.4 | 76 | 75 | 60 | 75 | 75 | 75 |
| Nano-sizing | 20 min @70° C. | | 1 min @80° C. | | | | | |
| D_{V}50 [nm] | 125.0 | 39.0 | 44.3 | 125.0 | 109.0 | 108.0 | 130.0 | 136.0 |
| D_{N}50 [nm] | 54.0 | 54.1 | 23.5 | 51.5 | 68.1 | 59.0 | 43.2 | 62.3 |
| PDI | 0.403 | 0.196 | 0.340 | N/A | 0.259 | N/A | 0.309 | N/A |

TABLE 2-I(B)

| | Composition | | | | | |
|---|---|---|---|---|---|---|
| Component | 2.9 | 2.10 | 2.11 | 2.12 | 2.13 | 2.14 |

WITC premix

| | | | | | | |
|---|---|---|---|---|---|---|
| PCL-14 | 20 | 30 | 30 | 20 | 20 | 20 |
| Cetiol ® B | 60 | 50 | 50 | 60 | 60 | 60 |
| Tefose ® 63 | | | | 20 | 20 | 20 |
| Ascorbyl | 20 | | | | | |
| plamitate | | | | | | |
| Nikkol Decaglyn | | 20 | | | | |
| 5-OV | | | | | | |
| Nikkol Decaglyn | | | 20 | | | |
| 5-ISV | | | | | | |

| | | |
|---|---|---|
| Premix | 2 min @80° C. by | 4 min @80° C. by sonication |
| preparation | sonication | |

Nano-dispersion

| PCL-14 | 4 | 6 | 6 | 4 | 4 | 4 |
|---|---|---|---|---|---|---|
| Cetiol ® B | 12 | 10 | 10 | 12 | 12 | 12 |
| Tefose ® 63 | | | | 4 | 4 | 4 |
| Sodium dioctyl | 20 | 4 | 4 | | | |
| sulfosuccinate | | | | | | |
| Ascorbyl | 4 | | | | | |
| plamitate | | | | | | |
| Nikkol Decaglyn | | 4 | | | | |
| 5-OV | | | | | | |
| Nikkol Decaglyn | | | 4 | | | |
| 5-ISV | | | | | | |
| Texapon ® SB 3 | | | | 5 | | |
| KC | | | | | | |
| Miconate DLS(P) | | | | | 5 | |
| Miconate LES(B) | | | | | | 5 |
| Olivatis ® 21C | | | | | | |
| Water | 60 | 76 | 76 | 75 | 75 | 75 |
| Nano-sizing | 1 min @80° C. | | | | | |
| D_{V}50 [nm] | 116.0 | 75.1 | 64.0 | 105.0 | 164.0 | 85.9 |
| D_{N}50 [nm] | 53.6 | 42.2 | 36.1 | 32.1 | 33.7 | 22.3 |
| PDI | 0.273 | 0.350 | 0.242 | 0.389 | 0.368 | 0.311 |

TABLE 2-I(C)

| | Composition | | | | | | | |
|---|---|---|---|---|---|---|---|---|
| Component | 2.15 | 2.16 | 2.18 | 2.19 | 2.20 | 2.21 | 2.22 | 2.23 |

WITC premix

| PCL-14 | 20 | 20 | 20 | 20 | 20 | 30 | 20 | 70 |
|---|---|---|---|---|---|---|---|---|
| Cetiol ® B | 60 | 60 | 60 | 60 | 60 | 40 | 60 | 30 |
| Tefose ® 63 | 10 | 20 | 20 | 20 | 13.3 | 20 | 13.3 | |
| Olivatis ® 21C | 10 | | | | | | | |
| Labrafil ® M | | | | | 6.7 | 10 | 6.7 | |
| 1944 CS | | | | | | | | |
| Premix | 1 min @80° C. by sonication | | | | | | | 4 min |
| preparation | | | | | | | | @80ºC by |
| | | | | | | | | sonication |

Nano-dispersion

| PCL-14 | 4 | 4 | 4 | 4 | 4 | 6 | 4 | 28 |
|---|---|---|---|---|---|---|---|---|
| Cetiol ® B | 12 | 12 | 12 | 12 | 12 | 8 | 12 | 12 |
| Tefose ® 63 | 2 | 4 | 4 | 4 | 2.7 | 4 | 2.7 | |
| Sodium dioctyl | 1 | 1.3 | | 1 | 1.3 | 1.3 | 1 | |
| sulfosuccinate | | | | | | | | |
| Olivatis ® 15C | 19 | 18.7 | | | | | | |
| Olivatis ® 21C | 2 | | | | | | | |
| Olivoil ® | | | 20 | 19 | 18.7 | 18.7 | 18 | |
| glutamate | | | | | | | | |
| Labrafil ® M | | | | | 1.3 | 2 | 1.3 | |
| 1944 CS | | | | | | | | |
| Urea | | | | | | | 1 | |
| Vitamin E TPGS | | | | | | | | 15 |
| Ammonium | | | | | | | | 25 |
| xylenesulfonate | | | | | | | | |
| Water | 60 | 60 | 60 | 60 | 60 | 60 | 60 | 20 |
| Nano-sizing | 1 min @80° C. | | | | | | | 2 min |
| | | | | | | | | @80ºC |
| D_{V}50 [nm] | 118.0 | 86.9 | 136.0 | 89.6 | 67.7 | 37.7 | 77.1 | N/A |
| D_{N}50 [nm] | 71.5 | 50.9 | 41.2 | 55.6 | 43.3 | 34.2 | 46.9 | 109 |
| PDI | 0.176 | 0.277 | 0.429 | 0.249 | 0.190 | 0.220 | 0.232 | N/A |

Additional compositions were similarly prepared, in which PCL-14 was replaced by various WITCs to form the core of the nano-elements. Compositions prepared using polycaprolactone of higher molecular weights, specifically, 25 kDa, 37 kDa, 45 kDa and 80 kDa, are reported in Table 2-I(D) as previously described. Compositions made using the natural WITCs, shellac and gum rosin as core constituents are reported in Table 2-I(E), as well as compositions prepared using the WITPs: poly(butylene succinate-co-adipate) (PBSA) and poly(lactic-co-glycolic acid) (PLGA), and the non-polymeric WITC, namely, Coenzyme Q10, the latter two prepared without any dedicated plasticizing non-volatile liquid. Notably, the polar carrier-insoluble active agent DL-alpha-tocopherol (vitamin E derivative), added to the WITC premix of compositions 2.31 and 2.32, may also serve as a plasticizing agent.

TABLE 2-I(D)

| | Composition | | | | | |
|---|---|---|---|---|---|---|
| Component | 2.24 | 2.25 | 2.26 | 2.27 | 2.28 | 2.29 |

WITC premix

| | | | | | | |
|---|---|---|---|---|---|---|
| PCL-25 | | 20 | 20 | | | |
| PCL-37 | | | | | 30 | |
| PCL-45 | 10 | | | | | |
| PCL-80 | | | | 20 | | 30 |
| Cetiol ® B | 70 | 60 | 60 | 60 | 40 | 40 |
| Tefose ® 63 | 20 | 20 | 20 | 20 | 20 | 20 |
| Labrafil ® M | | | | | 10 | 10 |
| 1944 CS | | | | | | |

| | | |
|---|---|---|
| Premix | 4 min | 1 min @80° C. by sonication |
| preparation | @80° C. by | |
| | sonication | |

Nano-dispersion

| PCL-25 | | 4 | 4 | | | |
|---|---|---|---|---|---|---|
| PCL-37 | | | | | 6 | |
| PCL-45 | 2 | | | | | |
| PCL-80 | | | | 4 | | 6 |
| Cetiol ® B | 14 | 12 | 12 | 12 | 8 | 8 |
| Tefose ® 63 | 4 | 4 | 4 | 4 | 4 | 4 |
| Sodium dioctyl | 5 | | 1.3 | | 1.3 | 1.3 |
| sulfosuccinate | | | | | | |
| Cola ®Det EQ- | | 5 | | | | |
| 154 | | | | | | |
| Olivatis ® 15C | | | 18.7 | | | |
| Olivoil ® | | | | 20 | 18.7 | 18.7 |
| glutamate | | | | | | |
| Labrafil ® M | | | | | 2 | 2 |
| 1944 CS | | | | | | |
| Water | 75 | 75 | 75 | 60 | 60 | 60 |
| Nano-sizing | 1 min @80° C. | | | | | |
| D_{V}50 [nm] | 157.0 | 106.0 | 124.0 | 74.2 | 35.1 | 64.8 |
| D_{N}50 [nm] | 34.5 | 33.6 | 101.0 | 35.8 | 18.2 | 49.1 |
| PDI | 0.262 | 0.371 | 0.048 | 0.239 | 0.214 | 0.156 |

TABLE 2-I(E)

| | Composition | | | | | | |
|---|---|---|---|---|---|---|---|
| Component | 2.30 | 2.31 | 2.32 | 2.33 | 2.34 | 2.35 | 2.36 |

WITC premix

| | | | | | | | |
|---|---|---|---|---|---|---|---|
| PLA | 10 | | | | | | |
| PBSA | | 5 | 5 | | | | |
| PLGA | | | | 6 | | | |
| Shellac | | | | | 20 | | |
| Gum rosin | | | | | | 10 | |
| Coenzyme | | | | | | | 50 |
| Q10 | | | | | | | |
| Cetiol ® B | 70 | | | | | 70 | |
| Benzyl alcohol | | | | | 80 | | |
| Tefose ® 63 | 20 | 13 | 13 | | | 13 | 50 |
| Labrafil ® M | | 7 | 7 | | | 7 | |
| 1944 CS | | | | | | | |
| DL-alpha- | | 75 | 75 | | | | |
| tocopherol | | | | | | | |
| Olivatis ® 21C | | | | 94 | | | |

| | | | | |
|---|---|---|---|---|
| Premix | 4 min | 2 min @80° C. by | 1 min @80° C. | 12 min |
| preparation | @80ºC by | sonication | by sonication | @70° C. |
| | sonication | | | in oven |

Nano-dispersion

| PLA | 1 | | | | | | |
|---|---|---|---|---|---|---|---|
| PBSA | | 0.5 | 0.5 | | | | |
| PLGA | | | | 0.6 | | | |
| Shellac | | | | | 2 | | |
| Gum rosin | | | | | | 1 | |
| Coenzyme | | | | | | | 2.5 |
| Q10 | | | | | | | |
| Cetiol ® B | 7 | | | | | 7 | |
| Benzyl alcohol | | | | | 8 | | |
| Tefose ® 63 | 2 | 1.3 | 1.3 | | | 1 | 2.5 |
| Sodium dioctyl | 5 | 1.3 | | 1.3 | 1 | 1.3 | |
| sulfosuccinate | | | | | | | |
| Cola ®Det | | | | | | | |
| EQ-154 | | | | | | | |
| Olivatis ® 15C | | | | | | | |
| Olivoil ® | | 18.7 | | 18.7 | | 18.7 | 20 |
| glutamate | | | | | | | |
| Labrafil ® M | | 0.7 | 0.7 | | | 1 | |
| 1944 CS | | | | | | | |
| Tween ® 80 | | | | | 10 | | |
| DL-alpha- | | 7.5 | 7.5 | | | | |
| tocopherol | | | | | | | |
| Pluronic ® | | | 5 | | | | |
| F-127 | | | | | | | |
| Olivatis ® 21C | | | | 9.4 | | | |
| Water | 85 | 70 | 85 | 70 | 79 | 70 | 75 |
| Nano-sizing | 1 min @80° C. | | | | | | |
| D_{V}50 [nm] | 121.0 | 68.1 | 72.9 | 49.2 | 104 | 71.3 | 61.3 |
| D_{N}50 [nm] | 38.2 | 46.1 | 51.3 | 40.5 | 89.1 | 60.5 | 45.9 |
| PDI | 0.224 | 0.155 | 0.325 | 0.114 | 0.022 | 0.086 | 0.595 |

More compositions were prepared using other non-volatile liquids instead of Cetiol® B, namely, Pelemol® 256, Cetiol® CC and dibutyl sebacate. These compositions are reported in Table 2-I(F), as previously described.

TABLE 2-I(F)

| | Composition | | |
|---|---|---|---|
| Component | 2.37 | 2.38 | 2.39 |

WITC premix

| PCL-14 | 10 | 10 | 30 |
|---|---|---|---|
| Pelemol ® 256 | 60 | | |
| Cetiol ® CC | | 60 | |
| Dibutyl sebacate | | | 40 |
| Olivatis ® 21C | 30 | | |
| Vitamin E TPGS | | 30 | |
| Tefose ® 63 | | | 20 |
| Labrafil ® M 1944 CS | | | 10 |
| Premix preparation | 1 min @ | | 2 min @ |
| | 80° C. | | 80° C. by |
| | by sonication | | sonication |
| Nano-dispersion | | | |
| PCL-14 | 1 | 1 | 6 |
| Pelemol ® 256 | 6 | | |
| Cetiol ® CC | | 6 | |
| Dibutyl sebacate | | | 8 |
| Olivatis ® 21C | 3 | | |
| Vitamin E TPGS | | 3 | |
| Tefose ® 63 | | | 4 |
| Labrafil ® M 1944 CS | | | 2 |
| Olivoil ® glutamate | 20 | 20 | |
| Sodium dioctyl | | | 2 |
| sulfosuccinate | | | |
| Water | 70 | 70 | 78 |
| Nano-sizing | 1 min @80° C. | | |
| D_{V}50 [nm] | 86.4 | 125.0 | 73.0 |
| D_{N}50 [nm] | 54.8 | 55.5 | 37.3 |
| PDI | 0.245 | 0.358 | 0.228 |

Compositions using non-biodegradable polymers for the formation of the cores of the nano-elements were also prepared. These compositions are reported in Table 2-I(G), as previously described.

TABLE 2-I(G)

| | Composition | |
|---|---|---|
| Component | 2.40 | 2.41 |

WITC premix

| | | |
|---|---|---|
| PEAA | 10 | |
| PEMMA | | 10 |
| Cetiol ® CC | 70 | 70 |
| Tefose ® 63 | 13 | 13 |
| Labrafil ® M 1944 CS | 7 | 7 |

| | |
|---|---|
| Premix preparation | 2 min @80° C. by sonication |

Nano-dispersion

| PEAA | 1 | |
|---|---|---|
| PEMMA | | 1 |
| Cetiol ® CC | 7 | 7 |
| Tefose ® 63 | 1.3 | 1.3 |
| Labrafil ® M 1944 CS | 0.7 | 0.7 |
| Olivoil ® glutamate | 18.7 | 18.7 |
| Sodium dioctyl | 1.3 | 1.3 |
| sulfosuccinate | | |
| Water | 70 | 70 |
| Nano-sizing | 1 min @80° C. | |
| D_{V}50 [nm] | 41.6 | 83.1 |
| D_{N}50 [nm] | 27.2 | 64.3 |
| PDI | 0.202 | 0.232 |

As can be seen in Tables 2-I(A)-2-I(G), the present method is suitable to prepare nano-suspensions of core nano-elements containing a WITC within their cores, the nano-elements having Dv50 and D_{N}50 not exceeding 200 nm, these values being even lower than 100 nm for some of the compositions above reported. The PDI of the populations of core nano-particles was at most about 0.4.

Samples corresponding to the above-described premixes were additionally tested for their viscosity at the end of the mixing step, when the WITCs were at least homogeneously blended with the polar-carrier-insoluble materials, and in most case plasticized by the non-volatile liquids if present. Viscosity was determined as previously described at a shear rate of 10 sec⁻¹ over a range of temperatures between 20° C. and 80° C., and for all samples so tested the viscosity as measured at 50° C. was typically found to be of less than 10⁶ mPa·s, being generally between 10³ mPa·s and 10⁵ mPa·s, often not exceeding 5×10⁴ mPa·s, and many samples even having a viscosity of less 10⁴ mPa·s.

### Example 3-1: Nano-Suspensions of Nano-Elements Including Polar-Carrier-Insoluble Active Agents in the WITC

In the present example, an active agent was added to the WITC. The water-insoluble active agents: retinol palmitate (MW ˜525 g/mol), tadalafil (MW ˜389 g/mol), funapide (MW ˜429 g/mol), neem oil (MW ˜720 g/mol) and castor oil (MW ˜927 g/mol) were used to exemplify the incorporation of a polar-carrier-insoluble and WITC-miscible active agents into the core of nano-elements including the WITC. Retinol palmitate can be considered as an exemplary vitamin, as may be suited for the preparation of dietary supplements or nutraceuticals; tadalafil can be considered as an exemplary active agent for the treatment of erectile dysfunction, pulmonary hypertension and benign prostate enlargement; and funapide can be considered as an exemplary analgesic for the treatment of a variety of chronic pain conditions. While neem oil and castor oil can be used for living subjects (e.g., respectively to reduce blood sugar levels, or as a laxative), they can also illustrate exemplary pesticides suitable for the systemic treatment of plants.

A WITC/active agent premix was prepared in a 20 ml glass vial by combining 2 g of PCL-14, 1 g of retinol palmitate, 1 g of Olivatis® 12C as a surfactant and 6 g of Cetiol® B as a plasticizing non-volatile liquid. The vial was sonicated for 2 minutes (as previously described) at a temperature of about 80° C., to obtain a clear homogenized solution of plasticized WITC. The WITC/active agent premix was maintained in an oven at a temperature of 80° C. until mixed with the aqueous phase.

In a separate 20 ml glass vial, 7.5 g of distilled water and 0.5 g of sodium dioctyl sulfosuccinate, as an additional surfactant being a hydrotrope, were placed and sonicated for 1 minute at a temperature of about 60° C., until a clear aqueous solution intended to serve as liquid polar phase was obtained.

2 g of the hot WITC/active agent premix were then added to the vial containing the 8 g of aqueous solution and sonicated for 1 minute at a shearing temperature of about 70-80° C., whereby a nano-emulsion containing nano-droplets of liquid PCL including retinol palmitate dispersed in an aqueous polar phase was obtained.

This composition is reported in Table 3-I as Composition 3.1. Other compositions were prepared according to similar procedures, containing different components in different amounts, as specified in the table. Tadalafil and funapide were each first combined with the non-volatile liquid and sonicated for 2 minutes at 80° C. to obtain a clear solution containing the active agents, and then combined with the WITC and other components as described above.

The values reported in the table correspond to the concentration of each component in weight percent (wt. %) by total weight of the composition, except for the values in the WITC/active agent premix section, which correspond to the weight percentage of each component in that particular premix. The nano-emulsions so produced were allowed to passively cool down to room temperature for 1 hour, allowing the relative solidification of the nano-droplets. The extent of solidification so that at room temperature the nano-droplets are sufficiently solid to constitute a nano-dispersion or conversely sufficiently liquid to constitute a nano-emulsion can be assessed by sampling the pre-mix for a volume of the mixed materials, the isolated samples being similarly allowed to cool down without having been sheared into a liquid phase. The size and PDI values of the core nano-particles so produced, measured by DLS as previously described, are also presented in Table 3-1.

TABLE 3-1

| Composition | | | | | | | | |
|---|---|---|---|---|---|---|---|---|
| 3.1 | 3.2 | 3.3 | 3.4 | 3.5 | 3.6 | 3.7 | 3.8 | 3.9 |

WITC/active agent premix

| | | | | | | | | | |
|---|---|---|---|---|---|---|---|---|---|
| PCL | 20 | 33 | 20 | 20 | 20 | 20 | 20 | 20 | 20 |
| Cetiol ® B | 60 | 47 | 60 | 60 | 59.4 | 57 | 58 | 58 | 60 |
| Tefose ® 63 | | 13.45 | | | 13 | 13 | 13 | 13 | 10 |
| Olivatis ® 12C | | | | 10 | | | | | |
| Olivatis ® 21C | | | | | | | | | 5 |
| Labrafil ® M | | | | | 7 | 7 | 7 | 7 | |
| 1944 CS | | | | | | | | | |
| Retinol | 20 | 6.55 | 20 | 10 | | | | | 5 |
| palmitate | | | | | | | | | |
| Tadalafil | | | | | 0.6 | | | | |
| Funapide | | | | | | 3 | | | |
| Neem oil | | | | | | | 2 | | |
| Castor oil | | | | | | | | 2 | |

| | | |
|---|---|---|
| Premix | 2 min @80° C. by sonication | 1 min @80° C. |
| preparation | | by sonication |

Nano-dispersion

| PCL | 6 | 9.9 | 4 | 4 | 4 | 4 | 2 | 2 | 4 |
|---|---|---|---|---|---|---|---|---|---|
| Cetiol ® B | 18 | 14.1 | 12 | 12 | 11.9 | 11.4 | 5.8 | 5.8 | 12 |
| Vitamin E | 10 | | | | | | | | |
| TPGS | | | | | | | | | |
| Ammonium | 20 | | | | | | | | |
| xylenesulfonate | | | | | | | | | |
| Tefose ® 63 | | 4 | | | 2.6 | 2.6 | 1.3 | 1.3 | 2 |
| Sodium dioctyl | | 5 | 5 | 5 | 1.3 | 1.3 | 1.3 | 1.3 | 2 |
| sulfosuccinate | | | | | | | | | |
| Olivatis ® 12C | | | | 2 | | | | | |
| Olivatis ® 15C | | | | | | | | | 18 |
| Olivatis ® 20C | | | | | | | | | 1 |
| Labrafil ® M | | | | | 1.4 | 1.4 | 0.7 | 0.7 | |
| 1944 CS | | | | | | | | | |
| Olivoil ® | | | | | 18.7 | 18.7 | 18.7 | 18.7 | |
| glutamate | | | | | | | | | |
| Retinol | 6 | 2 | 4 | 2 | | | | | 1 |
| palmitate | | | | | | | | | |
| Tadalafil | | | | | 0.1 | | | | |
| Funapide | | | | | | 0.6 | | | |
| Neem oil | | | | | | | 0.2 | | |
| Castor oil | | | | | | | | 0.2 | |
| Water | 40 | 65 | 75 | 75 | 60 | 60 | 70 | 70 | 60 |
| Nano-sizing | 2 min | 1 min @80° C. | | | | | | | |
| | @80ºC | | | | | | | | |
| D_{V}50 [nm] | 171.0 | 58.6 | 138.0 | 82.1 | 78.3 | 88.6 | 32.1 | 46.4 | 96.3 |
| D_{N}50 [nm] | 78.5 | 21.0 | 68.9 | 34.1 | 49.2 | 52.3 | 25.8 | 31.1 | 60.4 |
| PDI | 0.270 | 0.179 | 0.259 | 0.314 | 0.174 | 0.170 | 0.408 | 0.238 | 0.184 |

As can be seen in Table 3-1, the present method is suitable to prepare nano-suspensions of nano-elements containing in their cores a WITC and a carrier-insoluble active agent, the nano-elements having Dv50 and D_{N}50 not exceeding 200 nm, these values being even lower than 100 nm for some of the compositions above reported. The PDI of the populations of core nano-particles was at most about 0.3.

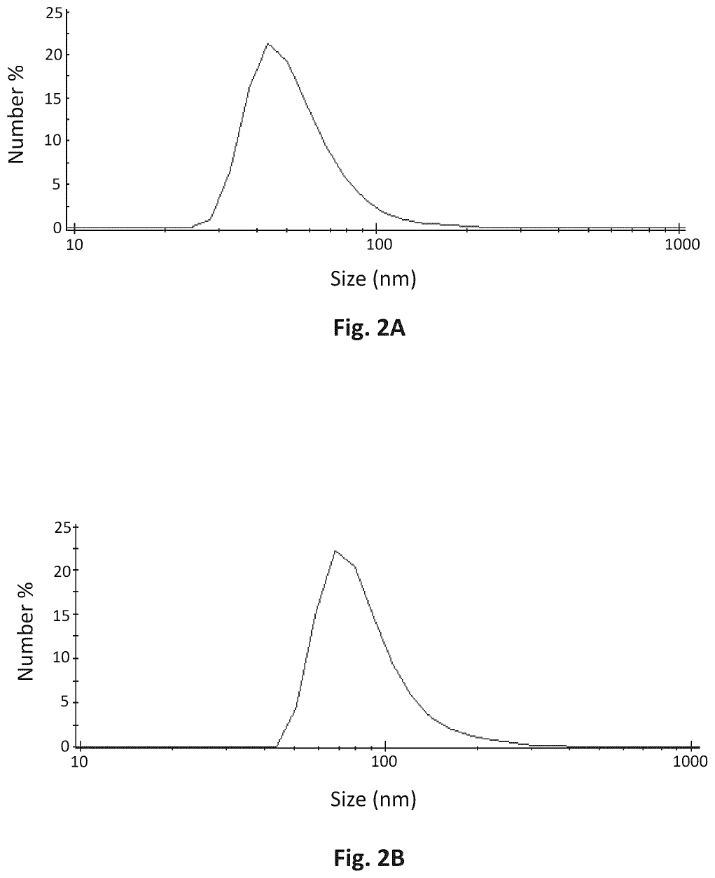

Representative results of particle size distribution in a sample of Composition 3.6, showing the percentage (per volume) of core nano-particles having hydrodynamic diameters in the range of 10-1,000 nm, are presented in FIG. 2A.

### Example 4-1: Nano-suspensions of WITC in a Liquid Polar Phase Including a Polar-Carrier-Soluble Active Agent

An aqueous solution containing a surfactant mixture (comprising an emulsifier and a hydrotrope) was prepared as follows: 4.4 g of distilled water, 0.6 g of ammonium xylenesulfonate and 1 g of vitamin E TPGS were placed in a 20 ml glass vial and sonicated for 10 minutes (as previously described), until a clear aqueous solution including the surfactants and intended to serve as liquid polar phase was obtained.

In a separate 20 ml glass vial, a WITC premix was prepared as follows: 3 g of PCL-14 and 7 g of Cetiol® B were combined, and the vial was placed in an oven at a temperature of 80° C. for 1 hour until the PCL was plasticized and completely melted. The vial was then mixed by hand for about 30 seconds, until a clear, homogenized solution of 30 wt. % melted PCL, plasticized with 70 wt. % Cetiol® B was obtained.

2 g of the melted solution of plasticized polymer were added to the vial containing 6 g of the aqueous solution with the surfactants and sonicated for 20 minutes (as described above) at a shearing temperature of about 70° C., whereby a nano-emulsion containing nano-droplets of liquid polymer in an aqueous polar phase was obtained.

The nano-emulsion was allowed to passively cool down to room temperature for a cooling period of 1 hour, at which time 1 g of propylene glycol was added, and the contents of the vial were mixed by hand for 10 seconds. Subsequently, 1 g of LMW hyaluronic acid, as the polar-carrier-soluble active agent, was added, and the vial contents were again mixed by hand for about 10 seconds until complete dissolution of the HA in the liquid polar phase.

This composition is reported in Table 4-1 as Composition 4.1. Additional compositions were similarly prepared, containing different components in different amounts. The values reported in the table correspond to the concentration of each ingredient in weight percent (wt. %) by total weight of the composition, except for the values in the WITC premix section, which correspond to the weight percentage of each component in that particular premix. The size and PDI values of the nano-particles so produced, as measured by DLS as previously described, are also presented in Table 4-1.

TABLE 4-1

| | Composition | | |
|---|---|---|---|
| Component | 4.1 | 4.2 | 4.3 |

WITC premix

| | | | |
|---|---|---|---|
| PCL-14 | 30 | 30 | 30 |
| Cetiol ® B | 70 | 70 | 40 |
| Tefose ® 63 | | | 20 |
| Labrafil ® M 1944 CS | | | 10 |

| | | |
|---|---|---|
| Premix preparation | 10 min @ | 5 min @ |
| | 80° C. by | 80° C. by |
| | sonication | sonication |

Nano-dispersion

| PCL-14 | 6 | 5 | 6 |
|---|---|---|---|
| Cetiol ® B | 14 | 11.7 | 8 |
| Vitamin E TPGS | 10 | | |
| Kolliphor ® P 407 | | 8.3 | |
| Ammonium | 6 | 16.7 | |
| xylenesulfonate | | | |
| ATP | | 8.3 | |
| Tefose ® 63 | | | 4 |
| Labrafil ® M 1944 CS | | | 2 |
| Sodium dioctyl | | | 1.3 |
| sulfosuccinate | | | |
| Olivoil ® glutamate | | | 18.7 |
| Propylene glycol | 10 | 4.2 | |
| Water | 44 | 37.5 | |
| Nano-sizing | 20 min @ | | 1 min @ |
| | 70° C. by | | 80° C. by |
| | sonication | | sonication |
| Cooling period | 1 hour | | 12 hours |
| LMW HA | 10 | 8.3 | 0.1 |
| D_{V}50 [nm] | 56.3 | 141.0 | 74.9 |
| D_{N}50 [nm] | 41.0 | 96.7 | 52.3 |
| PDI | 0.195 | 0.212 | 0.210 |

As can be seen in Table 4-1, the present method is suitable to prepare nano-suspensions of nano-elements containing a WITC and a carrier-soluble active agent, the nano-elements having Dv50 and D_{N}50 not exceeding 200 nm, the PDI of the populations of nano-particles being of at most about 0.2.

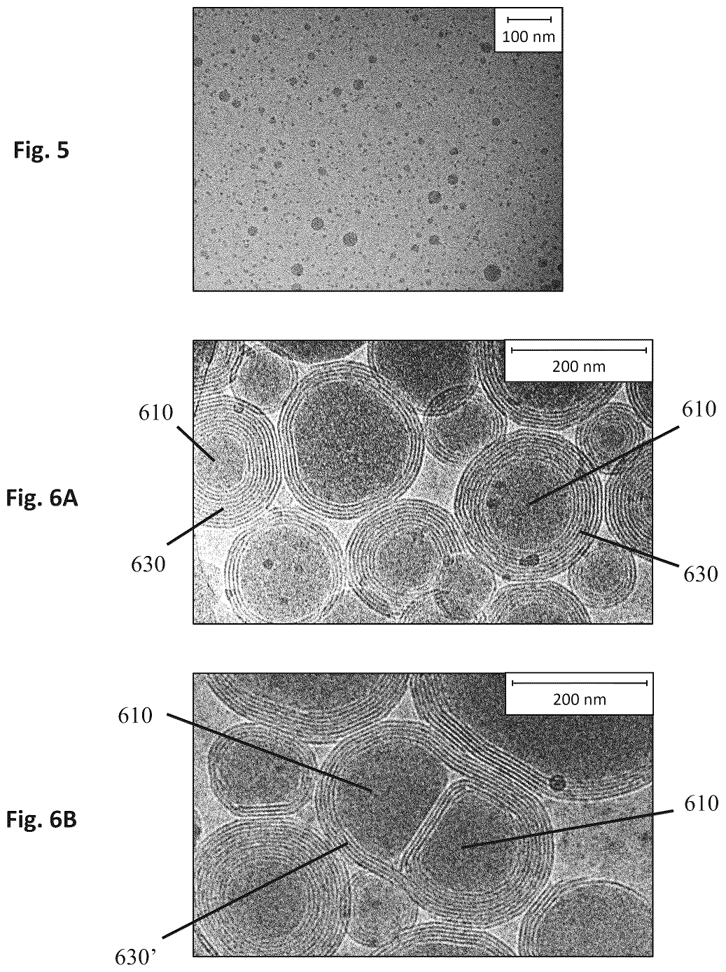

The size of the core nano-particles of Composition 4.1 was confirmed by microscopic TEM measurement of an image taken on a cryogenic cut of the nano-dispersion, the frozen nano-particles observed in the image having sizes in agreement with the measurements obtained by DLS. An exemplary image is shown in FIG. 5 where the nano-particles appear on the background as darker greyish globules, demonstrating the cores.

It is believed that the methods of Examples 3-I and 4-I can be combined, when a nano-dispersion is to include nano-elements of WITC(s), the dispersion including two types of active agents, the carrier-insoluble ones being disposed within the WITC(s) matrix in the nano-element and the carrier-soluble ones being disposed in the surrounding polar carrier.

### II. Preparation of Core-Shell Nano-Elements

### Example 1-11: Reducing Viscosity of the WITC

Further to the study previously performed in Example 1-I, where various non-volatile liquids were screened for their ability to plasticize WITCs, specifically PCL-14, in the present study, the viscosity of PCL-14 (referred to as PCL in the present example) and of its mixtures with a shell-forming agent, in addition to non-volatile liquids, used as plasticizing agents, were measured at different weight per weight ratios of the PCL to the plasticizing agents.

In a first series of experiments, the viscosity was measured on blends of materials forming a homogeneous mass adapted for nano-sizing by the present methods. PCL and a non-volatile liquid or a fatty amine serving as SFA at various weight per weight ratios were combined in a glass vial and the sealed vials were placed in an oven pre-heated to 80° C. The PCL: non-volatile liquid ratios tested were 1:1 and 1:2.33 and the PCL: fatty amine ratios tested were 1:0.1, 1:0.33, 1:0.5 and 1:1. Following 1 hour of incubation under heat, the contents of the vials were mixed by hand for about 30 seconds, until clear solutions were obtained. The samples were allowed to cool down overnight (i.e., at least 12 hours) at room temperature so as to solidify. None of the plasticizing agents so tested displayed leaching out of the solidified polymer, suggesting that they might be used at even higher proportion with respect to the PCL.

Solid samples were then transferred to a rheometer where their respective viscosity was measured as a function of temperature between room temperature and 70° C. at a ramping up temperature of 10° C./min. The viscosities (or 2^{nd} viscosities) of the samples as measured at 50° C. and 70° C. are summarized in Table 2-II(A), including in the first row the first viscosities of a reference sample made of PCL alone, unplasticized, under same conditions.

TABLE 2-II(A)

| | PCL: | PCL 2^{nd} | PCL 2^{nd} |
|---|---|---|---|
| | plasticizing | viscosity | viscosity |
| Plasticizing | agent | at 50° C. | at 70° C. |
| agent | w/w ratio | [mPa · s] | [mPa · s] |
| None | Not Relevant | 1.7 × 10⁵ | 7.0 × 10⁴ |
| Cetiol ® B | 1:1 | 5.0 × 10³ | 2.2 × 10³ |
| | 1:2.33 | 215 | 164 |
| Oleyl | 1:0.33 | 1.9 × 10³ | 1.7 × 10³ |
| amine | 1:0.5 | 6.7 × 10³ | 4.4 × 10³ |
| Trioctyl | 1:1 | 544 | 279 |
| amine | | | |

As can be seen from the above table, at the temperatures of 50° C. and 70° C., all of the materials tested as plasticizing agents (either the non-volatile liquids dedicated to this purpose or the fatty amines inherently contributing to this effect) decreased the viscosity of the PCL to less than 10⁴ mPa·s at the weight per weight ratios tested. Different weight ratios, such as with lower relative amount of plasticizing agent or fatty amine to WITC (e.g., 1:0.1 of oleyl amine), may be less efficient, however suitable ratios and agents capable of plasticizing a material can be readily determined by routine experimentation, as herein demonstrated.

Based on the above results or similar experiments, ratios of PCL: plasticizing agents providing a dynamic viscosity of 1×10⁴ mPa·s or less, as measured at least at 50° C., (though higher viscosities are permissible) were selected for the preparation of core-shell nano-particles to be used in pharmaceutical or agrochemical compositions, as detailed in the following examples.

In a second series of experiments, the viscosity was measured at the other end of the process, that is to say on core-shell nano-elements isolated from compositions prepared as detailed in the following examples. Nano-particles only containing PCL-14 were used as reference and compared to nano-particles containing either 1 wt. % or 5 wt. % of Genamin® O 020 Special (GenA2 in the following table) as fatty amine blended with the PCL and to nano-particles including 30 wt. % of PCL-14, 30 wt. % of Gen A2 and additionally containing 40 wt. % of a non-volatile liquid, either Cetiol® B or dibutyl sebacate (DBS). Viscosity was measured as a function of temperature (in ° C.) as previously described but up to 85° C. and the results in mPa·s are presented in decreasing order of temperature in Table 2-II (B).

TABLE 2-II(B)

| | | | | 30% PCL- | 30% |
|---|---|---|---|---|---|
| | | 99% | 95% | 14 + 30% | PCL-14 + |
| | | PCL-14 + | PCL-14 + | GenA2 + | 30% |
| | 100% | 1% | 5% | 40% | GenA2 + |
| Temp. | PCL-14 | GenA2 | GenA2 | Cetiol ® B | 40% DBS |
| 85 | 1.24 × 10⁴ | 1.14 × 10⁴ | 9.58 × 103 | 1.37 × 10⁴ | 9.15 × 10⁴ |
| 80 | 1.44 × 10⁴ | 1.32 × 10⁴ | 1.10 × 10⁴ | 7.57 × 10⁴ | 9.35 × 10⁴ |
| 75 | 1.66 × 10⁴ | 1.53 × 10⁴ | 1.28 × 10⁴ | 8.49 × 10⁴ | 9.42 × 10⁴ |
| 70 | 1.92 × 10⁴ | 1.79 × 10⁴ | 1.49 × 10⁴ | 8.77 × 10⁴ | 9.48 × 10⁴ |
| 65 | 2.26 × 10⁴ | 2.10 × 10⁴ | 1.74 × 10⁴ | 9.29 × 10⁴ | 9.51 × 10⁴ |
| 60 | 2.67 × 10⁴ | 2.49 × 10⁴ | 2.06 × 10⁴ | 9.99 × 10⁴ | 9.79 × 10⁴ |
| 55 | 3.20 × 10⁴ | 2.98 × 10⁴ | 2.46 × 10⁴ | 1.02 × 10⁵ | 9.91 × 10⁴ |
| 50 | 3.89 × 10⁴ | 3.62 × 10⁴ | 2.97 × 10⁴ | 1.08 × 10⁵ | 1.03 × 10⁵ |
| 45 | 4.79 × 10⁴ | 4.45 × 10⁴ | 3.64 × 10⁴ | 8.97 × 10⁵ | 4.69 × 10⁵ |
| 40 | 6.43 × 10⁴ | 6.27 × 10⁴ | 4.67 × 10⁴ | 2.57 × 10⁶ | 1.49 × 10⁶ |
| 35 | 1.17 × 10⁹ | 6.79 × 10⁷ | 8.29 × 10⁶ | 4.63 × 10⁶ | 2.15 × 10⁶ |
| 30 | 1.33 × 10⁹ | 1.30 × 10⁹ | 1.24 × 10⁹ | 6.19 × 10⁶ | 2.48 × 10⁶ |
| 25 | 1.35 × 10⁹ | 1.34 × 10⁹ | 1.25 × 10⁹ | 7.69 × 10⁶ | 2.71 × 10⁶ |

As can be seen from the above table, while at temperatures between 30° C. and 85° C., the presence of even minute amounts of fatty amines (e.g., 1 wt. % GenA2) could be detected by way of their plasticizing effect on the WITC, at lower temperatures the detection of a plasticizing effect required a higher proportion of a plasticizing fatty amine to WITC. Interestingly, even low amounts of fatty amines capable of plasticization (e.g., 5 wt. % GenA2) provided for core-shell nano-elements having a viscosity of less than 1×10⁷ mPa·s at near body temperature measured at 35° C.

### Example 2-II: Preparation of Core-Shell Nano-Dispersions

2 g of PCL-14, 2 g of the SFA N,N-dimethyldodecylamine (DMDA, a fatty amine) and 6 g of the non-volatile liquid Cetiol® B, serving as a dedicated plasticizer, were placed in a 20 ml glass vial. The vial contents were sonicated for 2 minutes at about 70° C. until a clear WITC/SFA premix including the non-volatile liquid was obtained.

In a separate 20 ml glass vial, 8 g of glycerol were heated to about 70° C. using a sonicator, followed by addition of 2 g of the hot premix prepared above, and the composition was sonicated for 5 minutes whilst maintained at 70° C. to obtain a nano-emulsion. The nano-emulsion was then allowed to cool down to room temperature until a nano-dispersion containing core-shell nano-particles was obtained.

This nano-dispersion (ND1) is reported in Table 3-II, which presents additional nano-dispersions prepared according to similar procedures, each nano-dispersion containing different components in different amounts, and prepared under different conditions, as specified in the table. Unless otherwise stated, the values reported in this and following tables describing compositions and their preparation, correspond to the concentration of each component in weight percent (wt. %) by total weight of the nano-dispersion, except for the values in the WITC/SFA premix section, which correspond to the weight percentage of each component in that particular premix which may further include any other WITC-miscible ingredient, such as a non-volatile liquid. Water refers to double distilled water.

The size of the nano-particles produced according to this or similar following examples was measured by Dynamic Light Scattering (DLS) on samples of the compositions, diluted to 1:100 in water, and the measured median diameter per number (D_{N}50), as well as polydispersity indices (PDI), are also presented in the relevant tables below. The maximum hydrodynamic diameters below which 10% and 90% of the sample number of particles exist (D_{N}10 and D_{N}90, respectively), when measured, are also presented in the pertinent tables.

TABLE 3-II

| | Composition | | | | | |
|---|---|---|---|---|---|---|
| Component | ND1 | ND2 | ND3 | ND4 | ND5 | ND6 |

WITC/SFA premix

| | | | | | | |
|---|---|---|---|---|---|---|
| PCL-14 | 20 | 30 | 20 | 20 | 20 | 20 |
| DMDA | 20 | | 20 | | | |
| CTAC | | 3 | | | | |
| Genamin ® O | | | | 20 | 20 | |
| 020 Special | | | | | | |
| Oleyl amine | | | | | | 20 |
| Cetiol ® B | 60 | 67 | 60 | 60 | 60 | 60 |

| | | |
|---|---|---|
| Premix | 2 min @70° C. | 1 min @80° C. |
| preparation | | |

Nano-dispersion

| PCL-14 | 4 | 6 | 2 | 4 | 4 | 8.9 |
|---|---|---|---|---|---|---|
| DMDA | 4 | | 2 | | | |
| CTAC | | 0.6 | | | | |
| Genamin ® O | | | | 4 | 4 | |
| 020 Special | | | | | | |
| Oleyl amine | | | | | | 8.9 |
| Cetiol ® B | 12 | 13.4 | 6 | 12 | 12 | |
| Citrofol ® All | | | | | | 26.7 |
| Olivoil ® | | | | 20 | | |
| glutamate | | | | | | |
| Sodium dioctyl | | | | | 1.7 | |
| sulfosuccinate | | | | | | |
| Glycerol | 80 | | 90 | | | |
| Propylene glycol | | | | | | 10.1 |
| Water | | 80 | | 60 | 78.3 | 45.4 |
| Nano-sizing | 5 min @70° C. | | | 1 min @80° C. | | |
| D_{N}10 [nm] | 117.0 | 57.4 | 75.6 | N/A | N/A | N/A |
| D_{N}50 [nm] | 176.0 | 78.3 | 100.0 | 50.4 | 98.7 | N/A |
| D_{N}90 [nm] | N/A | 126.0 | 293.0 | N/A | N/A | N/A |
| PDI | 0.277 | 0.460 | 0.477 | 0.224 | 0.151 | N/A |

As can be seen in Table 3-II, the present method is suitable to prepare nano-suspensions of core-shell nano-elements containing a WITC and a SFA in their cores, the nano-elements having a D_{N}50 not exceeding 200 nm, this value being even lower than 100 nm for some of the nano-suspensions reported above. The PDI of the populations of the core-shell nano-elements was at most about 0.5.

### Example 3-11: Preparation of Positively Charged Core-Shell Nano-Particles by Acid-Doping

8 g of double distilled water were placed in a 20 ml glass vial. 2 g of the nano-dispersion ND1 obtained in Example 2-II were added, and the contents of the vial were shaken by hand for about 10 seconds until a homogeneous mixture was obtained (referred to as charged ND1 or cND1). Zeta potential of the mixture was measured by Malvern Zetasizer Nano ZS and found to be −14.7 mV. Unless otherwise stated, all measurements made with this instrument (e.g., zeta potential and particle size distribution) were performed on samples diluted 1:100 in double distilled water. All pH measurements reported in this and following examples were made at least using a pH test strip adapted to the pH range of relevance, and confirmed in some cases by using a suitable pH meter.

1 drop of acetic acid was then added to the mixture, yielding a pH of 5.5, and the vial was shaken by hand for about 10 seconds until a nano-dispersion containing positively charged core-shell PCL/DMDA nano-particles was obtained, referred to as cND1′. The zeta potential of the nano-particles in the acid doped nano-dispersion was measured and confirmed to be positive with a charge of +52.4 mV.

The hydrodynamic diameter of the nano-particles obtained in the acid-doped cND1′ was determined by DLS and the D_{N}50 of the sample was found to be similar to that of the nano-particles of ND1, indicated in Table 3-II.

### Example 4-11: Preparation of Positively Charged Core-Shell Nano-Particles, in Absence of Acid

9.9 g of double distilled water were placed in a 20 ml glass vial. 0.1 g of nano-dispersion ND3 obtained in Example 2-II were added, and the contents of the vial were shaken by hand for 10 seconds until a homogeneous mixture, referred to as cND3, was obtained. The zeta potential of the mixture was measured and found to be +43 mV. The particle size distribution of the nano-particles so obtained was determined by DLS and the sample was found to have a D_{N}50 similar to that of the nano-particles of ND3, indicated in Table 3-II.

### Example 5-II: Preparation of Positively Charged Core-Shell Nano-Particles by Acid-Doping of Diluted Sample

9 g of double distilled water were placed in a 20 ml glass vial. 1 g of the nano-dispersion cND3, obtained in Example 4-II were added, and the contents of the vial were shaken by hand for 10 seconds until a homogeneous mixture, referred to as cND3′, was obtained. The zeta potential of the mixture was measured and found to be −9.9 mV. This mixture differs from the one described in Example 4-II by containing 10-times less ND3 in a same total weight of aqueous carrier. The 10-fold diluted nano-dispersion presently prepared displayed a relatively lower charging, having a charge of-9.9 mV, as compared to a charge of +43 mV in previous case.

1 drop of acetic acid was then added to the relatively diluted mixture, yielding a pH of 5.5, and the vial was shaken by hand for about 10 seconds until an acid-doped nano-dispersion containing positively charged core-shell PCL/DMDA nano-particles, referred to as cND3∝1, was obtained. The zeta potential of the acid-doped nano-dispersion cND3″ was measured and confirmed to be positive with a charge of ±7.8 mV. The hydrodynamic diameter of the obtained nano-particles was determined and the D_{N}50 was found to be similar to that of the nano-particles of ND3, indicated in Table 3-II.

Example 6-II: Preparation of core-shell nano-particles in a pH-modified polar carrier

2 g of PCL-14, 2 g of DMDA and 6 g of Cetiol® B were placed in a 20 ml glass vial. The vial contents were sonicated for 2 minutes at about 70° C. until a clear WITC/SFA premix was obtained.

In a separate 20 ml glass vial, 8 g of glycerol and 1 g of a 3 wt. % HCl solution (as the pH-modifying agent) were mixed, whereby a solution having a pH of 3 was obtained. 1 g of the hot WITC/SFA premix prepared above was added to the acidic liquid carrier, and the contents of the vial were sonicated for 30 seconds to obtain a nano-emulsion. The nano-emulsion was allowed to cool down to room temperature until a nano-dispersion was obtained.

The zeta potential of the nano-dispersion was measured and was found to be +72.8 mV, this charge resulting from the dilution of the sample in water. Hence, positively charged nano-particles were readily obtained following their sonication in the acidic polar carrier, instead of a two-step process, as previously described.

The nano-dispersion so obtained (ND7) is reported in Table 4-II(A). Additional nano-dispersions were prepared according to similar procedures, all being chargeable in the presence of water, each nano-dispersion containing different components in different amounts, and prepared under different conditions, as specified in Tables 4-II(A)-(C), parameters measured in samples of some of the nano-dispersions, diluted to 1:100 in double distilled water, and the resulting values being also presented in the tables below. The pH reported for the nano-dispersions ND15 and ND16 was obtained by adding about one drop of acetic acid to their respective aqueous phase.

TABLE 4-II(A)

| | Composition | | | | | | | | | |
|---|---|---|---|---|---|---|---|---|---|---|
| Component | ND7 | ND8 | ND9 | ND10 | ND11 | ND12 | ND13 | ND14 | ND15 | ND16 |

WITC/SFA premix

| | | | | | | | | | | |
|---|---|---|---|---|---|---|---|---|---|---|
| PCL-14 | 20 | 20 | 30 | 30 | 30 | 20 | 20 | 20 | | |
| PCL-37 | | | | | | | | | 20 | |
| PCL-80 | | | | | | | | | | 20 |
| DMDA | 20 | 20 | 12 | | | | | | | |
| Oleyl amine | | | | 25 | 25 | 20 | | | | |
| Genamin ® O | | | | | | | 20 | 20 | 20 | 20 |
| 020 Special | | | | | | | | | | |
| Cetiol ® B | 60 | 60 | 58 | | | | 60 | 60 | 60 | 60 |
| Citrofol ® All | | | | 45 | 45 | 60 | | | | |

| | | | | |
|---|---|---|---|---|
| Premix | 2 min @70° C. | 1 min | 1 min | 2 min @80° C. |
| preparation | | @70° C. | @80° C. | |

Nano-dispersion

| PCL-14 | 2 | 6 | 6 | 8.4 | 8.7 | 7.6 | 4 | 4 | | |
|---|---|---|---|---|---|---|---|---|---|---|
| PCL-37 | | | | | | | | | 4 | |
| PCL-80 | | | | | | | | | | 4 |
| DMDA | 2 | 6 | 2.4 | | | | | | | |
| Oleyl amine | | | | 7 | 7.2 | 7.6 | | | | |
| Genamin ® O | | | | | | | 4 | 4 | 4 | 4 |
| 020 Special | | | | | | | | | | |
| Cetiol ® B | 6 | 18 | 11.6 | | | | 12 | 12 | 12 | 12 |
| Citrofol ® All | | | | 12.6 | 13.1 | 22.8 | | | | |
| Glycerol | 80 | 60 | 70 | 37 | 20.3 | 17.7 | | | | |
| Propylene | | | | 10 | 14.5 | 12.7 | | | | |
| glycol | | | | | | | | | | |
| Water | 9.7 | 9.7 | 9.7 | 24.2 | 35.1 | 30.7 | 79.9 | 80 | 80 | 80 |
| HCl | 0.3 | 0.3 | 0.3 | 0.8 | 1.1 | 0.9 | | | | |
| Lactic acid | | | | | | | 0.1 | | | |
| Nano-sizing | 30 sec @70° C. | | | | | | 1 min @80° C. | | | |
| D_{N}10 [nm] | 55.3 | 77.4 | 95.4 | 58.3 | N/A | N/A | N/A | N/A | N/A | N/A |
| D_{N}50 [nm] | 73.5 | 106.0 | 138.0 | 80.3 | N/A | N/A | 73.4 | 75.2 | 96.1 | 41.9 |
| D_{N}90 [nm] | 106.0 | 186.0 | 238.0 | 134.0 | N/A | N/A | N/A | N/A | N/A | N/A |
| PDI | 0.424 | 0.332 | 0.148 | 0.242 | N/A | N/A | 0.202 | 0.240 | 0.227 | 0.355 |
| pH | 3 | 3 | 3 | 3 | 3 | 3 | 3 | 3.5 | 4 | 4 |
| Zeta potential | +72.8 | +73.0 | +59.7 | +48.9 | N/A | N/A | N/A | +56.0 | +68.4 | +85.0 |
| [mV] | | | | | | | | | | |

In addition to measuring the zeta potential of the nano-dispersions, the effect of the pH-modifying agent in the polar liquid carrier was monitored by measuring the pH changes along the preparation of the nano-dispersions. Taking ND7 for illustration, when combining an acidic glycerol solution having a pH of 3 with the WITC/SFA mixture, the SFA being the fatty amine DMDA, the pH increased to 6, indicating the elimination of the hydrogen ions of the liquid carrier, which are believed to have reacted with the amine groups of the fatty amine shell. The pH remained at 6 after cooling and increased to 6.5 following dilution with water, indicating that the amine groups remain protonated, as confirmed by the positive zeta potential.

Additional nano-dispersions prepared using various WITCs of synthetic and natural origins are summarized in Tables 4-II(B)-(C). The aqueous phases of the nano-dispersions reported in the table were pH-modified (by adding about one drop of acetic acid for ND17-ND23 and ND26, resulting in an acidic pH, and one drop of 2 drops of a 25% ammonium hydroxide solution to ND27, resulting in a basic pH. The amounts of the pH-modifying agent added to adjust the pH of ND24 and ND25 are specified in the table).

TABLE 4-II(B)

| | Composition | | | | | | |
|---|---|---|---|---|---|---|---|
| Component | ND17 | ND18 | ND19 | ND20 | ND21 | ND22 | ND23 |

WITC/SFA premix

| | | | | | | | |
|---|---|---|---|---|---|---|---|
| PLA | 20 | | | | | | |
| PLGA | | 20 | | | | | |
| PBSA | | | 20 | | | | |
| Coenzyme | | | | 20 | | | |
| Q10 | | | | | | | |
| Shellac | | | | | 20 | 20 | |
| Gum rosin | | | | | | | 20 |
| Genamin ® O | 20 | 20 | 20 | 20 | 80 | 20 | 20 |
| 020 Special | | | | | | | |
| Cetiol ® B | 60 | 60 | 60 | 60 | | 60 | 60 |

| | | | | | |
|---|---|---|---|---|---|
| Premix | 2 min @80ºC | 3 min @80° C. | 2 min @80° C. | 3 min @80ºC | 2 min @80ºC |
| preparation | | | | | |

Nano-dispersion

| PLA | 2 | | | | | | |
|---|---|---|---|---|---|---|---|
| PLGA | | 2 | | | | | |
| PBSA | | | 2 | | | | |
| Coenzyme | | | | 2 | | | |
| Q10 | | | | | | | |
| Shellac | | | | | 4 | 2 | |
| Gum rosin | | | | | | | 2 |
| Genamin ® O | 2 | 2 | 2 | 2 | 16 | 2 | 2 |
| 020 Special | | | | | | | |
| Cetiol ® B | 6 | 6 | 6 | 6 | | 6 | 6 |
| Water | 90 | 90 | 90 | 90 | 80 | 90 | 90 |
| Nano-sizing | 1 min @80° C. | | | | | | |
| D_{N}50 [nm] | 90.8 | 89.6 | 115.0 | 41.9 | 82.4 | 189.0 | 85.3 |
| PDI | 0.389 | 0.176 | 0.246 | 0.267 | 0.354 | 0.249 | 0.133 |
| pH | 4 | 4 | 4 | 4 | 3.5 | 4 | 4 |
| Zeta potential | +55.2 | +95.0 | +108.0 | +57.3 | +83.6 | +101.0 | +45.1 |
| [mV] | | | | | | | |

TABLE 4-II(C)

| | Composition | | | |
|---|---|---|---|---|
| Component | ND24 | ND25 | ND26 | ND27 |

WITC/SFA premix

| | | | | |
|---|---|---|---|---|
| PCL-14 | 30 | 30 | 20 | 20 |
| Genamin ® O | 30 | 30 | | |
| 020 Special | | | | |
| Caprylic acid | | | 20 | 20 |
| Cetiol ® B | 40 | | 60 | 60 |
| Dibutyl sebacate | | 40 | | |

| | |
|---|---|
| Premix preparation | 2 min @80° C. |

Nano-dispersion

| PCL-14 | 6 | 6 | 2 | 2 |
|---|---|---|---|---|
| Genamin ® O | 6 | 6 | | |
| 020 Special | | | | |
| Caprylic acid | | | 2 | 2 |
| Cetiol ® B | 8 | | 6 | 6 |
| Dibutyl sebacate | | 8 | | |
| Acetic acid | 1.5 | 0.25 | | |
| Water | 78.5 | 79.75 | 90 | 90 |
| Nano-sizing | 1 min @80° C. | | | |
| D_{N}50 [nm] | 47.9 | 56.8 | 84.5 | 37.0 |
| PDI | 0.210 | 0.169 | 0.500 | 0.463 |
| pH | 5 | 5 | 5.5 | 10 |
| Zeta potential [mV] | +48.2 | +68.7 | −36.4 | −55.1 |

The core-shell nano-elements of ND24 and ND25 were isolated and subjected to thermo-rheological analysis as reported in Table 2-II(B).

ND26 and ND27 contained a fatty acid as the SFA, promoting a negative charge, as indicated by the zeta potential. At a higher pH, the negative charge increased for ND27.

Nano-dispersions prepared using non-biodegradable thermoplastic polymers are summarized in Table 4-II(C). The pH reported for the nano-dispersions in the table was obtained by adding about one drop of acetic acid to their aqueous phase.

TABLE 4-II(C)

| | Composition | | |
|---|---|---|---|
| Component | ND28 | ND29 | ND30 |

Polymer/SFA premix

| | | | |
|---|---|---|---|
| EMAA | 20 | | |
| EAA | | 20 | |
| EVA | | | 10 |
| Genamin ® O | 20 | 20 | 20 |
| 020 Special | | | |
| Isopar ® M | 60 | 60 | 70 |
| Dibutyl sebacate | | | |

| | |
|---|---|
| Premix preparation | 3 min @80° C. |

Nano-dispersion

| EMAA | 2 | | |
|---|---|---|---|
| EAA | | 2 | |
| EVA | | | 1 |
| Genamin ® O 020 | 2 | 2 | 2 |
| Special | | | |
| Isopar ® M | 6 | 6 | 7 |
| Dibutyl sebacate | | | |
| Water | 90 | 90 | 90 |
| Nano-sizing | 1 min @80° C. | | |
| D_{N}50 [nm] | 79.5 | 101.0 | 173.0 |
| PDI | 0.378 | 0.256 | 0.129 |
| pH | 4 | 4 | 4 |
| Zeta potential [mV] | +81.7 | +106.0 | +80.4 |

### Example 7-II: Preparation of Core-Shell Nano-Particles Containing Acid in the Core

In the following example, the nano-dispersion was pH-modified from within the core of the core-shell, as opposed to forming the nano-emulsion by combining the core-shells with acidified polar carrier, as previously done (e.g., in example 6-II).

2 g of PCL-14, 2 g of Genamin® O 020 Special and 6 g of Cetiol® B were placed in a 20 ml glass vial. 0.01 g of anhydrous acetic acid were added, and the vial contents were sonicated for 2 minutes at about 70° C. until a clear WITC/SFA/acid premix was obtained.

In a separate 20 ml glass vial, 9 g of water were placed, 1 g of the hot WITC/SFA/acid premix prepared above was added, and the contents of the vial were sonicated for 1 minute at about 80° C. to obtain a nano-emulsion. The nano-emulsion was allowed to cool down to room temperature until a nano-dispersion was obtained.

The pH was measured and found to be 5. The zeta potential, was measured in samples of the nano-dispersion, diluted to 1:100 in double distilled water and was found to be +54.1 mV, indicating the positive charging of the nano-particles, supposedly due to the acetic acid leaching out of the core of the nano-particles into the polar carrier, thus protonating the amine heads in the shell of the nano-particles. D_{N}50 and PDI were similarly measured, and were found to be 35.3 nm and 0.198, respectively.

### Example 8-II: Preparation of Core-Shell Nano-Particles Containing WITC-Miscible Active Agents Within Their Cores

2 g of PCL-14 and 0.1 g of benzoyl peroxide as a WITC-miscible active agent were placed in a 20 ml glass vial. 2 g of Genamin® O 020 Special and 5.9 g of Cetiol® B were added, and the vial contents were sonicated for 2 minutes at about 70° C. until a clear WITC/SFA/active agent premix was obtained.

In a separate 20 ml glass vial, 9 g of water were placed and acidified to a pH of 4 by adding a drop of acetic acid. 1 g of the hot WITC/SFA/active agent premix prepared above was added to the acidic liquid carrier, and the contents of the vial were sonicated for 1 minute at about 80° C. to obtain a nano-emulsion. The nano-emulsion was allowed to cool down to room temperature until a nano-dispersion was obtained.

The nano-dispersion so obtained (ND31) is reported in Table 5-II. Additional nano-dispersions were prepared according to similar procedures, all being chargeable in the presence of water, each nano-dispersion containing a different WITC-miscible and water-insoluble active agent (either a pharmaceutical agent or an agricultural agent), as specified in the table.

The zeta potential, D_{N}50 and PDI were measured in samples of some of the nano-dispersions, diluted to 1:100 in double distilled water, resulting in their charging, and the values are presented in Table 5-II.

TABLE 5-11

| | Composition | | | | | | | | |
|---|---|---|---|---|---|---|---|---|---|
| Component | ND31 | ND32 | ND33 | ND34 | ND35 | ND36 | ND37 | ND38 | ND39 |

WITC/SFA/active agent premix

| | | | | | | | | | |
|---|---|---|---|---|---|---|---|---|---|
| PCL-14 | 20 | 20 | 20 | 20 | 20 | 20 | 20 | 20 | 20 |
| Benzoyl | 1 | | | | | | | | |
| peroxide | | | | | | | | | |
| Tadalafil | | 1 | | | | | | | 2 |
| Retinyl | | | 1 | | | | | | |
| palmitate | | | | | | | | | |
| Bakuchiol | | | | 1 | | | | | |
| Curcumin | | | | | 1 | | | | |
| Neem oil | | | | | | 1 | 60 | | |
| Castor oil | | | | | | | | 60 | |
| Genamin ® | 20 | 20 | 20 | 20 | 20 | 20 | 20 | 20 | 20 |
| O 020 | | | | | | | | | |
| Special | | | | | | | | | |
| Cetiol ® B | 59 | 59 | 59 | 59 | 59 | 59 | | | |
| Benzoyl | | | | | | | | | 58 |
| benzoate | | | | | | | | | |

Nano-dispersion

| | | | | | | | | | |
|---|---|---|---|---|---|---|---|---|---|
| PCL-14 | 2 | 2 | 2 | 2 | 2 | 2 | 4 | 4 | 4 |
| Benzoyl | 0.1 | | | | | | | | |
| peroxide | | | | | | | | | |
| Tadalafil | | 0.1 | | | | | | | 0.4 |
| Retinyl | | | 0.1 | | | | | | |
| palmitate | | | | | | | | | |
| Bakuchiol | | | | 0.1 | | | | | |
| Curcumin | | | | | 0.1 | | | | |
| Neem oil | | | | | | 0.1 | 12 | | |
| Castor oil | | | | | | | | 12 | |
| Genamin ® | 2 | 2 | 2 | 2 | 2 | 2 | 4 | 4 | 4 |
| O 020 | | | | | | | | | |
| Special | | | | | | | | | |
| Cetiol ® B | 5.9 | 5.9 | 5.9 | 5.9 | 5.9 | 5.9 | | | |
| Benzoyl | | | | | | | | | 11.6 |
| benzoate | | | | | | | | | |
| Sodium | | | | | | | | | 1.3 |
| dioctyl | | | | | | | | | |
| sulfosuccinate | | | | | | | | | |
| Olivoil ® | | | | | | | | | 18.7 |
| glutamate | | | | | | | | | |
| Water | 90 | 90 | 90 | 90 | 90 | 90 | 80 | 80 | 60 |
| D_{N}50 [nm] | 74.1 | 77.8 | 39.2 | 35.0 | 74.7 | 69.5 | 86.5 | 107.0 | 64.4 |
| PDI | 0.157 | 0.163 | 0.171 | 0.138 | 0.163 | 0.160 | 0.178 | 0.287 | 0.223 |
| Zeta potential | +48.8 | +48.0 | +49.6 | +48.7 | +46.4 | +55.4 | +96.2 | +69.9 | -41.6 |
| [mV] | | | | | | | | | |

Representative results of particle size distribution in a sample of ND32, showing the percentage (per number) of core-shell nano-particles having hydrodynamic diameters in the range of 10-1,000 nm, are presented in FIG. 2B.

As can be seen, ND37 and ND38 do not contain a dedicated non-volatile liquid. It is believed that the WITC-miscible active agents neem oil and castor oil, respectively included in ND37 and ND38 in relatively high amounts of three-fold the WITC weight, additionally served to plasticize the pre-mix including the WITC.

### Example 9-11: Preparation of Core-Shell Nano-Particles Externally Shelled by Water-Soluble Active Agents

1 g collagen peptides powder and 2.1 g water were placed in a 20 ml glass vial and mixed at room temperature until complete dissolution.

In a separate 20 ml vial, 6.9 g of the core-shell nano-dispersion ND11 obtained in Example 6-II were placed, and the collagen solution providing the molecules of water-soluble active agents due to form the second shells was added. The contents of the vial were shaken by hand for 10 seconds until a homogeneous mixture was obtained. The composition of collagen-coated nano-elements so obtained (Collagen-ND11) is reported in Table 6-II, which also presents a composition of LMW HA-coated nano-elements (LMW HA-ND11), similarly prepared. A reference composition containing uncoated nano-elements (Uncoated-cND11) was also prepared, comprising 3.1 g water lacking any carrier-soluble active agent.

Three additional nano-dispersions were prepared, based on ND6 and ND14 (referred to as LMW HA-ND6 and LMW HA-ND14, respectively), to which LMW HA was added to achieve the concentrations reported in Table 6-II. Samples of LMW HA-ND6 and of LMW HA-ND14 were prepared, wherein the LMW HA solution was mixed with the ND6 or ND14 compositions by sonication. For comparison, another sample of LMW HA-ND6 with same concentrations of constituents was prepared by mixing the LMW HA solution with the ND6 composition by hand.

These compositions are also reported in Table 6-II, wherein the different components and amounts are specified. The values reported in the table correspond to the concentration of each component in wt. % by total weight of the nano-dispersion. D_{N}50 and zeta potential values of most compositions are also presented in Table 6-II.

TABLE 6-11

| | Composition | | | | |
|---|---|---|---|---|---|
| | | | LMW | LMW | LMW |
| | Uncoated- | Collagen- | HA- | HA- | HA- |
| Component | ND11 | ND11 | ND11 | ND6 | ND14 |
| PCL-14 | 6 | 6 | 6 | 8.65 | 4 |
| Oleyl amine | 5 | 5 | 5 | 8.65 | |
| Genamin ® O | | | | | 15.8 |
| 020 Special | | | | | |
| Citrofol ® All | 9 | 9 | 9 | 25.9 | |
| Glycerol | 14 | 14 | 14 | | |
| Propylene | 10 | 10 | 10 | 9.8 | |
| glycol | | | | | |
| HCl | 25 | 25 | 25 | | |
| Collagen | | 10 | | | |
| LMW HA | | | 1 | 2.9 | 1 |
| Water | 31 | 21 | 30 | 44.1 | 79.2 |
| D_{N}50 [nm] | 50.1 | 43.4 | 40.4 | N/A | 93.3 |
| Zeta potential | +58.6 | +43.6 | +54.2 | N/A | +34.9 |
| [mV] | | | | | |

As can be seen from the changes in zeta potential, the addition of active agents to previously prepared core-shell nano-particles decreased the charges perceived at the newly formed outer surface of the nano-particles, in support of modifications at this interface with the liquid carrier, hence indicating the formation of a 2^{nd} shell composed of the active agent. For instance, while nano-dispersion of uncoated nano-elements ND14 had a zeta potential of +56.0 mV (according to Table 4-II(A) of Example 6-II), it can be seen that coating these nano-elements with LMW HA decreased their zeta potential to +34.9 mV.

For reference, the zeta potential of 0.1 g of the collagen peptides dispersed in 9.9 g of distilled water was found to be −7.7 mV, indicating a satisfactory AZ between the zeta potential of the collagen intended to coat the nano-particles and of the nano-particles to be coated thereby, which allows the attachment of the collagen to the surface of the nano-particles previously including only a first shell. The zeta potential of a similarly prepared solution of LMW HA in water was measured and found to be −17 mV, resulting in an even higher AZ compared to the one observed for the preparation of the composition of collagen-coated nano-particles.

The speed at which the carrier-soluble active agent forming the second shell is mixed with the “uncoated” core-shell having only a first shell of positively chargeable amines is believed to contribute to the form of the core-multi-shells nano-particles that can be obtained.

FIG. 6A shows a CryoTEM analysis of the first sample of composition LMW HA-ND6, obtained by high shear mixing (i.e., sonication) of the LMW HA with the core-shell nano-elements of ND6. In the figure, the cores 610 comprising the PCL, seen as dark globules, bear a second shell 630, composed of LMW HA. As can be seen in FIG. 6A, the second shell can comprise a number of layers of the HA, formed one on top of the other.

FIG. 6B is another image captured by CryoTEM, of the second sample LMW HA-ND6 composition, wherein the LMW HA was mixed in manually with the core-shell nano-elements of ND6. In the image, two dark PCL cores 610 can be seen surrounded by a mutual shell 630′, also composed of layers of the LMW HA, forming a larger nano-element. It is believed that manual mixing might be too slow/low energy, and the shells of the active agent formed on the cores coalesce and merge into such larger particles. Hence, it is suggested that high energy mixing (e.g., high shear) is more favorable than low energy mixing methods for the formation of second shells surrounding individual cores, such core-multi-shells nano-elements therefore having a particle size distribution commensurate with the size of the individual core-shell, more easily remaining within the ranges of size suitable for transdermal delivery.

Similarly, the ND12 nano-particles, prepared in Example 6-II, were coated with various water-soluble active agents, and an uncoated ND12 composition was also prepared and used as reference. The ND12-based compositions, including their respective ingredients and concentrations thereof, are summarized in Table 7-1I, the concentration of each of the components being provided in wt. % by total weight of the nano-dispersion. D_{N}50 and zeta potential values of each composition are also presented in Table 7-11.

TABLE 7-II

| | Composition | | | | |
|---|---|---|---|---|---|
| | Uncoated- | LMW | Vitamin | Collagen- | Elastin- |
| | ND12 | HA-ND12 | C-ND12 | ND12 | ND12 |
| PCL-14 | 6 | 6 | 6 | 6 | 6 |
| Oleyl amine | 6 | 6 | 6 | 6 | 6 |
| Citrofol ® All | 18 | 18 | 18 | 18 | 18 |
| Glycerol | 14 | 14 | 14 | 14 | 14 |
| Propylene | 10 | 10 | 10 | 10 | 10 |
| glycol | | | | | |
| HCl | 25 | 25 | 25 | 25 | 25 |
| LMW HA | | 0.5 | | | |
| Vitamin C | | | 0.5 | | |
| Collagen | | | | 1 | |
| Elastin | | | | | 1 |
| Water | 21 | 20.5 | 20.5 | 20 | 20 |
| D_{N}50 [nm] | 72.2 | 67.5 | 66.1 | 72.8 | 61.6 |
| Zeta potential | +66.6 | +50.8 | +44.5 | +33.5 | +39.9 |
| [mV] | | | | | |

Nano-elements having a shell made of vitamins may serve for the preparation of dietary supplements, whereas outer shells containing collagen, elastin or HA, which are known as mucoadhesive proteins or polysaccharide, may serve to promote the retention of the nano-elements (and other active agents contained therein) at a desired site of delivery.

### Example 10-II: Conductivity Measurements

In order to further demonstrate the adsorption of the water-soluble active agents to the surface of the core-shell nano-particles, conductivities of samples of the various ND12-based compositions were measured using a conductivity meter and are presented in microSiemens(S) in Table 8-11.

The rationale for this study relies on the expectation that if two species are mixed, each independently having a conductivity as an electrolyte in a particular medium, the conductivity of the mixture is the sum of the relative contributions of the species, only if the species remain separate. In other words, if the conductivity of a mixture of species is not the sum of the individual conductivities of its constituents, it can be assumed that the species interact one with the other.

In the present case, it is assumed that the molecules of the water-soluble active agent are able to attach to the core-shell nano-particles, so that when core-shell nano-particles having a conductivity A at a given concentration in a medium are mixed with an active agent having a conductivity B at a given concentration in substantially the same medium, the conductivity of the mixture should be lower than the combined conductivities A+B.

Samples of the various water-soluble active agents were prepared in a carrier being a mixture of liquids substantially similar to the one in which the nano-particles of ND12 and their coated versions were prepared. The conductivities of the active agents alone, of the core-shell nano-particles prior to coating by the second shell of active agents, and of the active agent-coated nano-particles were measured and are all reported in Table 8-II.

TABLE 8-11

| | Conductivity [μS] | | |
|---|---|---|---|
| | Active agent | Uncoated | Active agent- |
| Sample | in carrier | nano- | coated nano- |
| contents | only | particles | particles |
| LMW HA-ND12 | 401 | 735 | 651 |
| Vitamin C-ND12 | 511 | 754 | 835 |
| Collagen-ND12 | 104 | 720 | 521 |
| Elastin-ND12 | 125 | 731 | 700 |

As can be seen from the above table, the conductivities of the uncoated nano-particles only containing the first shell formed by the hydrophilic portion of the SFAs, in the present case fatty amines, are substantially similar, as expected for similar samples of core-shell nano-particles in a similar carrier, without any specific externally applied active agents.

In contrast, the conductivities of each active agent alone, or of the core-shell nano-particles coated thereby, depended on the active agent (e.g., LMW HA, vitamin C, collagen and elastin) being considered. Noticeably, the conductivities of all the samples of the active agent-coated nano-particles exhibited conductivities that were lower than the sum of: the conductivity (A) of the core-shell nano-particles in the carrier and the conductivity (B) of the active agent in substantially the same carrier. These results support that the present method enables the preparation of core-shell nano-particles having a core made of a WITC material, a first shell made of a SFA and a second shell, interacting with the first shell, the second shell being made of a water-soluble active agent.

### III. Cellular Penetration of Nano-Elements

The cellular penetration of nano-elements of the present compositions was tested in vitro by incubating a composition including a fluorescent dye within its nano-elements in a cell culture, allowing monitoring by fluorescence microscopy. The penetration of the nano-elements was assessed in cortical neuronal cells isolated from newborn rodents.

### Materials

The materials used in the following study are listed in Table 9 below.

TABLE 9

| | | Catalogue/ |
|---|---|---|
| Commercial name/acronym | Supplier | CAS No. |
| B-27 ™ Supplement (50X), | Gibco ™, USA | 17504044 |
| serum free | | |
| GlutaMAX ™ | Gibco ™, USA | 35050038 |
| Neurobasal ™ Medium (NB) | Gibco ™, USA | 21103049 |
| TrypLE ™ Express Enzyme | Gibco ™, USA | 12604013 |
| Gentamicin sulfate | Alfa Aesar, USA | J62834.1 |
| HyClone ™ Hank's 1X Balanced | Cytiva, USA | SH30030.02 |
| Salt Solutions (HBSS) | | |
| Hyclone ™ Defined Fetal | Cytiva, USA | SH30070.03 |
| Bovine Serum (FBS) | | |
| (4-(2-hydroxyethyl)-1- | Biological | 7365-45-9 |
| piperazineethanesulfonic acid) | Industries, Israel | |
| (HEPES) | | |
| Poly-L-lysine (PLL) | Sigma Aldrich, | 25988-63-0 |
| | USA | |
| Dulbecco Phosphate-Buffered | Sartorius, | 02-023-1A |
| Saline (DPBS) | Germany | |

Study system

All solutions and equipment used in the cell culture study were sterile, all manipulations being performed in a laminar flow cabinet. All incubations were performed in a tissue culture incubator maintained at 37° C., with CO₂ at a concentration of 5% and 95% relative humidity. The following solutions were used during the study: Brain neuronal culture (BNC) medium containing Neurobasal™ Medium including 5 vol. % of FBS, 2 vol. % of B-27™ Supplement, 1 vol. % of GlutaMAX™ and 30 ppm of gentamicin sulfate; a supplemented NB (SNB) medium corresponding to the BNC medium excluding FBS; and a Dissociation solution containing 2 vol. % of HEPES in HBSS.

A 24-wells tissue culture plate was pre-treated as follows to receive the cells to be cultured therein. A round glass microscope cover slip was placed at the bottom of each well. 0.3 ml of a poly-L-lysine (PLL) solution at a concentration of 0.1 mg/mL was added to each well and the plate incubated at 37° C. for 3 hours while gently shaking to ensure full coating of the cover slips by the solution. The PLL was then aspirated, and the wells rinsed with NB medium to eliminate residual PLL. 0.5 ml of the BNC medium were then added to each rinsed well and the plate including the cover slips pre-treated to promote the adhesion of the cells to be cultivated therewith maintained in the incubator until used.

### Cell Culture Preparation

The cells were prepared according to the following procedure:

1. Tissue collection: two newborn C₅₇ black mice were terminated by cervical cuts, and their brains were rapidly dissected using disinfected standard scissors. The brain cuts were collected in a 35 mm petri dish filled with 5 ml of the Dissociation solution and kept on ice.

2. Dissociation: the contents of the petri dish were then transferred into a 15 ml centrifugation tube, the brain cuts were allowed to settle, followed by the removal and discarding of the supernatant. Dissociation solution was added to the isolated brain pieces to a total volume of 4 ml, to which were added 1 ml of TrypLE™ Express Enzyme so as to induce the separation of the neuronal cells from other tissues. The tube and its contents were maintained for 20 minutes at 37° C. in an incubator while being gently shaken. The supernatant was then carefully removed and replaced with 1.2 ml NB medium.

3. Trituration: the tissues were subsequently triturated to yield single cells by pipetting up and down through glass Pasteur pipettes having decreasing opening sizes to yield a homogeneous solution containing single cells. The obtained cell suspension was centrifuged for 5 minutes at 500 RPM, the supernatant was discarded, and the pellet resuspended in 2 ml of BNC medium. The cells resuspended for a rinsing cycle were again precipitated by centrifugation for 5 minutes at 500 RPM.

4. Resuspension: the supernatant was carefully removed, and the rinsed cells were re-suspended in 1.5 ml BNC medium and the cell concentration determined using a counting chamber and light stereo microscope at suitable magnification (Stemi 200-C by Olympus).

5. Culture: the suspended cells were added to wells of the pre-treated 24-well plate, including poly-L-lysine coated coverslips at the bottom of the wells, at a volume ensuring the seeding of about 80,000 cells per well. The plate comprising in each well 0.5 ml of the BNC medium and 80,000 neuronal cells was maintained in the incubator for 3 days. One day before the addition of the tested composition, the medium was aspired and replaced by 1 ml per well of SNB medium.

### Tested Composition

The composition used for the present assay was prepared as described in Example 2-I, with the addition of 2 mg of the fluorescent marker Nile red during the preparation of the WITC premix. The final composition contained: 4 wt. % of PCL-14, 1.3 wt. % of Labrafil® M 1944 CS, 2.7 wt. % of Tefose® 63, 12 wt. % of Cetiol® B, 0.004 wt. % of Nile red, 1.3 wt. % of sodium dioctyl sulfosuccinate, 18.7 wt. % of Olivoil® glutamate and 60 wt. % of water.

The nano-dispersion was then sterilized by passing through a syringe equipped with a 100 nm filter and was stored in closed containers at room temperature until used.

### In-Vitro Study

After 3 days of incubation of the neuronal cells in BNC medium and 1 day of incubation in SNB medium, the tested composition was added to the wells including the pre-treated cover slips to which the cells adhered during the incubation period. Samples were added to the wells in two repeats, 1 μl being added to yield a concentration of 0.1 vol. % and 10 μl being added to yield a concentration of 1 vol. %. The plate was then incubated at 37° C. for 20 minutes to allow penetration of the nano-elements into the neuronal cells.

At the end of the incubation with the tested composition, the cover slips were retrieved from their respective wells, rinsed with DPBS and placed in a 35 mm petri dish with 2 ml of DPBS, so that the cells could be studied under a microscope while still alive (e.g., within one hour of being transferred to the DPBS). The presence of the dye within the cells was determined by fluorescence microscopy (using BX43 Olympus microscope equipped with a fluorescence filter and measured at a wavelength of 594 nm using cellSens software).

### Results

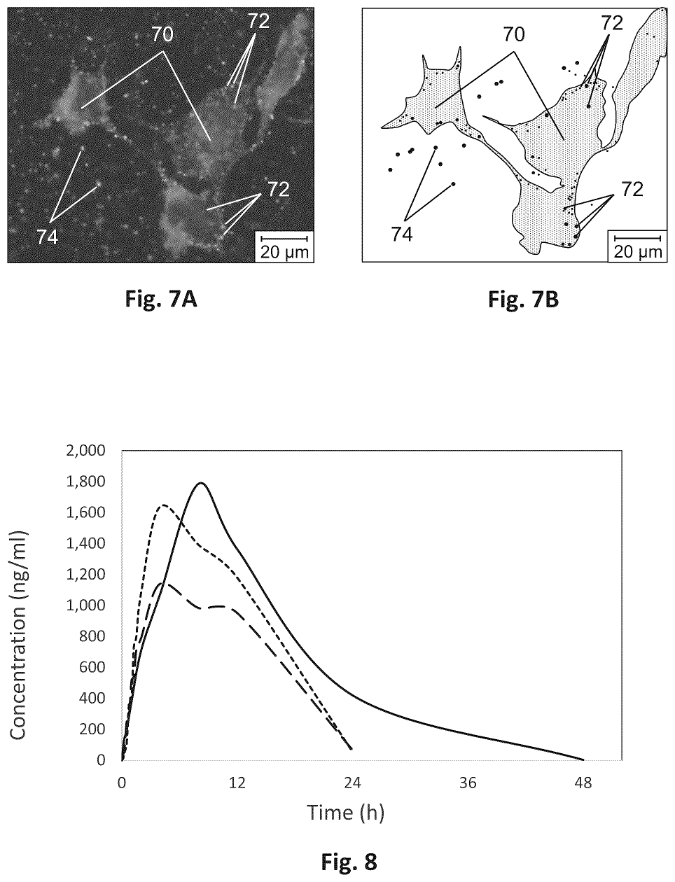

FIG. 7A shows cortical neuronal cells 70, and nano-elements containing the dye 72 can be seen within the neuronal cells, thus confirming the ability of the present nano-elements to penetrate into cells. Nano-elements 74 are also visible outside the cells, as can be expected considering that the composition containing them was applied externally onto the cell culture. FIG. 7B schematically depicts the cells of FIG. 7A, to better illustrate the cells, their outer membranes and the disposition of the nano-elements with respect to such biological barriers.

### IV. Use of the Compositions as Drug Nano-Carriers

The following pharmacokinetic (PK) study was conducted to determine the efficacy of a composition according to the present teachings, following its oral administration to rats.

### Study System

Nine young adult healthy rats (Sprague Dawley), having an initial mean body weight of about 243.1 +1.8 g, were used in this study. They were individually tail marked and housed within a limited access caged rodent facility. The rats were randomly divided into groups of 3, and were kept with their groups in cages, in a controlled environment at a temperature of 17-23° C., with a relative humidity (RH) of 30-70%, a 12:12 hour light:dark cycle and 15 air changes/h in the study room. They were provided free access to a commercial rodent diet and fresh drinking water during 5 acclimation days and throughout the entire study duration.

### Tested Composition and its Administration

Nano-dispersion ND39, as listed in Table 5-II, was used as the tested composition. ND39 contains tadalafil as the polar-carrier insoluble active agent present in the core of the nano-elements, and its concentration in the rats' blood was monitored.

1 ml of the composition, at a concentration of 4 mg/ml, yielding a dose of of about 16 mg/kg, was provided once, under anesthesia, to fed animals with a 2 ml syringe and gavage and blood samples were collected 0.25 hr, 0.5 hr, 1 hr, 2 hrs, 4 hrs, 8 hrs, 12 hrs, 24hrs and 48 hrs after administration of the nano-elements.

### Blood Collection

Three rats were bled at each time point via the retro-orbital sinus, and the blood was collected in EDTA Eppendorf tubes and immediately centrifuged for 5 minutes at 3,000 rpm. The plasma was collected by filtered pipette tips into vials and immediately stored at a temperature of −80° C.

### Termination

At the end of the study, the animals were euthanized by pentobarbitone sodium.

### PK Analysis

The plasma samples were combined with acetone, allowing the dissolution of the nano-elements and making the tadalafil available for measuring, followed by centrifugation whereby plasma proteins were precipitated. The obtained supernatant was removed and analyzed by LC-MS/MS vs. a tadalafil calibration curve prepared in advance, in the range of 1 ng/ml to 10 ug/ml.

The concentration of tadalafil (in ng/ml) as a function of time (in hours) following administration of the ND39 composition is presented as the black continuous line in the plot of FIG. 8. Also presented are pharmacokinetic data similarly obtained in a separate study after administering reference compositions to groups of 3 rats each. “Ref. C” relates to tablets of commercially available Cialis® containing tadalafil that were crushed and suspended in water at 2 mg/ml and administered at a dose of about 18.7 mg/kg (shown as a dashed line). “Ref. T” relates to an aqueous suspension of 2 mg/ml tadalafil bulk material, administered at a dose of about 18.1 mg/kg (shown as a dotted line).

As can be seen in FIG. 8, the levels of tadalafil delivered by the reference compositions decreased relatively rapidly, tadalafil being no longer detectible in the blood stream 24 hours following their administration. In contrast, the nano-elements of ND39 remained in the blood stream for a longer period of time, tadalafil extracted therefrom being no longer significantly detectible only 48 hours following their administration. This supports that an active agent carried by the present nano-elements may provide for a prolonged therapeutic activity of the drug, as it is released from the nano-elements, as compared to conventional treatments.

The area under the curves shown in the figure as assessed from zero to infinity (AUC_{0-∞}, in ng.hr/ml) and normalized to the doses of tadalafil of each group (_{0-∞}/D, in h.mg/mL) also suggest that the present nano-elements may be advantageous for the delivery of active agents. AUC_{0-∞}/D of the reference compositions were found to be approximately 930 h.mg/ml for Ref. C and about 1,249 h.mg/ml for Ref. T. In contrast, the AUC_{0-∞}/D calculated for the present composition was almost twice higher at about 1,897 h.mg/ml. This significant difference is attributed to the fact that in the present compositions, such as ND39 herein tested, the active agent, here tadalafil, is protected by the WITC of the core from which it is to be released, the tadalafil of Ref. C. and Ref. T lacking such protective nano-carriers.

Interestingly, the results obtained for the tested composition ND39 showed a smaller variation (as indicated by a calculated coefficient of variation (% CV) between the standard deviation and the mean values of all animals of each time point) which was found to be on average for all time points of only 17.7%. For comparison, the % CV values of Ref. C and Ref. T were found to be on average for all time points 57.5% and 31.1%, respectively, suggesting that the nano-elements of the composition can provide for a more repeatable effect.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the present disclosure has been described with respect to various specific embodiments presented thereof for the sake of illustration only, such specifically disclosed embodiments should not be considered limiting. Many other alternatives, modifications and variations of such embodiments will occur to those skilled in the art based upon Applicant's disclosure herein. Accordingly, it is intended to embrace all such alternatives, modifications and variations and to be bound only by the spirit and scope of the disclosure and any change which come within their meaning and range of equivalency.

In the description and claims of the present disclosure, each of the verbs “comprise”, “include” and “have”, and conjugates thereof, are used to indicate that the object or objects of the verb are not necessarily a complete listing of features, members, steps, components, elements or parts of the subject or subjects of the verb. Yet, it is contemplated that the compositions of the present teachings also consist essentially of, or consist of, the recited components, and that the methods of the present teachings also consist essentially of, or consist of, the recited process steps.

As used herein, the singular form “a”, “an” and “the” include plural references and mean “at least one” or “one or more” unless the context clearly dictates otherwise. At least one of A and B is intended to mean either A or B, and may mean, in some embodiments, A and B. A “material” that may be present in the composition alone or in combination with other materials of the same type can be referred to as “material(s)”; WITC(s), WITP(s), polar carrier(s), non-volatile liquid(s), surfactant(s), active agent(s) and the like, respectively indicating that at least one WITC, at least one WITP, at least one polar carrier, at least one non-volatile liquid, at least one surfactant, at least one active agent, and so on, can be used in the present methods or be included in the composition or satisfy the recited parameter or suitable range thereof.

Unless otherwise stated, the use of the expression “and/or” between the last two members of a list of options for selection indicates that a selection of one or more of the listed options is appropriate and may be made.

Unless otherwise stated, when the outer bounds of a range with respect to a feature of an embodiment of the present technology are noted in the disclosure, it should be understood that in the embodiment, the possible values of the feature may include the noted outer bounds as well as values in between the noted outer bounds.

As used herein, unless otherwise stated, adjectives such as “substantially”, “approximately” and “about” that modify a condition or relationship characteristic of a feature or features of an embodiment of the present technology, are to be understood to mean that the condition or characteristic is defined to within tolerances that are acceptable for operation of the embodiment for an application for which it is intended, or within variations expected from the measurement being performed and/or from the measuring instrument being used. When the term “about” and “approximately” precedes a numerical value, it is intended to indicate +/−15%, or +/−10%, or even only +/−5%, and in some instances the precise value. Furthermore, unless otherwise stated, the terms (e.g., numbers) used in this disclosure, even without such adjectives, should be construed as having tolerances which may depart from the precise meaning of the relevant term but would enable the invention or the relevant portion thereof to operate and function as described, and as understood by a person skilled in the art.

While this disclosure has been described in terms of certain embodiments and generally associated methods, alterations and permutations of the embodiments and methods will be apparent to those skilled in the art. The present disclosure is to be understood as not limited by the specific embodiments described herein.

Certain marks referenced herein may be common law or registered trademarks of third parties. Use of these marks is by way of example and shall not be construed as descriptive or limit the scope of this disclosure to material associated only with such marks.

1. A method of treating a living subject or an object, the method comprising administering via non-exposed surfaces of said subject or object a composition comprising nano-elements containing:
a) a core including at least one water-insoluble thermoplastic compound (WITC) and a non-volatile liquid miscible therewith; and
b) at least one active agent disposed at least in part in the core or in a shell surrounding the core, when a shell directly or indirectly surrounding the core is present;
wherein each constituent of the nano-elements has a vapor pressure of 40 Pascal (Pa) or less, as measured at a temperature of about 20° C.; and wherein the nano-elements, in absence or presence of a shell, i) are dispersible in a polar carrier; and ii) have an average diameter D_{N}50 of 1,000 nm or less.

2. The method as claimed in claim 1, wherein the nano-elements further contain at least one amphiphilic shell-forming agent (SFA), the, or each, SFA being miscible in the WITC(s) and insoluble in the polar carrier, a hydrophilic portion of the SFA(s) forming a first shell directly surrounding each core and the shell being chargeable.

3. The method as claimed in claim 2, wherein the SFA, or each SFA individually, is selected from a group consisting of fatty amines, fatty acids and metal salts of aryl alkyl sulfonates or petroleum sulfonates, and combinations thereof.

4. The method as claimed in claim 1, wherein at least one of the one or more active agents is miscible in the WITC(s) and insoluble in the polar carrier, said active agent(s) being contained in the core of the nano-elements, when apolar, or at least partially entrapped in the core of the nano-elements, when amphiphilic, a hydrophilic portion of amphiphilic active agents forming a first shell surrounding each core, in absence or presence of SFA(s).

5. The method as claimed in claim 2, wherein at least one of the one or more active agents is soluble in the polar carrier, said active agent(s) forming a second shell being anchored to the core of the nano-elements via the first shell.

6. The method as claimed in claim 1, wherein the WITC, or the blend of WITCs, and/or the nano-elements having a core made of the same, is/are characterized by at least one, at least two, or at least three of the following properties:
i. the WITC, or blend thereof, and/or the nano-elements is/are insoluble in the polar carrier;
ii. the WITC, or blend thereof, and/or the nano-elements is/are biodegradable and/or biocompatible;
iii. the WITC, or blend thereof, and/or the nano-elements has/have each respectively at least one of a first Tm and a second Tm between 0° C. and 300° C., the second Tm being lower than the first Tm;
iv. the WITC, or blend thereof, and/or nano-elements has/have each respectively at least one of a first Tg, a first Ts, a second Tg and a second Ts between −75° C. and 300° C., the second Tg or Ts being lower than the corresponding first Tg or Ts; and
v. the WITC, or each WITC individually, has a molecular weight between 0.6 kDa and 500 kDa.

7. The method as claimed in claim 1, wherein the WITC, or each WITC individually, is selected from:
(I) a polymer selected from a group of polymer families comprising aliphatic polyesters, polyhydroxy-alkanoates, poly(alkene dicarboxylates), polycarbonates, aliphatic-aromatic copolyesters, polysaccharides, lignins, isomers thereof, copolymers thereof and combinations thereof; and
(II) a natural polymerizable WITC selected from resins, gums, waxes and gum-resins.

8. The method as claimed in claim 1, wherein the active agent, or each active agent individually, has a molecular weight of up to 500 kDa.

9. The method as claimed in claim 1, wherein the composition is a medication containing an effective amount of the one or more active agents, the medication being:
I) for the treatment of a living subject, said treatment including diagnosing, preventing, ameliorating, attenuating, delaying or arresting a progression and/or curing an ailment in a subject in need thereof, the, or each, active agent being selected from the group comprising: analgesics, anesthetics, anti-addiction agents, anti-bacterials, anti-convulsants, anti-dementia agents, anti-depressants, anti-emetics, anti-fungals, anti-gout agents, anti-inflammatory agents, anti-migraine agents, anti-myasthenic agents, anti-mycobacterials, anti-neoplastics, anti-obesity agents, anti-parasitics, anti-Parkinson agents, anti-psychotics, anti-spasticity agents, anti-virals, anxiolytics, bipolar agents, blood glucose regulators, cardiovascular agents, central nervous system agents, contraceptives, dental and oral agents, gastrointestinal agents, genetic/enzyme/protein disorder agents, genitourinary agents, hormonal agents, hormone suppressant, immunological agents, infertility agents, inflammatory bowel disease agents, metabolic bone disease agents, ophthalmic agents, otic agents, respiratory tract agents, sexual disorder agents, skeletal muscle relaxants, sleep disorder agents and nutraceutical supplements; the medication optionally being in a pharmaceutical dosage form selected from the group comprising: solid dosage forms, semi-solid dosage-forms, and liquid dosage forms, said dosage forms being either ready for administration or for the preparation of final dosage forms at a time of administration; or
II) for the treatment of an object in need thereof, the, or each, active agent being selected from the group comprising: acaricides, algicides, anthelmintics, anti-moths, avicides, bactericides, chemo-sterilants, fertilizers, fungicides, herbicides, insecticides, insect repellents, insect pheromones, molluscicides, nematicides, nitrification inhibitors, ovicides, pesticides, pest repellents, plant growth promotors, rodenticides, termicides, virucides, and plant wound protectants; the medication optionally being in an agrochemical dosage form selected from the group comprising: solutions, dispersions, emulsions, or granules, pellets, or powders for preparing the same.

10. The method as claimed in claim 1, wherein the non-volatile liquid included in the core of the nano-elements is selected from a group comprising monofunctional or polyfunctional aliphatic esters, fatty esters, cyclic organic esters, terpenes, aromatic alcohols, aromatic esters, aromatic ethers, aldehydes, and combinations thereof.

11. The method as claimed in claim 1, wherein the nano-elements have a dynamic viscosity selected to release the active agent(s) from the nano-elements at a predetermined onset following the administration of the composition and/or for a desired duration of time; the dynamic viscosity of the nano-elements optionally being 10⁷ mPa·s or less, the nano-elements further optionally having a dynamic viscosity of 1 mPa·s or more, as measured at at least one temperature between 20° C. and 80° C., and at a shear rate of 10 sec⁻¹.

12. The method as claimed in claim 1, wherein each constituent of the nano-elements has a vapor pressure of 20 Pa or less, as measured at a temperature of about 20° C., the core of the nano-elements being non-porous.

13. The method as claimed in claim 1, wherein, in the presence of water and as measured at room temperature, the nano-elements have a positive or negative charge, having an absolute value of 5 mV or more; the absolute value of the charge of the nano-elements optionally being of 100 mV or less.

14. The method as claimed in claim 1, wherein the polar carrier in which the nano-elements are dispersible includes at least one polar liquid selected from a group consisting of water, glycols, glycerols, formamide, acetonitrile, and combinations thereof.

15. The method as claimed in claim 1, wherein the composition contains in addition to the nano-elements, a polar carrier in liquid form, the liquid optionally comprising at least one of i) a surfactant being an emulsifier or an hydrotrope; and ii) a pH modifying agent.

16. The method as claimed in claim 1, wherein the WITC, or each WITC, has a molecular weight between 0.6 kDa and 500 kDa and the average diameter D_{N}50 of the nano-elements is 200 nm or less; the nano-elements optionally having a D_{N}50 of at least 5 nm.

17. The method as claimed in claim 1, wherein the active agent, or each active agent, is a polar-carrier-insoluble and WITC-miscible active agent, disposed at least in part in the core of the nano-elements.

18. The method as claimed in claim 1, wherein the nano-elements are substantially devoid of a volatile organic compound (VOC), the nano-elements optionally containing less than 0.2 wt. % of a VOC, or blend thereof, by weight of the nano-elements.

19. The method as claimed in claim 1, wherein at least one of the one or more active agents is released from the nano-elements over an extended period of time of at least twelve hours, whereby the composition is a sustained-release medication with respect to said active agent(s).

20. The method as claimed in claim 1, wherein the nano-elements are prepared by a method comprising the steps of:
a) providing the at least one WITC, wherein:
i. the WITC, or a blend thereof, has at least one of a first melting temperature (Tm), a first softening temperature (Ts), and a first glass transition temperature (Tg) of 300° C. or less; and
ii. the WITC, or a blend thereof, has a first viscosity optionally higher than 10⁷ mPa·s, as measured at at least one temperature between 20° C. and 80° C., and at a shear rate of 10 sec−1;
b) mixing the at least one WITC with the non-volatile liquid and optionally SFA(s) when present, the mixing being at a mixing temperature equal to or higher than at least one of the first Tm, Ts, and Tg of the WITC(s), whereby a homogeneous mixture of a plasticized WITC(s), optionally including SFA(s) miscible therewith, is formed, the plasticized mixture having a second Tm, Ts, or Tg lower than the respective first Tm, Ts, or Tg, and a second viscosity lower than the first viscosity, at least one of the first and the second viscosity being of 10⁷ mPa·s or less, as measured at at least one temperature between 20° C. and 80° C., and a shear rate of 10 sec⁻¹;
c) combining a polar carrier with the plasticized mixture of step b) including at least the WITC(s); and
d) nano-sizing the combination of step c) by applying shear at a shearing temperature equal to or higher than at least one of the second Tm, Ts, and Tg of the plasticized WITC(s), so as to obtain a nano-suspension, whereby nano-elements including at least a core comprising the plasticized WITC(s), and optionally a first shell surrounding the core containing at least a hydrophilic portion of the SFA(s), are dispersed in the polar carrier;
wherein each active agent being miscible in the WITC(s) and insoluble in the polar carrier is combined with the WITC(s) during step b); and/or
wherein each active agent being soluble in the polar carrier is added during step c) or step d), provided that SFA(s) and/or amphiphilic active agent(s) were present in step b) to form a first shell, the polar-carrier soluble active agent(s) forming a second shell indirectly surrounding the core of the nano-elements and anchored thereto via the first shell.
